(19) United States
(12) Patent Application Publication
(10) Pub. No.: US 2025/0346638 A1
(43) Pub. Date: Nov. 13, 2025

(54) ENGINEERING IMMUNE RESPONSES

(71) Applicant: UNIVERSITY OF SOUTH CAROLINA, COLUMBIA, SC (US)

(72) Inventors: ASHLEY KING, COLUMBIA, SC (US); OLIVIA SHERRON, COLUMBIA, SC (US); HANNAH PONDER, COLUMBIA, SC (US); LYDIA PLESS, COLUMBIA, SC (US); VICTORIA BEATO, COLUMBIA, SC (US); NICHOLAS TRUEX, COLUMBIA, SC (US)

(21) Appl. No.: 19/175,665

(22) Filed: Apr. 10, 2025

Related U.S. Application Data
(60) Provisional application No. 63/632,110, filed on Apr. 10, 2024.

(51) Int. Cl. C07K 14/035; A61K 38/00; A61P 35/00

(52) U.S. Cl. CPC C07K 14/035; A61P 35/00; A61K 38/00; C07K 2319/09; C07K 2319/40; C07K 2319/81

(57) ABSTRACT

In general, disclosed herein are artificial transcription factors (ATFs) and methods thereof. Methods disclosed herein may include administering to a subject in need thereof an ATF disclosed herein. ATFs disclosed herein may include a DNA-binding domain and an activator domain. The DNA-binding domain may include from about 2 to about 8 zinc finger proteins. The zinc finger proteins may bind to a target site including SEQ ID NOs: 5-28, 30-34, 41-66, and/or 67-79.

### CROSS REFERENCE TO RELATED APPLICATIONS

This application claims the filing benefit of U.S. Provisional Patent Application Ser. No. 63/632,110, filed on Apr. 10, 2024, which is incorporated herein by reference.

### GOVERNMENT SUPPORT CLAUSE

This invention was made with government support under Grant No. R35 GM150565 awarded by National Institutes of Health (NIH). The government has certain rights in the invention.

### SEQUENCE LISTING

The instant application contains a Sequence Listing which has been submitted electronically in XML format and is hereby incorporated by reference in its entirety. Said XML copy, created on Apr. 23, 2025, is named USC-826_1699_SL.xml and is 110,366 bytes in size.

### BACKGROUND

Immune pathways are made up of intricate networks of peptides, proteins, and small molecules that regulate innate and adaptive immune responses but also prevent autoimmunity. Immunotherapies harnessing these pathways can generate powerful therapeutic responses but also suffer from serious off-target reactions that reduce efficacy and harm healthy tissue. Therefore, new strategies are desperately needed that boost immunotherapy responses, while reducing off-target activation, for these treatments to reach their full potential.

Cytokines and chemokines are important signaling proteins associated with immune cell development, activation, communication, differentiation, growth, and survival. These proteins are typically classified as either pro- or anti-inflammatory molecules, but can also serve polyfunctional roles depending on the magnitude and timing of expression. Tight regulation of cytokine genes is vital to limit chronic inflammation or unwanted immune tolerance mechanisms. Additionally, cytokine gene regulation is crucial for generating inflammation in response to infectious diseases and cancer. Immunotherapies often regulate cytokine expression by targeting cell surface receptors with adjuvants and immune-checkpoint blockade inhibitors, but reduced expression of the target receptors impairs immune function.

Strategies for immune activation typically include administering molecular adjuvants, or other inflammatory cues, composed of salt or oil emulsion mixtures, oligonucleotides, or lipids, which ultimately promote transcription and translation of inflammasome genes. These strategies are used widely across immunotherapy technologies, but often fail to generate optimal immune response landscapes and can even harm healthy tissue. Although genetic control is now possible with Clustered Regularly Interspaced Short Palindromic Repeats (CRISPR) technologies, therapeutic applications remain limited due to insufficient safety and intracellular delivery properties. New approaches to circumvent these challenges that provide direct immune regulation are needed to provide lower off-target effects through direct gene regulation without genome editing.

Transcription factors are essential signaling proteins that can regulate immunity and numerous other pathways, but currently, there are few strategies to regulate them individually. Transcription factors are critical in turning genes on or off, effectively controlling when, where, and how much of a gene's product is made. This regulatory function makes transcription factors essential for numerous biological processes, including development, cell differentiation, response to environmental signals, and maintenance of cellular identity. However, there is a lack of intracellular tools to directly activate genes instead of cell surface receptors. As such, there is a need in the art for artificial transcription factors (ATFs) to activate immune system genes directly. Disclosed herein are ATFs engineered to mimic or modulate the activity of natural transcription factors, the proteins that regulate gene expression by binding to specific DNA sequences.

### SUMMARY

In general, disclosed herein are artificial transcription factors (ATFs) and methods thereof. Methods disclosed herein may include administering to a subject in need thereof an ATF disclosed herein. ATFs disclosed herein may include a DNA-binding domain and an activator domain. The DNA-binding domain may include from about 3 to about 8 zinc finger proteins. The zinc finger proteins may bind to a target site including SEQ ID NOs: 5-28, 30-34, 41-66, and/or 67-70.

Also, disclosed herein are methods for activating the expression of an immune signaling gene in a cell. The method may include contacting the cell with an artificial transcription factor disclosed herein.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1A depicts the structure of a zinc finger domain.

FIG. 1B depicts representative structures of zinc finger domains and an ATF recognition of the DNA gene target (PDB ID: 2I13).

FIG. 1C depicts a scheme of an artificial transcription factor (ATF) protein.

FIG. 1D illustrates the promotor region of the hIFNG gene (NCBI Gene ID: 3,458, GRCh38.p14, human chromosome 12), depicting the target base pairs (bp) of the IFN-γ promoter region relative to the start codon.

FIG. 1E illustrates ATF-mediated transcriptional activation and protein expression.

FIG. 1F depicts gene-encoded ATF designs for mammalian expression.

FIG. 1G depicts gene-encoded ATF designs for bacterial expression.

FIG. 1H depicts TNF-encoded ATF designs for mammalian expression.

FIG. 1I illustrates interaction between the promoter region of the human TNF gene and ATF protein, including TNF start codon and first three amino acids.

FIG. 1J illustrates ATF-mediated transcriptional activation and protein expression.

FIG. 2 depicts a fluorescence microscopy panel after ATFs 1-4 transfections with Jurkat cells. Average transfection efficiencies are reported for each treatment (n=3); scale bars=250 μm.

FIG. 3A depicts ATF activation of IFN-γ expression in human Jurkat T cells. IFN-γ concentrations were determined by ELISA after transfecting Jurkat cells with buffer (vehicle), plasmid-encoded DsRed, or plasmid-encoded ATFs 1-4, followed by incubation with phorbol 12-Myristate 13-Acetate (PMA)/ionomycin, 3 μg/mL of brefeldin A (BFA), and 10 μM ZnCl₂. Plot shows IFN-γ concentrations after 24 hours. Statistical significance was evaluated by ANOVA analysis with a Holm-Sidak test to compare each ATF treatment versus the ‘vehicle’ treatment (*p<0.05; **p<0.005; ***p<0.001; and ****p<0.0001).

FIG. 3B depicts ATF activation of IFN-γ expression in human Jurkat T cells. IFN-γ concentrations were determined by ELISA after transfecting Jurkat cells with buffer (vehicle), plasmid-encoded DsRed, or plasmid-encoded ATFs 1-4, followed by incubation with phorbol 12-Myristate 13-Acetate (PMA)/ionomycin, 3 μg/mL of brefeldin A (BFA), and 10 μM ZnCl₂. Plot shows time-course monitoring of IFN-γ concentration at 12, 24, and 48 hours. Data represent the mean value of three replicate wells ±standard deviation (SD). Statistical significance was evaluated by ANOVA analysis with a Holm-Sidak test to compare each ATF treatment versus the ‘vehicle’ treatment (*p<0.05; **p<0.005; ***p<0.001; and ****p<0.0001).

FIG. 4A depicts scatterplot of t-statistics comparing the differences in gene transcription levels between IFNG targeting ATF 3 versus (vs.) vehicle (X-axis) and ATF 3 vs. Aart₆ (Y-axis). Artificial transcription factors activate IFNG and interferon-stimulated genes. Labels indicate IFNG and upregulated genes associated with human leukocyte antigens (HLAs) and interferon-stimulated genes (ISGs).

FIG. 4B depicts heatmap displaying the log-transformed values from the comparison across three samples, highlighting differentially expressed genes.

FIG. 4C depicts MA plot showing differentially expressed genes (DEGs) from ATF 3 compared to Aart₆.

FIG. 5A depicts native PAGE analysis of mixtures containing ATF 3r and synthetic double-stranded DNA (dsDNA) encoding IFNG (top) or an irrelevant sequence (bottom).

FIG. 5B depicts a plot showing the percentages of protein concentration (nM) versus free DNA or duplexed ATF-DNA. Data from ATF 3r forming duplexed ATF-DNA was fitted to a 1:1 binding model, which showed an apparent K_{D} of 5.2±0.3 nM. K_{D} was determined by a nonlinear fit of specific binding with Hill slope.

FIG. 6A depicts circular dichroism (CD) spectra from 190 to 250 nm show apo-ATF 3r and holo-ATF 3r.

FIG. 6B depicts thermal stability of ATF 3r. Differential scanning fluorimetry (DSF) of apo- and holo-ATF 3r. First derivative of relative fluorescence, indicating the protein melting point (Tₘ=52° C.).

FIG. 7A depicts DNA promoter region of the hIFNG gene. Gene sequence shows the IFN-γ promoter region from −426 to +15 base pairs, which was obtained from the National Center for Biotechnology Information (NCBI) from gene ID: 3458, assembly: GRCh38.p14, annotation release: RS_2023_10.

FIG. 7B depicts DNA promoter region of the hTNF gene. Gene sequence shows the TNF promoter region from −1142 to +15 base pairs, obtained from the National Center for Biotechnology Information (NCBI) from gene ID: 7,124, assembly: GRCh38.p14, annotation release: RS_2024_08.

FIG. 8 depicts quantification of PMA/ionomycin (stim) induced activation of IFN-γ by enzyme-linked immunosorbent assay (ELISA), with (+) and without (−) brefeldin A (BFA) treatment.

FIG. 9 depicts ELISA measuring IFN-γ from immortalized human Jurkat T cells in the presence of zinc ions (Zn).

FIG. 10 depicts purification of SUMO-ATF 3r from bacterial expression (E. coli).

FIG. 11 depicts cleavage of SUMO-ATF 3r by ulp-1 at a ratio of 100:1.

FIG. 12 depicts purification of ATF 3r.

FIG. 13 depicts LC-MS characterization of ATF 3r.

FIG. 14 depicts ELISA measuring TNF concentration after 18 hours and 24 hours after transfecting Jurkat T cells with buffer (‘shock’, vehicle) or plasmid-encoded ATFs 1-4, followed by incubation with Cell Stimulation Cocktail (‘stim’) or CD3/CD28 Dynabeads. Samples were supernatant following well collection and centrifugation.

FIG. 15 depicts TNF concentrations determined by ELISA after transfecting Jurkat T cells with buffer (‘shock’, vehicle) or TNF ATFs 1-4. Cells were treated with Cell Stimulation Cocktail and Brefeldin A one hour post transfection. Samples were lysate following well collection and cell lysis.

Repeat use of reference characters in the present specification and drawings is intended to represent the same or analogous features or elements of the present invention.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment may be used in another embodiment to yield a still further embodiment. Thus, it is intended that the present disclosure covers such modifications and variations as come within the scope of the appended claims and their equivalents.

In general, disclosed herein are methods for treating a disease characterized by an immune dysfunction. Methods disclosed herein may include administering to a subject in need thereof an artificial transcription factors (ATFs). Beneficially, ATFs disclosed herein may be useful in regulating immune pathways. For instance, ATFs disclosed herein may enable direct control of gene transcription and, in turn, translation of key proteins associated with immune cell signaling, differentiation, and migration pathways. Additionally, ATFs disclosed herein may be useful for upregulating immunogenic antigens and enabling chemical control of the timing and magnitude of their function.

In some example embodiments, ATFs disclosed herein may include a DNA-binding domain and an activator domain. For instance, in one example embodiment, the activator domain may be directly conjugated to the C-terminus of the DNA-binding domain without any intervening amino acid sequences. Alternatively, in another example embodiment, the DNA-binding domain may be directly conjugated to the N-terminus of the activator domain without any intervening amino acid sequences.

In another example embodiment, the DNA-binding domain and the activator domain may be conjugated using a peptide linker. For instance, the DNA-binding domain may be conjugated to the activator domain via a peptide linker having from about one (1) to one hundred (100) amino acids, such as from about five (5) to about ninety (90) amino acids, such as from about ten (10) to about seventy-five (75) amino acids, such as from about twenty (20) to about fifty (50) amino acids, or any range therebetween. In one example embodiment, the DNA-binding domain may be conjugated to the activator domain via a peptide linker having from about 1 to 100 amino acids. In another example embodiment, the DNA-binding domain may be conjugated to the activator domain via a peptide linker having from about 5 to 90 amino acids. In yet another example embodiment, the DNA-binding domain may be conjugated to the activator domain via a peptide linker having from about 10 to 75 amino acids. In another example embodiment, the DNA-binding domain may be conjugated to the activator domain via a peptide linker having from about 20 to 50 amino acids.

In one example embodiment, the DNA-binding domain and the activator domain may be conjugated with naturally occurring intervening residues found in the native proteins from which the domains are derived. In another example embodiment, the DNA-binding domain and the activator domain may be conjugated via a synthetic or exogenous linker sequence. For instance, the linker may be flexible, cleavable, non-cleavable, hydrophilic, and/or hydrophobic. In another example embodiment, the DNA-binding domain and the activator domain may be fused together via a linker comprising a plurality of glycine and/or serine residues.

### DNA-Binding Domain

The artificial transcription factor disclosed herein may include a DNA-binding domain. The DNA-binding domain may recognize and/or bind to a particular gene of interest. In one example embodiment, the DNA-binding domain may recognize and/or bind to a site of interest capable of modulating expression from a gene of interest when bound to an artificial transcription factor disclosed herein. For instance, the DNA-binding domain may recognize and/or bind to a site of interest capable of upregulating or downregulating expression of a particular gene of interest. In another example embodiment, the DNA-binding domain may recognize a genomic location and modulate expression of an endogenous gene when bound to an artificial transcription factor disclosed herein. Binding sites capable of modulating expression of an endogenous gene of interest when bound by an artificial transcription factor disclosed herein may be located anywhere in the genome that results in modulation of gene expression of the target gene. For instance, in some example embodiments, the binding site may be located on a different chromosome from the gene of interest or on the same chromosome as the gene of interest. In another example embodiment, the binding site may be located upstream of the transcriptional start site (TSS) of the gene of interest or downstream of the TSS of the gene of interest. In yet another example embodiment, the binding site may be located proximal to the TSS of the gene of interest or distal to the gene of interest. In yet another example embodiment, the binding site may be located within the coding region of the gene of interest or within an intron of the gene of interest. In another example embodiment, the binding site may be located downstream of the polyA tail of a gene of interest. In yet another example embodiment, the binding site may be located within a promoter sequence that regulates the gene of interest, within an enhancer sequence that regulates the gene of interest, or within a repressor sequence that regulates the gene of interest. In one example embodiment, DNA-binding domain may activate a gene of interest by recognizing and/or binding to the promoter region of said gene.

The DNA-binding domain may include, but is not limited to, a zinc finger protein, transcription activator-like effectors (TALEs), a CRISPR/Cas DNA binding complex, or a combination thereof. In one example embodiment, the DNA-binding domain may include a zinc finger protein. In another example embodiment, the DNA-binding domain may include a TALE. In yet another example embodiment, the DNA-binding domain may include a CRISPR/Cas DNA binding complex.

In one example embodiment, the DNA binding domain may include one or more zinc finger proteins. As used herein, a “zinc finger protein” refers to a protein, or a domain within a larger protein, that binds DNA in a sequence-specific manner through one or more zinc fingers, which are regions of amino acid sequence within the binding domain whose structure is stabilized through coordination of a zinc ion (Zn²⁺). The zinc finger motif allows for various combinations of DNA sequences to be bound with high degree of affinity and specificity, and is therefore ideally suited for engineering protein that can be targeted to and bind specific DNA sequences. The DNA-binding domain may include multiple zinc finger motifs, each recognizing a distinct triplet sequence, which collectively define a longer target sequence.

The zinc finger protein may be a naturally occurring or non-naturally occurring zinc finger protein. For instance, in one example embodiment, the zinc finger protein may be a naturally occurring zinc finger protein. In another example embodiment, the zinc finger protein may be a non-naturally occurring zinc finger protein that it is engineered to bind to a target site of choice.

In some example embodiments, the DNA binding domain may include from about two (2) to about eight (8) zinc finger proteins each recognizing a distinct triplet sequence, which collectively define a longer target sequence, such as from about three (3) to about eight (8) zinc finger proteins, such as from about four (4) to about six (6) zinc finger proteins, or any range therebetween. In one example embodiment, the DNA binding domain may have two (2) to about eight (8) zinc finger proteins. In another example embodiment, the DNA binding domain may have from about four (4) to about six (6) zinc finger proteins. For instance, in one example embodiment, the DNA binding domain may have at least two (2) zinc finger proteins. In another example embodiment, the DNA binding domain may have at least three (3) zinc finger proteins. In another example embodiment, the DNA binding domain may have at least four (4) zinc finger proteins. In another example embodiment, the DNA binding domain may have at least five (5) zinc finger proteins. In another example embodiment, the DNA binding domain may have at least six (6) zinc finger proteins. In another example embodiment, the DNA binding domain may have at least seven (7) zinc finger proteins. In another example embodiment, the DNA binding domain may have at least eight (8) zinc finger proteins.

In some example embodiments, each zinc finger protein in the DNA binding domain may be linked to another zinc finger protein or another domain at either its N-terminus or C-terminus. In another example embodiment, each zinc finger protein in the DNA binding domain may be linked to another zinc finger protein or another domain via an amino acid linker. In some example embodiments, zinc finger proteins disclosed herein may have a sequence disclosed in Tables 1-6 herein.

Zinc finger proteins recognize specific nucleotide triplets (e.g., 3 base pairs (bp)). As such, the number of zinc fingers proteins in the DNA binding domain may inform the length of the binding site recognized by the DNA binding domain. In some example embodiments, the DNA binding domain may recognize a target binding site having from about 9 bp to about 24 bp. For instance, in one example embodiment, a DNA binding domain with 3 zinc finger proteins may bind to a genomic region site having 9 bp. In another example embodiment, a DNA binding domain with 4 zinc fingers may bind to a genomic region site having 12 bp. In yet another example embodiment, a DNA binding domain with 5 zinc fingers may bind to a genomic region site having 15 bp. In another example embodiment, a DNA binding domain with 6 zinc fingers may bind to a genomic region site having 18 bp. In another example embodiment, a DNA binding domain with 7 zinc fingers may bind to a genomic region site having 21 bp. In another example embodiment, a DNA binding domain with 8 zinc fingers may bind to a genomic region site having 24 bp. In general, a DNA binding domain that recognizes a longer target binding site will exhibit greater binding specificity (e.g., less off target or non-specific binding).

In one example embodiment, the zinc finger proteins may bind to a target site corresponding to an amino acid sequence set forth in SEQ ID NOs: 5-28 and/or 30-34, or a functionally equivalent variant thereof. For instance, in one example embodiment, the zinc finger proteins may bind to one or more target sites corresponding to an amino acid sequence set forth in SEQ ID NOs: 5-28 and/or 30-34, or a functionally equivalent variant thereof. For instance, the functionally equivalent variant may be at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to an amino acid sequence set forth in SEQ ID NOs: 5-28 and/or 30-34. In one example embodiment, the functionally equivalent variant may be at least 70% identical to an amino acid sequence set forth in SEQ ID NOs: 5-28 and/or 30-34. In another example embodiment, the functionally equivalent variant may be at least 80% identical to an amino acid sequence set forth in SEQ ID NOs: 5-28 and/or 30-34. In another example embodiment, the functionally equivalent variant may be at least 85% identical to an amino acid sequence set forth in SEQ ID NOs: 5-28 and/or 30-34. In another example embodiment, the functionally equivalent variant may be at least 90% identical to an amino acid sequence set forth in SEQ ID NOs: 5-28 and/or 30-34. In another example embodiment, the functionally equivalent variant may be at least 95% identical to an amino acid sequence set forth in SEQ ID NOs: 5-28 and/or 30-34. In another example embodiment, the functionally equivalent variant may be at least 98% identical to an amino acid sequence set forth in SEQ ID NOs: 5-28 and/or 30-34.

In one example embodiment, the zinc finger proteins may bind to a target site corresponding to a nucleotide sequence set forth in SEQ ID NOs: 71-75, or a functionally equivalent variant thereof. For instance, in one example embodiment, the zinc finger proteins may bind to one or more target sites corresponding to a nucleotide sequence set forth in SEQ ID NOs: 71-75, or a functionally equivalent variant thereof. For instance, the functionally equivalent variant may be at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a nucleotide sequence set forth in SEQ ID NOs: 71-75. In one example embodiment, the functionally equivalent variant may be at least 70% identical to a nucleotide sequence set forth in SEQ ID NOs: 71-75. In another example embodiment, the functionally equivalent variant may be at least 80% identical to a nucleotide sequence set forth in SEQ ID NOs: 71-75. In another example embodiment, the functionally equivalent variant may be at least 85% identical to a nucleotide sequence set forth in SEQ ID NOs: 71-75. In another example embodiment, the functionally equivalent variant may be at least 90% identical to a nucleotide sequence set forth in SEQ ID NOs: 71-75. In another example embodiment, the functionally equivalent variant may be at least 95% identical to a nucleotide sequence set forth in SEQ ID NOs: 71-75. In another example embodiment, the functionally equivalent variant may be at least 98% identical to a nucleotide sequence set forth in SEQ ID NOs: 71-75.

In one example embodiment, the zinc finger proteins may bind to a target site corresponding to an amino acid sequence set forth in SEQ ID NOs: 43-66 and/or 67-70, or a functionally equivalent variant thereof. For instance, in one example embodiment, the zinc finger proteins may bind to one or more target sites corresponding to an amino acid sequence set forth in SEQ ID NOs: 43-66 and/or 67-70, or a functionally equivalent variant thereof. For instance, the functionally equivalent variant may be at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to an amino acid sequence set forth in SEQ ID NOs: 43-66 and/or 67-70. In one example embodiment, the functionally equivalent variant may be at least 70% identical to an amino acid sequence set forth in SEQ ID NOs: 43-66 and/or 67-70. In another example embodiment, the functionally equivalent variant may be at least 80% identical to an amino acid sequence set forth in SEQ ID NOs: 43-66 and/or 67-70. In another example embodiment, the functionally equivalent variant may be at least 85% identical to an amino acid sequence set forth in SEQ ID NOs: 43-66 and/or 67-70. In another example embodiment, the functionally equivalent variant may be at least 90% identical to an amino acid sequence set forth in SEQ ID NOs: 43-66 and/or 67-70. In another example embodiment, the functionally equivalent variant may be at least 95% identical to an amino acid sequence set forth in SEQ ID NOs: 43-66 and/or 67-70. In another example embodiment, the functionally equivalent variant may be at least 98% identical to an amino acid sequence set forth in SEQ ID NOs: 43-66 and/or 67-70.

In one example embodiment, the zinc finger proteins may bind to a target site corresponding to a nucleotide sequence set forth in SEQ ID NOs: 76-79, or a functionally equivalent variant thereof. For instance, in one example embodiment, the zinc finger proteins may bind to one or more target sites corresponding to a nucleotide sequence set forth in SEQ ID NOs: 76-79, or a functionally equivalent variant thereof. For instance, the functionally equivalent variant may be at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a nucleotide sequence set forth in SEQ ID NOs: 76-79. In one example embodiment, the functionally equivalent variant may be at least 70% identical to a nucleotide sequence set forth in SEQ ID NOs: 76-79. In another example embodiment, the functionally equivalent variant may be at least 80% identical to a nucleotide sequence set forth in SEQ ID NOs: 76-79. In another example embodiment, the functionally equivalent variant may be at least 85% identical to a nucleotide sequence set forth in SEQ ID NOs: 76-79. In another example embodiment, the functionally equivalent variant may be at least 90% identical to a nucleotide sequence set forth in SEQ ID NOs: 76-79. In another example embodiment, the functionally equivalent variant may be at least 95% identical to a nucleotide sequence set forth in SEQ ID NOs: 76-79. In another example embodiment, the functionally equivalent variant may be at least 98% identical to a nucleotide sequence set forth in SEQ ID NOs: 76-79.

In one example embodiment, the DNA binding domain may include a TALE nuclease. As used herein, a “TALE nuclease” refers to a bacterial protein derived from Xanthomonas bacteria that have a DNA binding domain and a nuclease domain that can be engineered to cut specific sequences of DNA. TALE nucleases function as sequence-specific transcription factors by directly recognizing DNA bases. TALE nucleases may be engineered to bind to a desired target DNA sequence thereby directing the nuclease domain to a specific location. In general, TALE nucleases have 34 amino acids, with variation occurring primarily at two key residues, the 12th and 13th amino acids. These two key residues are known as repeat-variable diresidues (RVDs) that determine specificity for a particular DNA base.

In one example embodiment, the DNA binding domain may include a CRISPR/Cas DNA binding complex. For instance, the DNA binding domain may be include a guide RNA and a nuclease inactivated Cas protein. In one example embodiment, the nuclease inactivated Cas protein may be a nuclease inactivated Cas9.

### Activator Domain

In some example embodiments, the artificial transcription factor disclosed herein may include an activator domain. As used herein, an “activator domain” refers to a domain of a protein which in conjunction with a DNA binding domain can activate transcription from a promoter by contacting transcriptional machinery (e.g., general transcription factors and/or RNA polymerase) either directly or through other proteins known as co-activators. In one example embodiment, the activator domain may be a synthetically designed domain. In another example embodiment, the activator domain may be derived from a naturally occurring protein, e.g., a transcription factor, a transcriptional co-activator, a transcriptional co-repressor, or a silencer protein. For instance, the activator domain may be derived from a protein of any species, e.g., a mouse, rat, monkey, virus, or human.

In one example embodiment, the activator domain may be derived from one or more viral proteins. For instance, the one or more viral protein may include, but is not limited to, Herpes Simplex Viral Protein 16 (VP16), Herpes Simplex Viral Protein 64 (VP64), VP128, p65, p300, or a combination thereof. In one example embodiment, the activator domain may be derived from VP64. As used herein, “VP64” refers to a transcriptional activator composed of four tandem copies of VP16, generally connected with glycine-serine linkers. When fused to another protein domain that can bind near the promoter of a gene, VP64 acts as a strong transcriptional activator.

In one example embodiment, the activator domain may include one or more nucleic acid sequences encoding one or more nuclear localization signals (NLS). Any NLS peptide that facilitates import of the protein to which is attached into the cell nucleus may be used. For instance, the NLS sequence may be derived from various transcription factors and viral proteins known to enhance nuclear retention and gene regulation, including, but are not limited to, Simian Virus 40 (SV40) Large T-Antigen, the nucleoplasmin NLS, EGL-13 NLS, c-Myc NLS, TUS-protein NLS, NF-κB p65 NLS, or a combination thereof. In one example embodiment, the NLS sequence may be derived from SV40. In another example embodiment, the NLS sequence may be derived from the nucleoplasmin NLS. In yet another example embodiment, the NLS sequence may be derived from EGL-13 NLS. In another example embodiment, the NLS sequence may be derived from c-Myc NLS. In another example embodiment, the NLS sequence may be derived from TUS-protein NLS. In another example embodiment, the NLS sequence may be derived from NF-κB p65 NLS.

In one example embodiment, the activator domain may include a protein tag. For instance, the protein tag may be an epitope tag. In one example embodiment, the epitope tag may include, but is not limited to, a hemagglutinin A (HA) tag, FLAG, or a combination thereof. For instance, in one example embodiment, the protein tag may be an HA tag. In another example embodiment, the protein tag may be FLAG.

In one example embodiment, the artificial transcription factor may encode an activator domain with three signals: a nuclear localization signal (NLS), four tandem copies of virus protein 16 (VP64), and a hemagglutinin A (HA) tag.

### Fluorescent Domain/Mammalian Expression

Optionally, in some example embodiments, the artificial transcription factor disclosed herein may include a fluorescent domain. As used herein, a “fluorescent domain” refers to an amino acid sequence, or a nucleotide sequence that encodes the amino acid sequence, which fluoresces and/or emits a certain wavelength of light when exposed to an excitation wavelength. In one example embodiment, the fluorescent domain may be directly conjugated to the N-terminus of the DNA-binding domain. Alternatively, in another example embodiment, the DNA-binding domain may be directly conjugated to the C-terminus of the fluorescent domain.

In general, the fluorescent domain may enable real-time visualization, tracking, monitoring intracellular localization, and quantification of proteins in various applications including, but are not limited to, living cells, biochemical assays, and imaging applications. In one example embodiment, the fluorescent domain of the artificial transcription factor disclosed herein may encode a fluorescent protein, a luciferase, a β-galactosidase, a chloramphenicol acetyltransferase (CAT), a β-glucuronidase (GUS), or a combination thereof. For instance, in one example embodiment, the fluorescent domain of the artificial transcription factor disclosed herein may encode a fluorescent protein. In another example embodiment, the fluorescent domain of the artificial transcription factor disclosed herein may encode a luciferase. In another example embodiment, the fluorescent domain of the artificial transcription factor disclosed herein may encode a β-galactosidase.

In one example embodiment, the fluorescent protein may include, but is not limited to, a green fluorescent protein (GFP), an enhanced green fluorescent protein (EGFP), a super-fold GFP (sfGFP), a red fluorescent protein (RFP), a cyan fluorescent protein (CFP), a blue green fluorescent protein (BFP), an enhanced blue fluorescent protein (EBFP), a yellow fluorescent protein (YFP), or a combination thereof. For instance, the fluorescent protein may include, but is not limited to, AcGFP, TurboGFP, Emerald, Azami Green, ZsGreen, EBFP, Sapphire, T-Sapphire, ECFP, mCFP, Cerulean, CyPet, AmCyan1, Midori-Ishi Cyan, mTFPT (Teal), enhanced yellow fluorescent protein (EYFP), Topaz, Venus, mCitrine, YPet, PhiYFP, ZsYellow1, mBanana, Kusabira Orange, mOrange, dTomato, dTomato-Tandem, Discosoma red (DsRed), DsRed2, DsRed-Express (T1), DsRed-Monomer, mTangerine, mStrawberry, AsRed2, mRFP1, JRed, mCherry, HcRed1, mRaspberry, HcRed1, HcRed-Tandem, mPlum, AQ143, or a combination thereof. In one example embodiment, the fluorescent protein may be DsRed. In another example embodiment, the fluorescent protein may be DsRed2. In yet another example embodiment, the fluorescent protein may be dTomato. In another example embodiment, the fluorescent protein may be AcGFP.

Optionally, the fluorescent domain of the artificial transcription factor disclosed herein may include a fluorescent domain and a ribosome skip element. For instance, the ribosome skip element may be spaced in between the fluorescent domain and the DNA binding domain of the artificial transcription factor. In one example embodiment, the ribosome skip element may be a nucleic acid sequence encoding a ribosome skip element. For instance, in one example embodiment, the nucleic acid sequence encoding a ribosome skip element may be a 2A peptide including but is not limited to, a Thosea asigna 2A peptide (T2A), a Porcine teschovirus-1 2A peptide (P2A), an Equine rhinitis A virus 2A peptide (E2A), a Foot-and-mouth disease virus 2A peptide (F2A), or a combination thereof. In one example embodiment, the 2A peptide may be T2A. In another example embodiment, the 2A peptide may be P2A. In yet another example embodiment, the 2A peptide may be E2A. In another example embodiment, the 2A peptide may be F2A. The ribosome skipping element, such as a T2A, may cause the ribosome to skip (ribosome skipping) synthesis of a peptide bond at the C-terminus of a 2A element, leading to separation between the end of the 2A sequence and the next peptide downstream. As such, the ribosome skipping element allows the inserted transgene to be controlled by the transcription of the endogenous promoter at the integration site.

### Purification Domain/Mammalian Expression

Artificial transcription factors disclosed herein may be produced using various standard recombinant techniques. For instance, such techniques use vectors, such as expression vectors, that includes a nucleic acid encoding an artificial transcription factor disclosed herein. As used herein, a “vector” refers to a nucleic acid molecule capable of transporting another nucleic acid to which it has been linked to a cell. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). Also, other forms of expression vectors, such as viral vectors (e.g., replication defective retroviruses, adenoviruses, and adeno-associated viruses), which serve equivalent functions.

In one example embodiment, an artificial transcription factor disclosed herein may be expressed via recombinant expression vectors suitable for expression of the artificial transcription factor in a host cell. For instance, the recombinant expression vectors may include one or more regulatory sequences, selected on the basis of the host cells to be used for expression, which is operably linked to the nucleic acid sequence which encodes an artificial transcription factor disclosed herein to be expressed. Within a recombinant expression vector, an “operably linked” refers to the nucleotide sequence of interest being linked to the regulatory sequence(s) in a manner which allows for expression of the nucleotide sequence (e.g., in an in vitro transcription/translation system or in a host cell when the vector is introduced into the host cell). It will be appreciated by those skilled in the art that the design of the expression vector can depend on such factors as the choice of the host cell to be transformed, the level of expression of protein desired, and the like. The expression vectors of the present invention can be introduced into host cells to thereby produce artificial transcription factors. proteins or peptides, including fusion proteins or peptides, encoded by nucleic acids as described herein.

The recombinant expression vectors may be designed for expression of a polypeptide corresponding to an artificial transcription factor disclosed herein in prokaryotic (e.g., E. coli) or eukaryotic cells (e.g., insect cells, yeast cells, or mammalian cells). Alternatively, the recombinant expression vector may be transcribed and translated in vitro, for example using T7 promoter regulatory sequences and T7 polymerase.

It is understood that expression of proteins in prokaryotes may be carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. In general, in fusion expression vectors, a proteolytic cleavage site may be introduced at the junction of the fusion moiety and the recombinant protein to enable separation of the recombinant protein from the fusion moiety subsequent to purification of the fusion protein. In one example embodiment, for instance, fusion expression vectors may include, but are not limited to, pGEX, pMAL, and pRIT5, which fuse glutathione S-transferase (GST), maltose E binding protein, or protein A, respectively, to the target recombinant protein.

In one example embodiment, an artificial transcription factor disclosed herein may be expressed in a non-fusion expression vector. For instance, examples of suitable inducible non-fusion E. coli expression vectors may include, but are not limited to, pTrc and pET 11d.

If desired, to maximize recombinant transcription factor expression in E. coli, the artificial transcription factor may be expressed in a host bacterium with an impaired capacity to proteolytically cleave the recombinant protein. Alternatively, the nucleic acid sequence of the artificial transcription factor to be inserted into an expression vector may be altered (e.g., mutated) so that the individual codons for each amino acid are those preferentially utilized in E. coli. Alterations and/or mutations of nucleic acid sequences may be carried out by standard DNA synthesis techniques.

In one example embodiment, the expression vector may be a yeast expression vector. For instance, suitable yeast expression vectors may include, but are not limited to, pYepSec1, pMFa, pJRY88, pYES2, and pPicZ.

In one example embodiment, the expression vector may be a baculovirus expression vector. For instance, suitable baculovirus vectors for expression of artificial transcription factor disclosed herein in cultured insect cells (e.g., Sf 9 cells) may include, but are not limited to, the pAc series and the pVL series.

In one example embodiment, an artificial transcription factor disclosed herein may be expressed in mammalian cells using a mammalian expression vector. For instance, the mammalian expression vectors may include, but are not limited to, pCDM8 and pMT2PC.

In one example embodiment, the artificial transcription factor disclosed herein may be delivered to a host cell via a recombinant expression vector. As used herein, “host cell” and/or “recombinant host cell” refer to the particular subject cell and to the progeny or potential progeny of such a cell. Because certain modifications may occur in succeeding generations due to either mutation or environmental influences, such progeny may not, in fact, be identical to the parent cell, but are still included within the scope of the term as used herein. In some example embodiments, the host cell may be any prokaryotic (e.g., E. coli) or eukaryotic cell.

In one example embodiment, the host cell may be eukaryotic cells, e.g., insect cells, yeast cells, or mammalian cells. The mammalian cells may be, for instance, human, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like, cell lines or cell strains. Examples of such cells, cell lines or cell strains are e.g. mouse myeloma (NSO)-cell lines, Chinese hamster ovary (CHO)-cell lines, HT1080, H9, HepG2, MCF7, Jurkat T cells, MDBK Jurkat, NIH3T3, PC12, BHK (baby hamster kidney cell), VERO, SP2/0, YB2/0, Y0, C127, L cell, COS, e.g., COS1 and COS7, QC1-3, HEK-293, VERO, PER.C6, HeLA, EB1, EB2, EB3, oncolytic, or hybridoma-cell lines. In one example embodiment, the mammalian cells may be Jurkat T cells. In another example embodiment, the cell may be a CHO cell. In yet another example embodiment, the cell may be a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the Potelligent® CHOK1 SV (Lonza Biologics, Inc.). Eukaryotic cells can also be avian cells, cell lines or cell strains, such as for example, EBx® cells, EB14, EB24, EB26, EB66, or EBv13.

In one example embodiment, the eukaryotic cells may be stem cells. The stem cells may be, for instance, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs).

In one example embodiment, the cell may be a differentiated form of any of the cells described herein. In one example embodiment, the cell is a cell derived from any primary cell in culture.

In one example embodiment, the cell may be a hepatocyte such as a human hepatocyte, animal hepatocyte, or a non-parenchymal cell. For example, the cell may be a plateable metabolism qualified human hepatocyte, a plateable induction qualified human hepatocyte, plateable Qualyst Transporter Certified™ human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C57Bl/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes). Example hepatocytes are commercially available from Triangle Research Labs, LLC, 6 Davis Drive Research Triangle Park, North Carolina, USA 27709.

In one example embodiment, the eukaryotic cell may be a lower eukaryotic cell such as e.g. a yeast cell (e.g., Pichia genus (e.g., Pichia pastoris, Pichia methanolica, Pichia kluyveri, and Pichia angusta), Komagataella genus, Saccharomyces genus (e.g. Saccharomyces cerevisae, Saccharomyces kluyveri, Saccharomyces uvarum), or Kluyveromyces genus (e.g. Kluyveromyces lactis, Kluyveromyces marxianus). For instance, in one example embodiment, the eukaryotic cell may be of the species Pichia pastoris. Examples for Pichia pastoris strains may include, but are not limited to, X33, GS115, KM71, KM71H, and CBS7435.

In one example embodiment, the eukaryotic cell may be a fungal cell (e.g. Aspergillus (such as A. niger, A. fumigatus, A. orzyae, A. nidula), Acremonium (such as A. thermophilum), Chaetomium (such as C. thermophilum), Chrysosporium (such as C. thermophile), Cordyceps (such as C. militaris), Corynascus, Ctenomyces, Fusarium (such as F. oxysporum), Glomerella (such as G. graminicola), Hypocrea (such as H. jecorina), Magnaporthe (such as M. orzyae), Myceliophthora (such as M. thermophile), Nectria (such as N. heamatococca), Neurospora (such as N. crassa), Penicillium, Sporotrichum (such as S. thermophile), Thielavia (such as T. terrestris, T. heterothallica), Trichoderma (such as T. reesei), or Verticillium (such as V dahlia)).

In one example embodiment, the eukaryotic cell may be an insect cell (e.g., Sf9, Mimic™ Sf9, Sf21, High Five™ (BT1-TN-5B1-4), or BT1-Ea88 cells), an algae cell (e.g., of the genus Amphora, Bacillariophyceae, Dunaliella, Chlorella, Chlamydomonas, Cyanophyta (cyanobacteria), Nannochloropsis, Spirulina, or Ochromonas), or a plant cell (e.g., cells from monocotyledonous plants (e.g., maize, rice, wheat, or Setaria), or from a dicotyledonous plants (e.g., cassava, potato, soybean, tomato, tobacco, alfalfa, Physcomitrella patens or Arabidopsis).

In one example embodiment, the cell may be a bacterial or prokaryotic cell.

In one example embodiment, the prokaryotic cell may be Gram-positive cells such as Bacillus, Streptomyces Streptococcus, Staphylococcus, or Lactobacillus. Bacillus that may be used is, e.g., the B. subtilis, B. amyloliquefaciens, B. licheniformis, B. natto, or B. megaterium. In one example embodiment, for instance, the cell may be B. subtilis, such as B. subtilis 3NA and B. subtilis 168.

In one example embodiment, the prokaryotic cell may be a Gram-negative cell, such as Salmonella spp. or Escherichia coli (E. coli), such as e.g., TG1, TG2, W3110, DH1, DHB4, DH5a, HMS 174, HMS174 (DE3), NM533, C600, HB101, JM109, MC4100, XL1-Blue and Origami, as well as those derived from E. coli B-strains, such as BL-21 or BL21 (DE3), all of which are commercially available.

Vector DNA may be introduced into prokaryotic or eukaryotic cells via conventional transformation or transfection techniques. As used herein, “transformation” and “transfection” refer to a variety of art-recognized techniques for introducing foreign nucleic acid into a host cell, including calcium phosphate or calcium chloride co-precipitation, DEAE-dextran-mediated transfection, lipofection, electroporation, or any other suitable technique.

In some example embodiments, the activity or level of a gene of interest may be detected and/or quantified by detecting or quantifying the expressed artificial transcription factor disclosed herein. The artificial transcription factor disclosed herein may be detected and quantified by various means well-known to those of skill in the art. Aberrant levels of the artificial transcription factor disclosed herein encoded by a nucleic acid and functionally similar homologs thereof, including a fragment or genetic alteration thereof (e.g., in regulatory or promoter regions thereof) may be associated with the activation of a gene of interest. Any method known in the art for detecting nucleic acids may be used. Such methods may include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like. Binder-ligand immunoassay methods may include reacting antibodies with an epitope or epitopes and competitively displacing a labeled polypeptide or derivative thereof.

### Methods

In some example embodiments, methods disclosed herein may include treating a disease characterized by immune dysfunction by administering to the subject in need thereof an artificial transcription factor disclosed herein. The term “treating” as used herein refers to partially or completely alleviating, improving, relieving, inhibiting progression, and/or reducing incidence of one or more symptoms of a disease, disorder, and/or condition, e.g., a disease characterized by immune dysfunction.

In some example embodiments, the disease characterized by immune dysfunction may be a cancer. For instance, the cancer may include, but is not limited to, adrenal cortical cancer, advanced cancer, anal cancer, aplastic anemia, bile duct cancer, bladder cancer, bone cancer, bone metastasis, brain tumors, brain cancer, breast cancer, childhood cancer, cancer of unknown primary origin, cervical cancer, colon/rectal cancer, central nervous system (CNS) cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer (NSCLC), small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, and/or Waldenstrom macroglobulinemia. For instance, the cancer may include, but is not limited to, breast cancer, colon cancer, CNS, leukemia, melanoma, prostate, or renal cancer. In one example embodiment, the breast cancer may be triple negative breast cancer (TNBC), estrogen receptor-positive breast cancer, metastatic breast cancer, HER2 positive breast cancer, or a combination thereof. In one example embodiment, the breast cancer may be triple negative breast cancer (TNBC). In one example embodiment, the cancer may include, but is not limited to, melanoma, non-small cell lung cancer (NSCLC), triple-negative breast cancer (TNBC), renal cell carcinoma, glioblastoma, or colorectal cancer.

In some example embodiments, the disease characterized by immune dysfunction may be an autoimmune disease. For instance, in one example embodiment, the autoimmune disease may include, but is not limited to, an autoimmune rheumatologic disorder (including rheumatoid arthritis, Sjogren's syndrome, scleroderma, lupus such as SLE and lupus nephritis, polymyositis-dermatomyositis, cryoglobulinemia, anti-phospholipid antibody syndrome, and psoriatic arthritis), an autoimmune gastrointestinal and liver disorder (including inflammatory bowel diseases (e.g., ulcerative colitis and Crohn's disease), autoimmune gastritis and pernicious anemia, autoimmune hepatitis, primary biliary cirrhosis, primary sclerosing cholangitis, and celiac disease), vasculitis (including ANCA-negative vasculitis and ANCA-associated vasculitis, including Churg-Strauss vasculitis, Wegener's granulomatosis, and microscopic polyangiitis), an autoimmune neurological disorder (including multiple sclerosis, opsoclonus myoclonus syndrome, myasthenia gravis, neuromyelitis optica, Parkinson's disease, Alzheimer's disease, and autoimmune polyneuropathies), a renal disorder (including glomerulonephritis, Goodpasture's syndrome, and Berger's disease), an autoimmune dermatologic disorder (including psoriasis, urticaria, hives, pemphigus vulgaris, bullous pemphigoid, and cutaneous lupus erythematosus), a hematologic disorder (including thrombocytopenic purpura, thrombotic thrombocytopenic purpura, post-transfusion purpura, and autoimmune hemolytic anemia), atherosclerosis, uveitis, an autoimmune hearing disease (including inner ear disease and hearing loss), Behcet's disease, Raynaud's syndrome, organ transplant, or an autoimmune endocrine disorder (including diabetic-related autoimmune diseases such as insulin-dependent diabetes mellitus (IDDM), Addison's disease, and autoimmune thyroid disease (including Graves' disease and thyroiditis)). For instance, in one example embodiment, the autoimmune disease may be selected from multiple sclerosis, rheumatoid arthritis, inflammatory bowel disease, or systemic lupus erythematosus.

In some example embodiments, the disease characterized by immune dysfunction may be a chronic infectious disease. For instance, the chronic infectious disease may be selected from human immunodeficiency virus (HIV), hepatitis B virus (HBV), hepatitis C virus (HCV), human papillomavirus (HPV), high-risk HPV, or tuberculosis.

The term “subject” refers to any organism to which aspects of the disclosure can be administered, e.g., for experimental, diagnostic, prophylactic, and/or therapeutic purposes. Subjects to which embodiments of the disclosure can be administered include mammals, such as primates, for example, humans. For veterinary applications, a wide variety of subjects are suitable, e.g., livestock such as cattle, sheep, goats, cows, swine, and the like; poultry such as chickens, ducks, geese, turkeys, and the like; and domesticated animals, such as pets such as dogs and cats. For diagnostic or research applications, a wide variety of mammals are suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject.

As used herein, the term “administration” refers to introducing a substance (e.g., an artificial transcription factor disclosed herein) to a subject. The administration thereof can be carried out in any convenient manner, including by aerosol inhalation, injection, ingestion, transfusion, implantation, or transplantation. For instance, the artificial transcription factor disclosed herein may be administered orally, subcutaneously, intravenously, or intratumoral. In this regard, “oral” administration can refer to administration into a subject's mouth; “subcutaneous” administration can refer to administration just below the skin; “intravenous” administration can refer to administration into a vein of a subject; and “intratumoral” administration can refer to administration within a tumor.

Pharmaceutical compositions disclosed herein may be formulated to be compatible with its intended route of administration. As used herein, “routes of administration” may include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). The composition can be sterile and should be fluid to the extent that easy syringability exists. It can be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyethylene glycol, and suitable mixtures thereof. Prolonged absorption of the injectable compositions may be brought about by including in the composition an agent which delays absorption, for example, aluminum monostearate and gelatin.

Oral compositions may include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier may be applied orally.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Compositions for parenteral delivery, e.g., via injection, can include pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water, ethanol, polyols (e.g., glycerol, propylene glycol, polyethylene glycol and the like), carboxymethylcellulose and suitable mixtures thereof, vegetable oils (e.g., corn oil) and injectable organic esters such as ethyl oleate. In addition, the composition can contain minor amounts of auxiliary substances such as wetting or emulsifying agents, pH buffering agents and the like that can enhance the effectiveness of the phenolic compound. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents, and dispersing agents.

In one example embodiment, a therapeutically effective amount of the artificial transcription factor may be administered to the subject. The term “therapeutically effective amount” refers to those amounts that, when administered to a subject in view of the nature and severity of that subject's disease or condition, will have a desired therapeutic effect, e.g., an amount which will cure, prevent, inhibit, or at least partially arrest or partially prevent a target disease or condition. A therapeutically effective dose further may refer to that amount of the therapeutic agent sufficient to result in amelioration of symptoms, e.g., treatment, healing, prevention or amelioration of the relevant medical condition, or an increase in rate of treatment, healing, prevention, or amelioration of such conditions.

A therapeutically effective dose can depend upon a number of factors known to those of ordinary skill in the art. The dosage can vary depending upon known factors such as the pharmacodynamic characteristics of the active ingredient and its mode and route of administration; time of administration of active ingredient; identity, size, condition, age, sex, health and weight of the subject or sample being treated; nature and extent of symptoms; kind of concurrent treatment, frequency of treatment and the effect desired; and rate of excretion. These amounts can be readily determined by the skilled artisan.

In one example embodiment, the therapeutically effective amount is at least about 1 pg/mL body weight, at least about 2 pg/mL body weight, at least about 5 pg/mL body weight, at least about 10 pg/mL body weight, at least about 20 pg/mL body weight, at least about 25 pg/mL body weight, at least about 40 pg/mL body weight, at least about 50 pg/mL body weight, at least about 55 pg/mL body weight, at least about 60 pg/mL body weight, at least about 75 pg/mL body weight, at least about 100 pg/mL body weight, at least about 150 pg/mL body weight, at least about 200 pg/mL body weight, at least about 250 pg/mL body weight, at least about 300 pg/mL body weight, at least about 400 pg/mL body weight, at least about 500 pg/mL body weight.

In some example embodiments, for instance, the artificial transcription factor may be administered to a subject at a dosage of about 1 pg/mL body weight to about 100 pg/mL body weight, such as from about 5 pg/mL body weight to about 75 pg/mL body weight, such as from about 10 pg/mL body weight to about 60 pg/mL body weight, or any range therebetween. For instance, in one example embodiment, the artificial transcription factor may be administered to a subject at a dosage of about 1 pg/mL body weight to about 100 pg/mL body weight. In another example embodiment, the artificial transcription factor may be administered to a subject at a dosage of about 5 pg/mL body weight to about 75 pg/mL body weight. In another example embodiment, the artificial transcription factor may be administered to a subject at a dosage of about 10 pg/mL body weight to about 60 pg/mL body weight.

### Regulating Genes

In some example embodiments, methods disclosed herein may include modulating expression of a gene of interest by administering an expression vector expressing a nucleic acid encoding an artificial transcription factor disclosed herein to a host cell. For instance, the artificial transcription factor disclosed herein may modulate expression of a gene of interest in a cell in vitro, in vivo, or ex vivo. For instance, methods may include activating expression of an immune signaling gene in a cell. In one example embodiment, the artificial transcription factor disclosed herein may upregulate a gene of interest in a cell. For instance, the artificial transcription factor disclosed herein may upregulate immune signaling genes.

In some example embodiments, methods may include activating expression of an immune signaling gene in a cell. For instance, in one example embodiment, methods for activating expression of an immune signaling gene in a cell may include contacting the cell with an artificial transcription factor disclosed herein. For instance, in one example embodiment, the zinc finger proteins of the artificial transcription factor disclosed herein may bind to a target site to mediate transcriptional activation.

In one example embodiment, the zinc finger proteins may bind to a target site corresponding to an amino acid sequence set forth in SEQ ID NOs: 5-28 and/or 30-34, or a functionally equivalent variant thereof to mediate transcriptional activation. For instance, in one example embodiment, the zinc finger proteins may bind to one or more target sites corresponding to an amino acid sequence set forth in SEQ ID NOs: 5-28 and/or 30-34, or a functionally equivalent variant thereof. For instance, the functionally equivalent variant may be at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to an amino acid sequence set forth in SEQ ID NOs: 5-28 and/or 30-34. In one example embodiment, the functionally equivalent variant may be at least 70% identical to an amino acid sequence set forth in SEQ ID NOs: 5-28 and/or 30-34. In another example embodiment, the functionally equivalent variant may be at least 80% identical to an amino acid sequence set forth in SEQ ID NOs: 5-28 and/or 30-34. In another example embodiment, the functionally equivalent variant may be at least 85% identical to an amino acid sequence set forth in SEQ ID NOs: 5-28 and/or 30-34. In another example embodiment, the functionally equivalent variant may be at least 90% identical to an amino acid sequence set forth in SEQ ID NOs: 5-28 and/or 30-34. In another example embodiment, the functionally equivalent variant may be at least 95% identical to an amino acid sequence set forth in SEQ ID NOs: 5-28 and/or 30-34. In another example embodiment, the functionally equivalent variant may be at least 98% identical to an amino acid sequence set forth in SEQ ID NOs: 5-28 and/or 30-34.

In one example embodiment, the zinc finger proteins may bind to a target site corresponding to a nucleotide sequence set forth in SEQ ID NOs: 71-75, or a functionally equivalent variant thereof to mediate transcriptional activation. For instance, in one example embodiment, the zinc finger proteins may bind to one or more target sites corresponding to a nucleotide sequence set forth in SEQ ID NOs: 71-75, or a functionally equivalent variant thereof. For instance, the functionally equivalent variant may be at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a nucleotide sequence set forth in SEQ ID NOs: 71-75. In one example embodiment, the functionally equivalent variant may be at least 70% identical to a nucleotide sequence set forth in SEQ ID NOs: 71-75. In another example embodiment, the functionally equivalent variant may be at least 80% identical to a nucleotide sequence set forth in SEQ ID NOs: 71-75. In another example embodiment, the functionally equivalent variant may be at least 85% identical to a nucleotide sequence set forth in SEQ ID NOs: 71-75. In another example embodiment, the functionally equivalent variant may be at least 90% identical to a nucleotide sequence set forth in SEQ ID NOs: 71-75. In another example embodiment, the functionally equivalent variant may be at least 95% identical to a nucleotide sequence set forth in SEQ ID NOs: 71-75. In another example embodiment, the functionally equivalent variant may be at least 98% identical to a nucleotide sequence set forth in SEQ ID NOs: 71-75.

In one example embodiment, the zinc finger proteins may bind to a target site corresponding to an amino acid sequence set forth in SEQ ID NOs: 43-66 and/or 67-70, or a functionally equivalent variant thereof to mediate transcriptional activation. For instance, in one example embodiment, the zinc finger proteins may bind to one or more target sites corresponding to an amino acid sequence set forth in SEQ ID NOs: 43-66 and/or 67-70, or a functionally equivalent variant thereof. For instance, the functionally equivalent variant may be at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to an amino acid sequence set forth in SEQ ID NOs: 43-66 and/or 67-70. In one example embodiment, the functionally equivalent variant may be at least 70% identical to an amino acid sequence set forth in SEQ ID NOs: 43-66 and/or 67-70. In another example embodiment, the functionally equivalent variant may be at least 80% identical to an amino acid sequence set forth in SEQ ID NOs: 43-66 and/or 67-70. In another example embodiment, the functionally equivalent variant may be at least 85% identical to an amino acid sequence set forth in SEQ ID NOs: 43-66 and/or 67-70. In another example embodiment, the functionally equivalent variant may be at least 90% identical to an amino acid sequence set forth in SEQ ID NOs: 43-66 and/or 67-70. In another example embodiment, the functionally equivalent variant may be at least 95% identical to an amino acid sequence set forth in SEQ ID NOs: 43-66 and/or 67-70. In another example embodiment, the functionally equivalent variant may be at least 98% identical to an amino acid sequence set forth in SEQ ID NOs: 43-66 and/or 67-70.

In one example embodiment, the zinc finger proteins may bind to a target site corresponding to a nucleotide sequence set forth in SEQ ID NOs: 76-79, or a functionally equivalent variant thereof to mediate transcriptional activation. For instance, in one example embodiment, the zinc finger proteins may bind to one or more target sites corresponding to a nucleotide sequence set forth in SEQ ID NOs: 76-79, or a functionally equivalent variant thereof. For instance, the functionally equivalent variant may be at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a nucleotide sequence set forth in SEQ ID NOs: 76-79. In one example embodiment, the functionally equivalent variant may be at least 70% identical to a nucleotide sequence set forth in SEQ ID NOs: 76-79. In another example embodiment, the functionally equivalent variant may be at least 80% identical to a nucleotide sequence set forth in SEQ ID NOs: 76-79. In another example embodiment, the functionally equivalent variant may be at least 85% identical to a nucleotide sequence set forth in SEQ ID NOs: 76-79. In another example embodiment, the functionally equivalent variant may be at least 90% identical to a nucleotide sequence set forth in SEQ ID NOs: 76-79. In another example embodiment, the functionally equivalent variant may be at least 95% identical to a nucleotide sequence set forth in SEQ ID NOs: 76-79. In another example embodiment, the functionally equivalent variant may be at least 98% identical to a nucleotide sequence set forth in SEQ ID NOs: 76-79.

In one example embodiment, the artificial transcription factor disclosed herein may recognize a target binding site that is at least 3 bp, 6 bp, 9 bp, 12 bp, 15 bp, 18 bp, 21 bp, 24 bp, 27 bp, 30 bp, 33 bp, or 36 bp in size. For instance, the artificial transcription factor disclosed herein may recognize a target binding site that is more than 3 bp, 6 bp, 9 bp, 12 bp, 15 bp, 18 bp, 21 bp, 24 bp, 27 bp, or 30 bp. In one example embodiment, the artificial transcription factor disclosed herein may recognize a target binding site that is from 9-33 bp, 9-30 bp, 9-27 bp, 9-24 bp, 9-21 bp, 9-18 bp, 9-15 bp, 9-12 bp, 12-33 bp, 12-30 bp, 12-27 bp, 12-24 bp, 12-21 bp, 12-18 bp, 12-15 bp, 15-33 bp, 15-30 bp, 15-27 bp, 15-24 bp, 15-21 bp, 15-18 bp, 18-33 bp, 18-30 bp, 18-27 bp, 18-24 bp, 18-21 bp, 21-33 bp, 21-30 bp, 21-27 bp, 21-24 bp, 24-33 bp, 24-30 bp, 24-27 bp, 27-33 bp, 27-30 bp, or 30-33 bp. For instance, in one example embodiment, the artificial transcription factor disclosed herein that upregulates a gene of interest may recognize a target binding site that may be from 18-27 bp.

In one example embodiment, an artificial transcription factor disclosed herein that upregulates a gene of interest may result in at least a 1.5 fold, 2 fold, 3 fold, 4 fold, 5 fold, 6 fold, 7 fold, 8 fold, 9 fold, 10 fold, 15 fold, 20 fold, 25 fold, 50 fold, 100 fold, or greater upregulation of gene expression in a cell or in vivo compared to a control (e.g., no artificial transcription factor or a transcription factor that does not recognize the target site). In another example embodiment, an artificial transcription factor disclosed herein that upregulates a gene of interest may result in at least a 50%, 60%, 70%, 75%, 80%, 90%, 100%, 125%, 150%, 200%, 250%, 300%, 400%, or 500% or greater upregulation of gene expression in a cell or in vivo compared to a control (e.g., no artificial transcription factor or a transcription factor that does not recognize the target site).

In one example embodiment, an artificial transcription factor disclosed herein that upregulates a gene of interest binding to a target site that is capable of increasing said gene expression by at least 1.2 fold, 1.3 fold, 1.4 fold, 1.5 fold, 1.6 fold, 1.7 fold, 1.8 fold, 1.9 fold, 2 fold, 3 fold, 4 fold, 5 fold, 8 fold, 10 fold, 12 fold, 15 fold, 18 fold, 20 fold, 25 fold, 30 fold, 40 fold, 50 fold, 75 fold, 100 fold, or greater relative to a control in a transcriptional activation assay. In another example embodiment, an artificial transcription factor disclosed herein that upregulates a gene of interest binds to a target site that is capable of increasing said gene expression by at least 20%, 30%, 40%, 50%, 60%, 70%, 75%, 80%, 90%, 100%, 125%, 150%, 200%, 250%, 300%, 400%, 500% or greater relative to a control in a transcriptional activation assay.

In some example embodiments, an artificial transcription factor disclosed herein may upregulate the expression of a cytokine. As used herein, a “cytokine” refers to any of numerous small, secreted proteins that act on a cell population as intercellular mediators. For example, cytokines regulate the intensity and duration of the immune response by affecting an immune cell's differentiation process, which involves changes in gene expression by which a precursor cell becomes a distinct specialized cell type. Cytokines have been variously named as lymphokines, interleukins, and chemokines, based on their function, cell of secretion, or target of action.

In one example embodiment, cytokines may include, but are not limited to, lymphokines, monokines, and traditional polypeptide hormones. For instance, cytokines upregulated by an artificial transcription factor disclosed herein may include, but are not limited to, interferons (IFN, such as IFN-γ), interleukins (IL, notably IL-1, IL-2, IL-4, IL-10, IL-12, IL-15, IL-18, IL-13), a stimulator of interferon genes (STING), GMP-AMP Synthase (GAS), signal transducer and activator of transcription (STAT), nuclear factor kappa B (NF-kB), colony stimulating factors (CSF), thrombopoietin (TPO), erythropoietin (EPO), leukemia inhibitory factor (LIF), kit-ligand, growth hormones (GH), insulin-like growth factors (IGF), parathyroid hormone, thyroxine, insulin, relaxin, follicle stimulating hormone (FSH), thyroid stimulating hormone (TSH), leutinizing hormone (LH), hematopoietic growth factor, hepatic growth factor, fibroblast growth factors (FGF), prolactin, placental lactogen, tumor necrosis factors (TNF), such as TNFα, mullerian-inhibiting substance, mouse gonadotropin-associated peptide, inhibin, activin, vascular endothelial growth factor (VEGF), integrin, nerve growth factors (NGF), platelet growth factor, transforming growth factors (TGF), such as TGF-β osteoinductive factors, etc. For instance, in some example embodiments, an artificial transcription factor disclosed herein may upregulate the expression of an immune signaling gene including, but is not limited to, interleukin-2 (IL-2), IL-7, IL-12, IL-15, IL-21, interferon gamma (IFN-γ), tumor necrosis factor alpha (TNF-α), tumor necrosis factor beta (TNF-β), or granulocyte-macrophage colony-stimulating factor (GM-CSF). In one example embodiment, an artificial transcription factor disclosed herein may upregulate the expression of IFN-γ. In another example embodiment, an artificial transcription factor disclosed herein may upregulate the expression of IL-1, IL-2, IL-7, IL-12, IL-15, IL-21, or a combination thereof. In yet another example embodiment, an artificial transcription factor disclosed herein may upregulate the expression of TNF-α. In yet another example embodiment, an artificial transcription factor disclosed herein may upregulate the expression of TNF-β. In yet another example embodiment, an artificial transcription factor disclosed herein may upregulate the expression of GM-CSF. In yet another example embodiment, an artificial transcription factor disclosed herein may upregulate the expression of a stimulator of interferon genes (STING). In yet another example embodiment, an artificial transcription factor disclosed herein may upregulate the expression of a GMP-AMP Synthase (GAS). In yet another example embodiment, an artificial transcription factor disclosed herein may upregulate the expression of a signal transducer and activator of transcription (STAT). In yet another example embodiment, an artificial transcription factor disclosed herein may upregulate the expression of a nuclear factor kappa B (NF-kB).

In some example embodiments, an artificial transcription factor disclosed herein may upregulate an immune-related signaling protein. For instance, the immune-related signaling protein may include, but is not limited to, interleukin (IL)-4, IL-10, IL-12, granulocyte-macrophage colony-stimulating factor (GM-CSF), FMS-related tyrosine kinase 3 (FLT3), and C-C chemokine receptor type 7 (CCR7). For instance, in one example embodiment, an artificial transcription factor disclosed herein may upregulate IL-4, IL-10, and/or IL-12. In another example embodiment, an artificial transcription factor disclosed herein may upregulate granulocyte-macrophage colony-stimulating factor (GM-CSF). In yet another example embodiment, an artificial transcription factor disclosed herein may upregulate FMS-related tyrosine kinase 3 (FLT3). In yet another example embodiment, an artificial transcription factor disclosed herein may upregulate C-C chemokine receptor type 7 (CCR7).

In some example embodiments, an artificial transcription factor disclosed herein may be in a pharmaceutical composition, which include but are not limited to DNA- or RNA-encoded vectors, lipid nanoparticles, viral capsids, recombinant proteins, synthetic proteins, protein conjugates, or antibody conjugates. Pharmaceutical compositions for parenteral, intradermal, or subcutaneous injection can include pharmaceutically acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include, but are not limited to, water, ethanol, polyols (e.g., glycerol, propylene glycol, polyethylene glycol and the like), carboxymethylcellulose and suitable mixtures thereof, vegetable oils (e.g., olive oil), and injectable organic esters such as ethyl oleate. A pharmaceutical composition can contain minor amounts of auxiliary substances such as wetting or emulsifying agents, pH buffering agents and the like that can enhance the effectiveness of the active ingredient. Proper fluidity may be maintained, for example, by the use of coating materials such as lecithin, by the maintenance of the required particle size in the case of dispersions, and by the use of surfactants. A pharmaceutical composition may also contain adjuvants such as preservatives, wetting agents, emulsifying agents, and dispersing agents. It may also be desirable to include isotonic agents such as sugars, sodium chloride and the like.

In some example embodiments, methods disclosed herein may be directed to delivering a pharmaceutical composition including artificial transcription factors disclosed herein to a cell. For instance, the delivery of artificial transcription factors disclosed herein may be via transfection or electroporation of the artificial transcription factors disclosed herein as one or more nucleic acid molecules that are expressed in the cell and delivered to the surface of the cell. In one example embodiment, for instance, a vector encoding an artificial transcription factor protein disclosed herein may be delivered to a subject with electroporation, lipofectamine, lentiviral vectors, spinnoculation, liposome mediated, nanoparticle facilitated, and/or recombinant vectors. For instance, in one example embodiment, recombinant artificial transcription factor proteins may be delivered with cell-penetrating peptides, immunotoxins, lipofectamine, and/or other methods well-known in the art. In some example embodiments, delivery constructs may be targeted to specific cell types or tissues including, but is not limited to, Nk cells, cytotoxic (CD8+) and immune-memory (CD4+) T cells, B cells (B220/CD45R+), monocytes/macrophages (CD11b+), dendritic cells (CD11c+), Her2 positive or negative cancer cells, or a combination or subpopulation thereof.

The preceding description is exemplary in nature and is not intended to limit the scope, applicability or configuration of the disclosure in any way. Various changes to the described embodiments may be made in the function and arrangement of the elements described herein without departing from the scope of the disclosure.

Here and throughout the specification and claims, range limitations are combined and interchanged, such ranges are identified and include all the sub-ranges contained therein unless context or language indicates otherwise. For example, all ranges disclosed herein are inclusive of the endpoints, and the endpoints are independently combinable with each other.

This written description uses examples to disclose the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

It is to be understood that the terminology used herein is for the purpose of describing particular embodiments only and is not intended to limit the scope of the present disclosure. In this specification and in the claims that follow, reference will be made to a number of terms that shall be defined to have the following meanings.

Unless defined otherwise, all technical and scientific terms used herein have the same meaning as commonly understood by one of ordinary skill in the art to which this invention is related.

As used in this application and in the claims, the singular forms “a”, “an”, and “the” include the plural forms unless the context clearly dictates otherwise. Additionally, the term “includes” means “comprises”. The methods and compositions of the present disclosure, including components thereof, can comprise, consist of, or consist essentially of the essential elements and limitations of the embodiments described herein, as well as any additional or optional ingredients, components or limitations described herein or otherwise useful in biocidal compositions.

Unless otherwise indicated, all numbers expressing quantities of ingredients, properties such as molecular weight, percentages, and so forth, as used in the specification or claims are to be understood as being modified by the term “about”. Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximate unless the word “about” is recited.

As used herein, “optional” or “optionally” means that the subsequently described material, event or circumstance may or may not be present or occur, and that the description includes instances where the material, event or circumstance is present or occurs and instances in which it does not. As used herein, “w/w %” and “wt %” mean by weight as relative to another component or a percentage of the total weight in the composition.

The term “about” is intended to mean approximately, in the region of, roughly, or around. When the term “about” is used in conjunction with a numerical range, it modifies that range by extending the boundaries above and below the numerical values set forth. Unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

The phrase “effective amount” means an amount of a compound that promotes, improves, stimulates, or encourages a response to the particular condition or disorder or the particular symptom of the condition or disorder.

Furthermore, certain aspects of the present disclosure may be better understood according to the following examples, which are intended to be non-limiting and exemplary in nature. Moreover, it will be understood that the compositions described in the examples may be substantially free of any substance not expressly described.

The present disclosure may be better understood with reference to the following examples.

### EXAMPLES

### Materials and Methods

### Construction of Artificial Transcription Factor Proteins

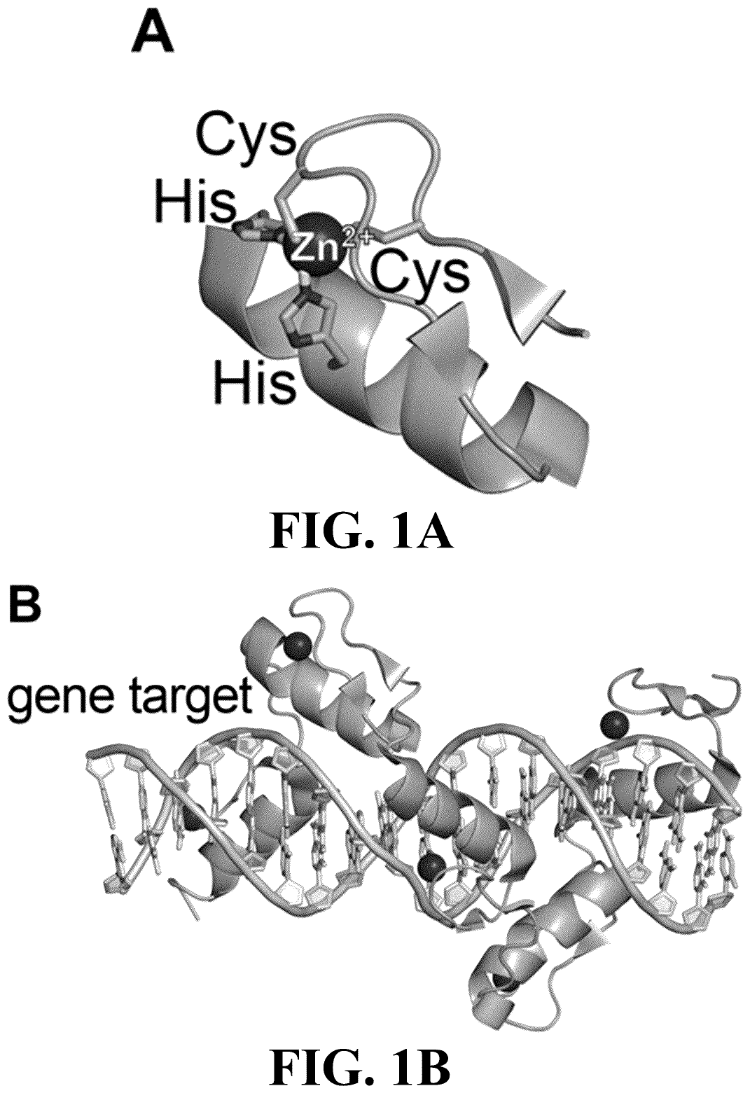

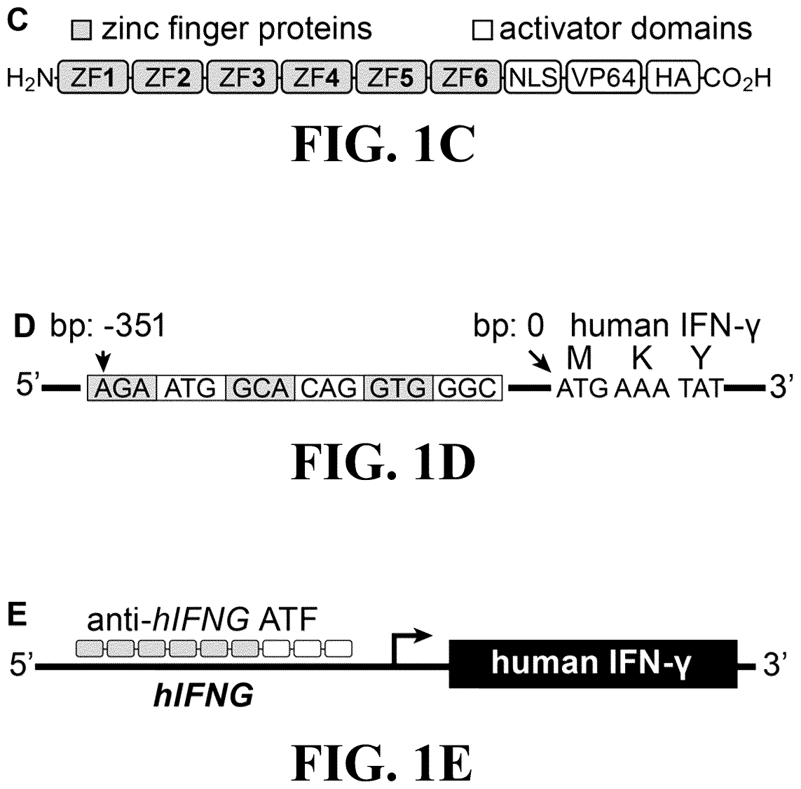

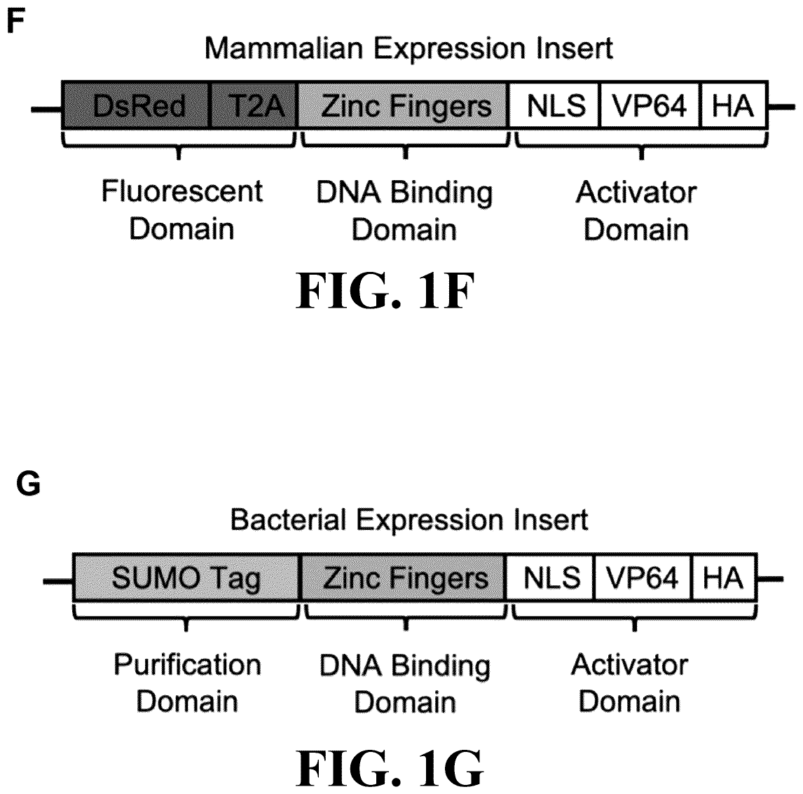

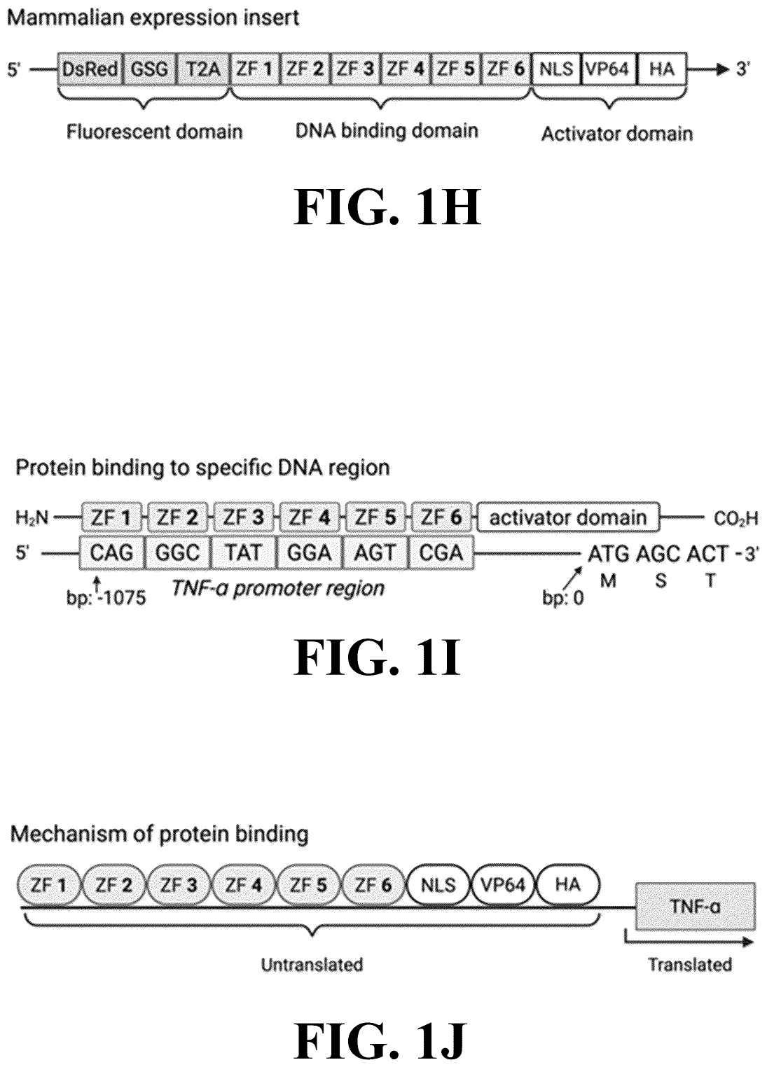

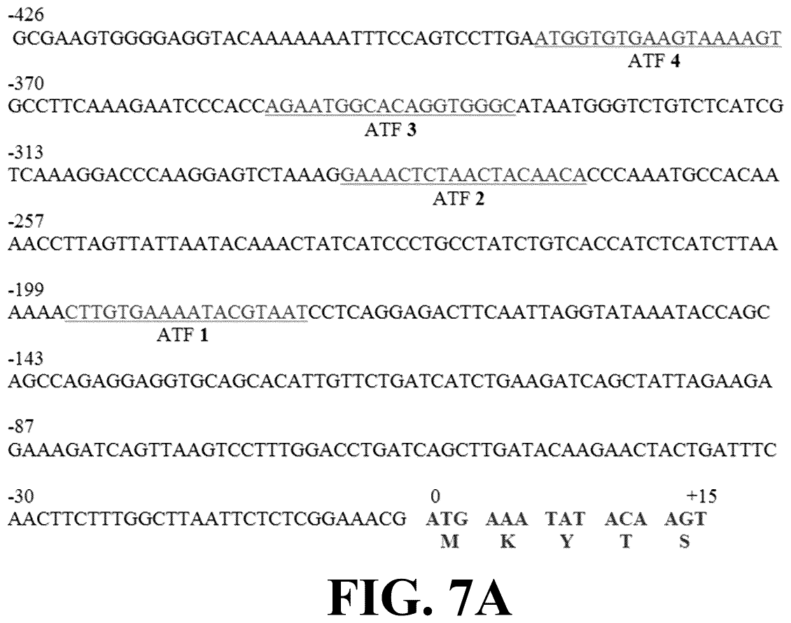

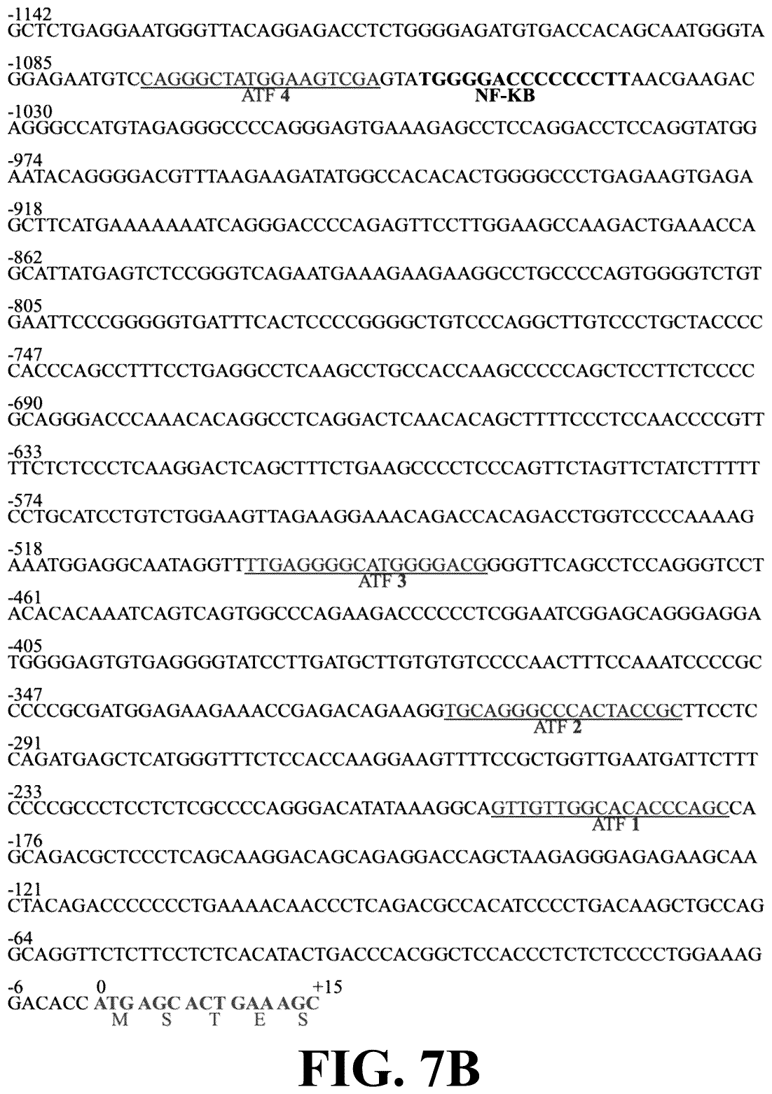

To identify IFNG gene regions for activating IFN-γ expression, human chromosome 12 was downloaded from the National Center for Biotechnology Information (NCBI, Gene ID: 3,458, annotation release: RS_2023_10). The targeted gene includes the 500 base pairs (bp) upstream of the first exon, which was imported into the ZFTools website to assist with identifying gene binding locations of the zinc finger (ZF) protein. The program was adjusted to identify 18 bp sequences that the ZF proteins could recognize through fusion proteins containing six individual ZF subunit proteins (FIGS. 1C-1D). Four sets of ZF proteins were selected that bind throughout the IFN-γ promoter region (FIG. 7A; Tables 1-3).

Additionally, the targeted TNF gene includes 1200 base pairs (bp) upstream of the first exon. Four sets of codons 18 bp in length were selected to generate four TNF-Targeting ATF proteins 1-4 (FIG. 7B). Zinc finger proteins (ZFPs) recognize and bind to a 3 bp region of DNA The TNF consruct included a red fluorescence protein, dsRed, a Glycine-Serine-Glycine linker, a self-cleaving peptide, T2A, a nuclear localization signal and a VP64 co-activator (FIGS. 1H and 1J.

### Mammalian ATF Expression Plasmids

Genetic sequences for ATFs 1-4 were optimized for homo sapien codons and clonally inserted into a mammalian expression plasmid (pTwist CMV Puro, Twist Biosciences). A plasmid encoding only the DsRed fluorescent protein was also prepared as a transfection control. Another plasmid encoding a DsRed-Aart6 fusion protein was also prepared as an isotype control. The mammalian expression plasmids were transformed into E. coli (DH5-α, cat. no. C2987, New England Biosciences), expanded overnight in 1000-mL cultures at 37° C. with Luria-Bertani (LB) broth and carbenicillin (100 μg/mL), and isolated by centrifugation. The cultures were subdivided for purification by the ZymoPURE II Maxiprep kit (Zymo Research). Plasmids were eluted from the membrane with 2×200 μL (400 μL total) of ultrapure water (18 MΩ), which had been pre-filtered (0.4 μm) and warmed to 37° C. Isolated plasmids were further purified to remove endotoxin and stored at −20° C. DNA concentrations were determined based on the optical density at 260 nm using a SpectraDrop microplate (0.5 mm path length) and a UV-vis plate reader (SpectraMax iD3). The recombinant plasmids typically yielded ≥3 mg of rDNA per 1,000 mL of culture.

### Mammalian Cell Culture

Human Jurkat cells (ATCC, TIB-152) were cultured in Roswell Park Memorial Institute Medium (RPMI) containing GlutaMax supplemented with 10% v/v fetal bovine serum (FBS, Gibco) and 100 U/mL penicillin-streptomycin (Cytiva) at 37° C. in 5% v/v CO₂ (g).

Plasmid Transfection into Jurkat Cells

ATF 1-4 plasmids were transfected into Jurkat T cells using a Bio-Rad Gene Pulser Xcell Electroporator. Before electroporation, the cells were grown to 80% confluency, collected by centrifugation (1,000 g×5 min), manually counted on a hemacytometer, and resuspended to give a working stock solution of 5×10⁶ cells/mL in RPMI (10% FBS+Pen/Strep). To a 2 mm MicroPulser cuvette (Bio-Rad), 1×10⁶ Jurkat cells were added with 100 μg of plasmid in RPMI to reach a total volume of 200 μL. After gently mixing the suspension with a pipette, the transfection was performed using an exponential decay pulse; capacitance=1,000 μF and voltage=140 V. Four sets of electroporated cells (200 μL) were pooled into one well of a 6-well plate with complete RPMI media (final volume=2 mL) and equilibrated for 1 h at 37° C. and 5% C02. The cells were further incubated in the presence of 10 μM ZnCl₂, 1× phorbol 12-Myristate 13-Acetate (PMA)/ionomycin, and 3 μg/mL brefeldin A. At the indicated time points, the cells were processed and analyzed.

### Fluorescence Microscopy

Transfection efficiency was determined using fluorescence microscopy based on the fraction of live cells expressing the DsRed (λ_{Ex}=558 nm, λ_{Em}=583 nm) protein. Cells were prepared for microscopy by transferring the cell suspensions into microcentrifuge tubes (1.7 mL), pelleting by centrifugation (1,000×g, 5 min), and resuspending in PBS. Live-cell staining was achieved by incubating the cells with the Calcein AM viability dye (Invitrogen), pelleting by centrifugation, and resuspending in PBS. Microscopy images of Calcein AM (λ_{Ex}=494 nm, λ_{EM}=517 nm) were obtained at 5× and 10× magnification and an exposure time of 300 msec. Images of DsRed-expressing cells were obtained at 5× and 10× magnification with an exposure time of 6.53 s. Transfection efficiencies were calculated using NIH ImageJ. Scale bars were calculated using the 1951 United States Air Force resolution test chart.

### Enzyme-Linked Immunosorbent Assay (ELISA)

A sandwich ELISA protocol enabled the measurement of IFN-γ expression from Jurkat cells. High-binding 96-well plates (Corning) were treated with 100 μL of purified anti-human IFN-γ (0.5 mg/mL, BioLegend) and incubated at 4° C. for 16 h. The wells were washed with PBS containing 0.05% Polysorbate-20 (3×100 μL) and blocked for 1 h with 200 μL of 1× the “Assay Diluent” buffer (BioLegend). Intracellular protein was isolated from the Jurkat cells after treatment with RIPA lysis buffer, quantified using a Bradford assay, diluted to 5 mg/mL concentration in the Assay Diluent buffer, and loaded in triplicate to the 96-well plate (100 μL each). Recombinant human IFN-γ (BioLegend) protein was also titrated into the 96-well plate to generate the standard curve, which ranged in concentration from 0 to 500 pg/mL. The plates were incubated for 2 h and washed with PBST (3×100 μL). Biotin anti-human IFN-γ (100 μL, 0.5 mg/mL, BioLegend) was added to the wells, incubated for 1 h, and washed with PBST (3×100 μL). HRP-Avidin (BioLegend) was added to the wells (100 μL), incubated for 30 min, and washed with PBST (3×10 μL). TMB substrate (BioLegend) was added to the wells (50 μL), incubated for 15 min, and quenched with 100 μL of stop solution (BioLegend). The absorbance of the wells was recorded at 450 nm, IFN-γ concentrations were calculated in Excel based on a standard curve with 0-500 pg/mL of IFN-γ protein. The experiment results were plotted and analyzed in GraphPad Prism. Statistical significance was evaluated using ANOVA with Holm-Sidak multiple comparisons test (*p<0.05; **p<0.005; ***p<0.001 and ****p<0.0001).

### RNA Sequencing Experiments

Cells obtained from the treatment conditions were processed using a RNeasy Minikit (Qiagen) and RNase-free DNase (Qiagen). The total RNA was collected in nuclease-free water and stored at −80° C. until further analysis. The RNA was reverse-transcribed into double-stranded cDNA libraries by poly(A) selection using a NEBNext Ultra II RNA Library Prep Kit for Illumina (New England Biolabs, Ipswich, MA, United States), and sequenced using paired-end short-read sequencing (2×150 bp) on an Illumina platform (Azenta/Genewiz).

### RNA-Seq Data Preprocessing

FASTQ files containing raw paired-end sequencing reads were processed using the CLC Genomics Workbench 24.0.2. Quality trimming steps included the removal of adapter sequences detected in reads. Reads with more than two ambiguous nucleotides were trimmed; sequences shorter than six nucleotides were discarded. Trimming also addressed low-quality sequences with a quality limit of 0.05. The resulting trimmed reads maintained an average length between 144.83 and 147.29 bases across samples. Approximately 13.9% of read pairs were trimmed due to adapter contamination. The final number of genes in the expression profile of all samples was 24,493.

The data analysis pipeline script was written in RStudio to analyze RNA-seq differential gene expression results; processing, statistical analysis, and visualization were conducted using specificR packages. The analysis required a raw gene expression counts matrix downloaded from the CLC Genomics Workbench and a metadata table containing sample-specific treatment information. Both files were uploaded in CSV format. A validation step ensured the metadata sample names matched the appropriate column names in the count matrix. The count matrix was reordered to align with the metadata as a reactive value for differential expression analysis. Raw fastq files of the data are available from the Gene Expression Omnibus (GEO) database under accession number GSE284420.

### Differential Expression Analysis

Differential expression analysis was performed using the DESeq2 package, which models count data using a negative binomial generalized linear model. The DESeq ( ) function was used to estimate normalization factors, dispersion, and model parameters. Log 2 fold changes and associated statistics (standard errors, p-values, adjusted p-values) were calculated in redefined contrasts against the two controls, Aart6 (isotype control) and shock (vehicle control). Wald tests were used for hypothesis testing, and adjusted p-values were calculated using the False Discovery Rate (FDR) method. Results for specific contrasts were extracted and exported as CSV files for further analysis and visualization.

### Data Visualization

Principal Component Analysis (PCA) was used to explore variance structures in the gene expression data. Before PCA, genes with zero or constant variance were filtered, and the data was standardized. The analysis was performed using the prcomp ( ) function, and the first three principal components were visualized in an interactive 3D scatterplot created with the plotly package. Volcano plots provided a graphical representation of the relationship between log 2 fold changes and statistical significance, highlighting genes that surpassed defined thresholds (log 2 fold change >1 or <−1, adjusted p-value<0.05), as indicated by vertical lines. Similarly, MA plots visualized the relationship between mean expression levels and log 2 fold changes, with fold change thresholds (log 2 fold change>1 or <−1, adjusted p-value<0.05) indicated by horizontal lines. Scatterplots were employed to compare test statistics between the experimental conditions, and genes meeting significance criteria (T-value>10 or <−10, on each axis) were highlighted and labeled. Heatmaps, generated using the pheatmap package, displayed log 2-transformed expression levels of selected genes averaged across treatment groups, with row-wise scaling to emphasize relative expression changes. All visualizations were exported as PDFs. Boxplots of gene expression were generated using r log-transformed data from DESeq2 results. Selected genes were used to subset the transformed expression matrix, and the data were combined with metadata for plotting. Boxplots were set by gene and included jittered sample points, with expression grouped by treatment.

### SDS-PAGE

Prior to electrophoresis, samples were mixed with loading dye, boiled (95° C., 5 min), and loaded into a polyacrylamide gradient (4%-16%) gel equilibrated with a Tris-Glycine SDS running buffer (Invitrogen). Gels were electrophoresed at 200 V for 40 min, visualized by Coomassie stain, and imaged using an Azure 600 Imaging System. Protein molecular weights were compared to a SeeBlue Plus2 (Invitrogen) molecular weight ladder.

### Mass Spectrometry

Protein identity was confirmed by analytical LC-MS using an Agilent 1260 RP-HPLC coupled to an Agilent Q-TOF 6545XT. Proteins (100 ng) were injected from a 0.1 mg/mL sample of 50% CH₃CN in H₂O with 1% formic acid (FA). Samples were eluted with a gradient of 1%-61% CH₃CN over 10 min on an Agilent Poroshell C18 column with a 0.5 mL/min flow rate. Spectra were recorded under positive ionization mode with an extended dynamic range (2 GHz) and standard mass range (m/z from 300 to 3,000).

### Bacterial ATF Expression Plasmids

The genetic sequence for SUMO-ATF 3r was designed as a fusion protein (Table 6) and codon-optimized for recombinant E. coli expression (Table 7). The gene was clonally inserted into a pET-21 (+) bacterial expression plasmid (Twist Biosciences). The plasmid was transformed into E. coli (BL21, New England Biolabs), expanded into 1 L of LB with carbenicillin (100 μg/mL), and grown (37° C., 180 rpm) until an optical density (OD₆₀₀) of 0.6-0.8 was reached. Protein expression was induced by the addition of IPTG (final conc.=0.4 mM), and allowed to proceed overnight (30° C., 150 rpm). The cultures were isolated by centrifugation (20 min×4,000 rpm) in 500-mL polypropylene centrifuge tubes (Corning), transferred to a 50-mL conical tube, and stored at −80° C. as frozen pellets.

The recombinant protein was purified after thawing and processing the frozen pellets. Pellets were resuspended in Tris-buffered saline (100 mM Tris, 500 mM NaCl, pH=7.4) supplemented with an EDTA-free protease inhibitor and lysozyme (10 pg/mL). Cell suspensions were lysed by sonication and fractionated by centrifugation (12,100 rpm, 30 min). The supernatant was loaded onto a 5-mL Ni NTA (FF, Cytiva) column for affinity-based purification. Protein was eluted using a linear elution gradient with an imidazole buffer (10 mM Tris, 150 mM NaCl, and 500 mM imidazole, pH=7.4). Samples at each stage of purification were collected for analysis by SDS-PAGE and LC-MS.

### Protein Cleavage and Purification

ATF 3r was obtained after cleavage of the fusion protein. SUMO-ATF 3r was transferred to tris-buffered saline (TBS) to ensure the removal of residual imidazole, treated with 1% Ulp-1 (w/w), incubated at 30° C., and monitored by LC-MS. ATF 3r was purified on a Ni NTA column to remove the SUMO tag and Ulp-1. The identity of ATF 3r was confirmed by SDS-PAGE and LC-MS.

### Preparative RP-HPLC

ATF 3r protein was dissolved in a solution of 20% acetonitrile in water with 0.1% TFA, pushed through a syringe filter (Nylon, 0.22 μm), and purified by RP-HPLC using an Agilent 1,290 Infinity II Preparative Open-Bed Sampler/Collector. Protein was loaded onto an Agilent Prep-C18 column (21.2×50 mm, 5 μm particle size) at a flow rate of 15 mL/min, and eluted with a 10%-40% gradient (1% per minute) of solvent B in A (solvent A: water, 0.1% TFA; solvent B: acetonitrile, 0.1% TFA). Fractions of the eluted protein were collected and analyzed by LC-MS. Fractions containing the purified protein were pooled and lyophilized.

### Protein Refolding

Lyophilized ATF 3r protein was resuspended at 1 mg/mL in 10 mM HEPES buffer at pH 7.4. The solution was dialyzed with 10 mM HEPES to remove residual TFA. The cysteine residues were reduced by incubating with 6.67 mM TCEP (from a 20 mM stock solution) at 37° C. for 1 h. The protein was subjected to refolding conditions by adding zinc acetate to a final concentration of 10 mM, followed by further incubation at 37° C. for 1 h. The folded protein was obtained after dialysis with 10 mM HEPES to remove excess TCEP and zinc acetate.

### Circular Dichroism (CD)

CD spectra of ATF 3r were recorded in a 1-mm quartz cuvette on a J-1500 Jasco spectrometer at 185-260 nm (20 nm/min). Protein samples were prepared at 35 mM in 10 mM HEPES buffer at pH 7.4 and allowed to equilibrate to room temperature before analysis. Data were collected in triplicate and the signal was averaged to give the final CD spectrum.

### Differential Scanning Fluorimetry

Thermal stability of ATF 3r was determined by heat denaturation experiments in the presence of a fluorophore. The protein was suspended at a concentration of 1 mg/mL in 10 mM HEPES (pH 7.4) and treated with an equal amount of SYPRO Orange (10×). Samples were evaluated on a Bio-Rad thermocycler by increasing the temperature with a linear ramp from 25° C. to 90° C. (rate=0.5° C. per min), collecting simultaneous fluorescence measurements of SYPRO Orange (λ_{Ex}=491 nm, λ_{Em}=586 nm). Data were background-subtracted in Excel and plotted in GraphPad Prism.

### Electrophoretic Mobility Shift Assay (EMSA)

DNA recognition of ATF 3r was evaluated upon mixing the protein with the target DNA sequence. Mixtures of protein and DNA were prepared at a fixed total volume (20 μL) and DNA concentration (20 pmol), but with increasing equivalents of the ATF 3r protein (0-20).

The sample buffer comprised 2.5% glycerol, 5 mM MgCl₂, 0.05% NP-40, 50 mM KCl, followed by the addition of nuclease-free water to achieve 20 μL. Samples were gently mixed, incubated at 0° C. for 1 h, and treated with SDS-free loading dye. Samples were loaded into a 4%-8% native polyacrylamide (PAGE) gel (29:1) containing TBE buffer (0.045 M tris, 0.045 M borate, and 0.01 M EDTA, pH 8.2-8.4), followed by separation at 100 V for 60 min at 5° C. The gels were visualized in a TBE buffer containing 1×SYBR Safe Stain and imaged using an Azure600 Imager. Band intensities were measured by NIH ImageJ, normalized in Excel, and plotted in GraphPad Prism.

### Example 1

### Design of Artificial Transcription Factors

Artificial transcription factors (ATFs) disclosed herein were designed by obtaining the hIFNG and/or TNF region of human chromosome 12. The target region is 400 base pairs (bp) upstream of the IFN-γ start codon. Analysis of this region with the ZFTools program identified fusion ZF proteins based on previously reported binding affinities to DNA triplets (Table 1). Sets of ZF proteins were selected to incorporate into ATFs, called ATF 1-4 (Tables 2A, 2B, and 3). The ATFs contain six-linked ZF proteins that recognize 18-bp regions throughout the hIFNG and/or TNF gene.

TABLE 1
Target nucleotide sequences in the
promoter region of the IFNG gene.

| | ATF 1 | ATF 2 | ATF 3 | ATF 4 |
|---|---|---|---|---|
| gene | CTTGTGAAA | GAAACTCTA | AGAATGGCA | ATGGTGTG |
| target | ATACGTAAT | ACTACAACA | CAGGTGGGC | AAGTAAAAGT |
| | (SEQ ID | (SEQ ID | (SEQ ID | (SEQ ID |
| | NO: 1) | NO: 2) | NO: 3) | NO: 4) |

TABLE 2A
Variable amino acids for IFNG gene recognition by ZF subunits.

| | ATF 1 | ATF 2 | ATF 3 | ATF 4 |
|---|---|---|---|---|
| ZF 1 | TTGNLTV | SPADLTR | DPGHLVR | HRTTLTN |
| | (SEQ ID NO: 5) | (SEQ ID NO: 11) | (SEQ ID NO: 17) | (SEQ ID NO: 23) |
| ZF 2 | SRRTCRA | SPADLTR | RSDELVR | QRANLRA |
| | (SEQ ID NO: 6) | (SEQ ID NO: 12) | (SEQ ID NO: 18) | (SEQ ID NO: 24) |
| ZF 3 | QKSSLIA | THLDLIR | RADNLTE | HRTTLTN |
| | (SEQ ID NO: 7) | (SEQ ID NO: 13) | (SEQ ID NO: 19) | (SEQ ID NO: 25) |
| ZF 4 | QRANLRA | QNSTLTE | QSGDLRR | QAGHLAS |
| | (SEQ ID NO: 8) | (SEQ ID NO: 14) | (SEQ ID NO: 20) | (SEQ ID NO: 26) |
| ZF 5 | RSDEL VR | THLDLIR | RRDELNV | RSDEL VR |
| | (SEQ ID NO: 9) | (SEQ ID NO: 15) | (SEQ ID NO: 21) | (SEQ ID NO: 27) |
| ZF 6 | TTGALTE | QSSNLVR | QLAHLRA | RRDELNV |
| | (SEQ ID NO: 10) | (SEQ ID NO: 16) | (SEQ ID NO: 22) | (SEQ ID NO: 28) |

TABLE 2B
Variable amino acids for TNF gene recognition by zinc finger subunits.

| | ATF 1 | ATF 2 | ATF 3 | ATF 4 |
|---|---|---|---|---|
| ZF 1 | ERSHLRE | HTGHLLE | RTDTLRD | QSGHLTE |
| | (SEQ ID NO: 43) | (SEQ ID NO: 49) | (SEQ ID NO: 55) | (SEQ ID NO: 61) |
| ZF 2 | SKKHLAE | SRGNLKS | RSDKLVR | HRTTLTN |
| | (SEQ ID NO: 44) | (SEQ ID NO: 50) | (SEQ ID NO: 56) | (SEQ ID NO: 62) |
| ZF 3 | SPADLTR | SKKALTE | DPGHLVR | QRAHLER |
| | (SEQ ID NO: 45) | (SEQ ID NO: 51) | (SEQ ID NO: 57) | (SEQ ID NO: 63) |
| ZF 4 | DPGHLVR | DCRDLAR | RRDELNV | ARGNLRT |
| | (SEQ ID NO: 46) | (SEQ ID NO: 52) | (SEQ ID NO: 58) | (SEQ ID NO: 64) |
| ZF 5 | TSGSLVR | RSDHLTN | RSDHLTN | DPGHLVR |
| | (SEQ ID NO: 47) | (SEQ ID NO: 53) | (SEQ ID NO: 59) | (SEQ ID NO: 65) |
| ZF 6 | TSGSLVR | APKALGW | RKDALRG | RADNLTE |
| | (SEQ ID NO: 48) | (SEQ ID NO: 54) | (SEQ ID NO: 60) | (SEQ ID NO: 66) |

TABLE 3
ATF protein scaffold showing the amino acid
sequences for six zinc finger (ZF) subunits
with variable regions for targeted
gene recognition.

| Component | Amino Acid Sequence (SEQ ID NO: 29) |
|---|---|
| ZF | LEPGEKPYKCPECGKSFSXXXXXXXHQRTHTGE |
| | KPYKCPECGKSFSXXXXXXXHQRTHTGEKPYKC |
| | PECGKSFSXXXXXXXHQRTHTGEKPYKCPECGK |
| | SFSSXXXXXXHQRTHTGEKPYKCPECGKSFSXX |
| | XXXXXHQRTHTGEKPYKCPECGKSFSXXXXXXX |
| | HQRTHTGKKTS |

¹ Underlined amino acids, shown as ‘XXXXXXX’, indicate the variable regions for targeted gene recognition.

ATFs 1-4 for targeting IFNG and TNF genes include three protein domains to evaluate transcription factor activity, each with a distinct function: fluorescence, gene targeting, and transcriptional activation (FIG. 1F; Tables 4-5). The fluorescent domain encodes Discosoma red (DsRed) for monitoring intracellular localization by fluorescence microscopy. The gene targeting domain comprises the zinc finger proteins that recognize the hIFNG gene.

The ATFs encode a transcriptional activation domain with three signals: a nuclear localization signal (NLS), four tandem copies of virus protein 16 (VP64), and a hemagglutinin A (HA) tag. NLS directs the ATF to the nucleus to access the intracellular genes. VP64 is a coactivator that facilitates transcription and translation. HA is included for protein tagging. Altogether, the sequences for ATFs 1-4 were codon optimized for expression in human cells (Tables 4A-4B), and incorporated into a mammalian expression vector (pTwist) with a CMV promoter. For biophysical studies, a sequence for recombinant expression in bacterial cells was also developed (FIG. 1G).

TABLE 4A
Amino acid sequences for mammalian expression of IFNG-targeting
ATF 1-4 and Aart6.

| | Amino Acid Sequence |
|---|---|
| ATF 1 | MASSEDVIKEFMRFKVRMEGSVNGHEFEIEGEGEGRPYEGTQTAKLKVTKGGPLPFAWDILSP |
| (SEQ ID | QFQYGSKVYVKHPADIPDYKKLSFPEGFKWERVMNFEDGGVVTVTQDSSLQDGSFIYKVKFIG |
| NO: 30) | VNFPSDGPVMQKKTMGWEASTERLYPRDGVLKGEIHKALKLKDGGHYLVEFKSIYMAKKPVQL |
| | PGYYYVDSKLDITSHNEDYTIVEQYERAEGRHHLFLGSGEGRGSLLTCGDVEENPGPLEPGEK |
| | PYKCPECGKSFSTTGNLTVHQRTHTGEKPYKCPECGKSFSSRRTCRAHQRTHTGEKPYKCPEC |
| | GKSFSQKSSLIAHQRTHTGEKPYKCPECGKSFSQRANLRAHQRTHTGEKPYKCPECGKSFSRS |
| | DELVRHQRTHTGEKPYKCPECGKSFSTTGALTEHQRTHTGKKTSPKKKRKVEASGSGRADAL |
| | DDFDLDMLGSDALDDFDLDMLGSDALDDFDLDMLGSDALDDFDLDMLYPYDVPDYA |
| ATF 2 | MASSEDVIKEFMRFKVRMEGSVNGHEFEIEGEGEGRPYEGTQTAKLKVTKGGPLPFAWDILSP |
| (SEQ ID | QFQYGSKVYVKHPADIPDYKKLSFPEGFKWERVMNFEDGGVVTVTQDSSLQDGSFIYKVKFIG |
| NO: 31) | VNFPSDGPVMQKKTMGWEASTERLYPRDGVLKGEIHKALKLKDGGHYLVEFKSIYMAKKPVQL |
| | PGYYYVDSKLDITSHNEDYTIVEQYERAEGRHHLFLGSGEGRGSLLTCGDVEENPGPLEPGEK |
| | PYKCPECGKSFSSPADLTRHQRTHTGEKPYKCPECGKSFSSPADLTRHQRTHTGEKPYKCPEC |
| | GKSFSTHLDLIRHQRTHTGEKPYKCPECGKSFSQNSTLTEHQRTHTGEKPYKCPECGKSFSTH |
| | LDLIRHQRTHTGEKPYKCPECGKSFSQSSNLVRHQRTHTGKKTSPKKKRKVEASGSGRADALD |
| | DFDLDMLGSDALDDFDLDMLGSDALDDFDLDMLGSDALDDFDLDMLYPYDVPDYA |
| ATF 3 | MASSEDVIKEFMRFKVRMEGSVNGHEFEIEGEGEGRPYEGTQTAKLKVTKGGPLPFAWDILSP |
| (SEQ ID | QFQYGSKVYVKHPADIPDYKKLSFPEGFKWERVMNFEDGGVVTVTQDSSLQDGSFIYKVKFIG |
| NO: 32) | VNFPSDGPVMQKKTMGWEASTERLYPRDGVLKGEIHKALKLKDGGHYLVEFKSIYMAKKPVQL |
| | PGYYYVDSKLDITSHNEDYTIVEQYERAEGRHHLFLGSGEGRGSLLTCGDVEENPGPLEPGEK |
| | PYKCPECGKSFSDPGHLVRHQRTHTGEKPYKCPECGKSFSRSDELVRHQRTHTGEKPYKCPEC |
| | GKSFSRADNLTEHQRTHTGEKPYKCPECGKSFSQSGDLRRHQRTHTGEKPYKCPECGKSFSRR |
| | DELNVHQRTHTGEKPYKCPECGKSFSQLAHLRAHQRTHTGKKTSPKKKRKVEASGSGRADALD |
| | DFDLDMLGSDALDDFDLDMLGSDALDDFDLDMLGSDALDDFDLDMLYPYDVPDYA |
| ATF 4 | MASSEDVIKEFMRFKVRMEGSVNGHEFEIEGEGEGRPYEGTQTAKLKVTKGGPLPFAWDILSP |
| (SEQ ID | QFQYGSKVYVKHPADIPDYKKLSFPEGFKWERVMNFEDGGVVTVTQDSSLQDGSFIYKVKFIG |
| NO: 33) | VNFPSDGPVMQKKTMGWEASTERLYPRDGVLKGEIHKALKLKDGGHYLVEFKSIYMAKKPVQL |
| | PGYYYVDSKLDITSHNEDYTIVEQYERAEGRHHLFLGSGEGRGSLLTCGDVEENPGPLEPGEK |
| | PYKCPECGKSFSDKKDLTRHQRTHTGEKPYKCPECGKSFSQSSSLVRHQRTHTGEKPYKCPEC |
| | GKSFSQRAHLERHQRTHTGEKPYKCPECGKSFSRKDNLKNHQRTHTGEKPYKCPECGKSFSQR |
| | ANLRAHQRTHTGEKPYKCPECGKSFSQKSSLIAHQRTHTGKKTSPKKKRKVEASGSGRADALD |
| | DFDLDMLGSDALDDFDLDMLGSDALDDFDLDMLGSDALDDFDLDMLYPYDVPDYA |
| Aart6 | MASSEDVIKEFMRFKVRMEGSVNGHEFEIEGEGEGRPYEGTQTAKLKVTKGGPLPFAWDILSP |
| (SEQ ID | QFQYGSKVYVKHPADIPDYKKLSFPEGFKWERVMNFEDGGVVTVTQDSSLQDGSFIYKVKFIG |
| NO: 34) | VNFPSDGPVMQKKTMGWEASTERLYPRDGVLKGEIHKALKLKDGGHYLVEFKSIYMAKKPVQL |
| | PGYYYVDSKLDITSHNEDYTIVEQYERAEGRHHLFLGSGEGRGSLLTCGDVEENPGPISEFGS |
| | SSSVAQAALEPGEKPYACPECGKSFSRSDHLAEHQRTHTGEKPYKCPECGKSFSDKKDLTRHQ |
| | RTHTGEKPYKCPECGKSFSQRANLRAHQRTHTGEKPYACPECGKSFSQLAHLRAHQRTHTGEK |
| | PYKCPECGKSFSREDLNHTHQRTHTGEKPYKCPECGKSFSRRDALNVHQRTHTGKKTSPKKKK |
| | KVEASGSGRADALDDFDLDMLGSDALDDFDLDMLGSDALDDFDLDMLGSDALDDFDLDMLYPY |
| | DVPDYA |

TABLE 4B
Amino acid sequences for mammalian expression of TNF-targeting ATF 1-4.

| | Amino Acid Sequence |
|---|---|
| ATF 1 | MASSEDVIKEFMRFKVRMEGSVNGHEFEIEGEGEGRPYEGTQTAKLKVTKGGPLPFAWDI |
| (SEQ ID | LSPQFQYGSKVYVKHPADIPDYKKLSFPEGFKWERVMNFEDGGVVTVTQDSSLQDGSFIYK |
| NO: 67) | VKFIGVNFPSDGPVMQKKTMGWEASTERLYPRDGVLKGEIHKALKLKDGGHYLVEFKSIY |
| | MAKKPVQLPGYYYVDSKLDITSHNEDYTIVEQYERAEGRHHLFLGSGEGRGSLLTCGDVE |
| | ENPGPLEPGEKPYKCPECGKSFSERSHLREHQRTHTGEKPYKCPECGKSFSSKKHLAEHQR |
| | THTGEKPYKCPECGKSFSSPADLTRHQRTHTGEKPYKCPECGKSFSDPGHLVRHQRTHTGE |
| | KPYKCPECGKSFSTSGSLVRHQRTHTGEKPYKCPECGKSFSTSGSLVRHQRTHTGKKTSPK |
| | KKRKVEASGSGRADALDDFDLDMLGSDALDDFDLDMLGSDALDDFDLDMLGSDALDDF |
| | DLDMLYPYDVPDYA |
| ATF 2 | MASSEDVIKEFMRFKVRMEGSVNGHEFEIEGEGEGRPYEGTQTAKLKVTKGGPLPFAWDIL |
| (SEQ ID | SPQFQYGSKVYVKHPADIPDYKKLSFPEGFKWERVMNFEDGGVVTVTQDSSLQDGSFIYK |
| NO: 68) | VKFIGVNFPSDGPVMQKKTMGWEASTERLYPRDGVLKGEIHKALKLKDGGHYLVEFKSIY |
| | MAKKPVQLPGYYYVDSKLDITSHNEDYTIVEQYERAEGRHHLFLGSGEGRGSLLTCGDVE |
| | ENPGPLEPGEKPYKCPECGKSFSHTGHLLEHQRTHTGEKPYKCPECGKSFSSRGNLKSHQR |
| | THTGEKPYKCPECGKSFSSKKALTEHQRTHTGEKPYKCPECGKSFSDCRDLARHQRTHTGE |
| | KPYKCPECGKSFSRSDHLTNHQRTHTGEKPYKCPECGKSFSAPKALGWHQRTHTGKKTSP |
| | KKKRKVEASGSGRADALDDFDLDMLGSDALDDFDLDMLGSDALDDFDLDMLGSDALDD |
| | FDLDMLYPYDVPDYA |
| ATF 3 | MASSEDVIKEFMRFKVRMEGSVNGHEFEIEGEGEGRPYEGTQTAKLKVTKGGPLPFAWDIL |
| (SEQ ID | SPQFQYGSKVYVKHPADIPDYKKLSFPEGFKWERVMNFEDGGVVTVTQDSSLQDGSFIYK |
| NO: 69) | VKFIGVNFPSDGPVMQKKTMGWEASTERLYPRDGVLKGEIHKALKLKDGGHYLVEFKSIY |
| | MAKKPVQLPGYYYVDSKLDITSHNEDYTIVEQYERAEGRHHLFLGSGEGRGSLLTCGDVE |
| | ENPGPLEPGEKPYKCPECGKSFSRTDTLRDHQRTHTGEKPYKCPECGKSFSRSDKLVRHQR |
| | THTGEKPYKCPECGKSFSDPGHLVRHQRTHTGEKPYKCPECGKSFSRRDELNVHQRTHTGE |
| | KPYKCPECGKSFSRSDHLTNHQRTHTGEKPYKCPECGKSFSRKDALRGHQRTHTGKKTSPK |
| | KKRKVEASGSGRADALDDFDLDMLGSDALDDFDLDMLGSDALDDFDLDMLGSDALDDF |
| | DLDMLYPYDVPDYA |
| ATF 4 | MASSEDVIKEFMRFKVRMEGSVNGHEFEIEGEGEGRPYEGTQTAKLKVTKGGPLPFAWDIL |
| (SEQ ID | SPQFQYGSKVYVKHPADIPDYKKLSFPEGFKWERVMNFEDGGVVTVTQDSSLQDGSFIYK |
| NO: 70) | VKFIGVNFPSDGPVMQKKTMGWEASTERLYPRDGVLKGEIHKALKLKDGGHYLVEFKSIY |
| | MAKKPVQLPGYYYVDSKLDITSHNEDYTIVEQYERAEGRHHLFLGSGEGRGSLLTCGDVE |
| | ENPGPLEPGEKPYKCPECGKSFSQSGHLTEHQRTHTGEKPYKCPECGKSFSHRTTLTNHQRT |
| | HTGEKPYKCPECGKSFSQRAHLERHQRTHTGEKPYKCPECGKSFSARGNLRTHQRTHTGE |
| | KPYKCPECGKSFSDPGHLVRHQRTHTGEKPYKCPECGKSFSRADNLTEHQRTHTGKKTSPK |
| | KKRKVEASGSGRADALDDFDLDMLGSDALDDFDLDMLGSDALDDFDLDMLGSDALDDF |
| | DLDMLYPYDVPDYA |

TABLE 4C
Nucleotide sequences for mammalian expression of IFNG-targeting ATF 1-4
and Aart6.

| | Nucleotide Sequence |
|---|---|
| ATF 1 | ATGGCCTCCTCCGAGGACGTCATCAAGGAGTTCATGCGCTTCAAGGTGCGCATGGAGGGC |
| (SEQ ID | TCCGTGAACGGCCACGAGTTCGAGATCGAGGGCGAGGGCGAGGGCCGCCCCTACGAGGG |
| NO: 71) | CACCCAGACCGCCAAGCTGAAGGTGACCAAGGGCGGCCCCCTGCCCTTCGCCTGGGACAT |
| | CCTGTCCCCCCAGTTCCAGTACGGCTCCAAGGTGTACGTGAAGCACCCCGCCGACATCCCC |
| | GACTACAAGAAGCTGTCCTTCCCCGAGGGCTTCAAGTGGGAGCGCGTGATGAACTTCGAG |
| | GACGGCGGCGTGGTGACCGTGACCCAGGACTCCTCCCTGCAGGACGGCTCCTTCATCTAC |
| | AAGGTGAAGTTCATCGGCGTGAACTTCCCCTCCGACGGCCCCGTAATGCAGAAGAAGACT |
| | ATGGGCTGGGAGGCCTCCACCGAGCGCCTGTACCCCCGCGACGGCGTGCTGAAGGGCGAG |
| | ATCCACAAGGCCCTGAAGCTGAAGGACGGCGGCCACTACCTGGTGGAGTTCAAGTCCATC |
| | TACATGGCCAAGAAGCCCGTGCAGCTGCCCGGCTACTACTACGTGGACTCCAAGCTGGAC |
| | ATCACCTCCCACAACGAGGACTACACCATCGTGGAGCAGTACGAGCGCGCCGAGGGCCGC |
| | CACCACCTGTTCCTGGGGTCAGGAGAGGGCAGAGGAAGTCTTCTAACATGCGGTGACGTG |
| | GAGGAGAATCCCGGCCCTCTGGAACCCGGGGAAAAGCCATACAAATGCCCAGAATGTGG |
| | AAAATCTTTCAGCACAACCGGTAACCTTACGGTACACCAGAGAACTCACACAGGTGAAAA |
| | ACCTTATAAATGTCCGGAGTGCGGCAAGTCATTTAGCTCACGCCGGACTTGCAGGGCACA |
| | TCAGCGCACTCACACTGGAGAGAAACCATACAAATGTCCCGAGTGTGGCAAGTCCTTCAG |
| | CCAAAAAAGTAGCCTGATAGCACACCAAAGAACCCACACAGGCGAGAAACCGTATAAGT |
| | GCCCTGAATGTGGCAAGAGTTTTTCCCAACGGGCCAATCTGCGGGCGCACCAACGCACAC |
| | ATACGGGTGAGAAGCCCTACAAGTGCCCCGAGTGCGGTAAGAGTTTTAGCAGGAGTGATG |
| | AGCTGGTGAGGCACCAAAGGACGCATACCGGTGAGAAGCCTTACAAGTGCCCAGAGTGTG |
| | GGAAATCATTCAGTACCACAGGGGCTCTTACGGAGCATCAAAGAACGCATACAGGCAAGA |
| | AAACTTCTCCCAAGAAGAAGAGGAAGGTGGAGGCCAGCGGTTCCGGACGGGCTGACGCAT |
| | TGGACGATTTTGATCTGGATATGCTGGGAAGTGACGCCCTCGATGATTTTGACCTTGACA |
| | TGCTTGGTTCGGATGCCCTTGATGACTTTGACCTCGACATGCTCGGCAGTGACGCCCTTG |
| | ATGATTTCGACCTGGACATGCTGTACCCCTACGATGTACCGGATTACGCTTGATAA |
| ATF 2 | ATGGCCTCCTCCGAGGACGTCATCAAGGAGTTCATGCGCTTCAAGGTGCGCATGGAGGGC |
| (SEQ ID | TCCGTGAACGGCCACGAGTTCGAGATCGAGGGCGAGGGCGAGGGCCGCCCCTACGAGGG |
| NO: 72) | CACCCAGACCGCCAAGCTGAAGGTGACCAAGGGCGGCCCCCTGCCCTTCGCCTGGGACAT |
| | CCTGTCCCCCCAGTTCCAGTACGGCTCCAAGGTGTACGTGAAGCACCCCGCCGACATCCCC |
| | GACTACAAGAAGCTGTCCTTCCCCGAGGGCTTCAAGTGGGAGCGCGTGATGAACTTCGAG |
| | GACGGCGGCGTGGTGACCGTGACCCAGGACTCCTCCCTGCAGGACGGCTCCTTCATCTAC |
| | AAGGTGAAGTTCATCGGCGTGAACTTCCCCTCCGACGGCCCCGTAATGCAGAAGAAGACT |
| | ATGGGCTGGGAGGCCTCCACCGAGCGCCTGTACCCCCGCGACGGCGTGCTGAAGGGCGAG |
| | ATCCACAAGGCCCTGAAGCTGAAGGACGGCGGCCACTACCTGGTGGAGTTCAAGTCCATC |
| | TACATGGCCAAGAAGCCCGTGCAGCTGCCCGGCTACTACTACGTGGACTCCAAGCTGGAC |
| | ATCACCTCCCACAACGAGGACTACACCATCGTGGAGCAGTACGAGCGCGCCGAGGGCCGC |
| | CACCACCTGTTCCTGGGGTCAGGAGAGGGCAGAGGAAGTCTTCTAACATGCGGTGACGTG |
| | GAGGAGAATCCCGGCCCTCTCGAACCCGGAGAAAAACCTTACAAGTGTCCTGAGTGTGGA |
| | AAGAGTTTCTCTAGCCCCGCTGATCTGACGCGCCACCAACGCACTCACACAGGGGAGAAA |
| | CCATACAAGTGCCCAGAATGTGGAAAAAGCTTCTCATCACCTGCCGACCTCACAAGACAT |
| | CAACGAACTCACACTGGAGAAAAACCATATAAGTGTCCCGAATGTGGTAAGAGCTTTTCT |
| | ACACATTTGGATTTGATACGGCATCAAAGGACGCATACAGGGGAGAAACCGTACAAATGC |
| | CCAGAGTGTGGCAAATCCTTTAGTCAAAACAGTACATTGACTGAGCATCAGCGGACTCAT |
| | ACGGGAGAAAAGCCCTACAAGTGTCCGGAGTGTGGAAAGTCTTTTTCCACCCATCTGGAC |
| | CTCATTCGACACCAACGGACACATACCGGCGAAAAGCCTTATAAGTGCCCCGAATGCGGA |
| | AAAAGCTTCTCTCAATCTAGTAATCTTGTGCGCCATCAGAGGACACACACAGGTAAAAAA |
| | ACGTCTCCCAAGAAGAAGAGGAAGGTGGAGGCCAGCGGTTCCGGACGGGCTGACGCATT |
| | GGACGATTTTGATCTGGATATGCTGGGAAGTGACGCCCTCGATGATTTTGACCTTGACATG |
| | CTTGGTTCGGATGCCCTTGATGACTTTGACCTCGACATGCTCGGCAGTGACGCCCTTGATG |
| | ATTTCGACCTGGACATGCTGTACCCCTACGATGTACCGGATTACGCTTGATAA |
| ATF 3 | ATGGCCTCCTCCGAGGACGTCATCAAGGAGTTCATGCGCTTCAAGGTGCGCATGGAGGGC |
| (SEQ ID | TCCGTGAACGGCCACGAGTTCGAGATCGAGGGCGAGGGCGAGGGCCGCCCCTACGAGGG |
| NO: 73) | CACCCAGACCGCCAAGCTGAAGGTGACCAAGGGCGGCCCCCTGCCCTTCGCCTGGGACAT |
| | CCTGTCCCCCCAGTTCCAGTACGGCTCCAAGGTGTACGTGAAGCACCCCGCCGACATCCCC |
| | GACTACAAGAAGCTGTCCTTCCCCGAGGGCTTCAAGTGGGAGCGCGTGATGAACTTCGAG |
| | GACGGCGGCGTGGTGACCGTGACCCAGGACTCCTCCCTGCAGGACGGCTCCTTCATCTAC |
| | AAGGTGAAGTTCATCGGCGTGAACTTCCCCTCCGACGGCCCCGTAATGCAGAAGAAGACT |
| | ATGGGCTGGGAGGCCTCCACCGAGCGCCTGTACCCCCGCGACGGCGTGCTGAAGGGCGAG |
| | ATCCACAAGGCCCTGAAGCTGAAGGACGGCGGCCACTACCTGGTGGAGTTCAAGTCCATC |
| | TACATGGCCAAGAAGCCCGTGCAGCTGCCCGGCTACTACTACGTGGACTCCAAGCTGGAC |
| | ATCACCTCCCACAACGAGGACTACACCATCGTGGAGCAGTACGAGCGCGCCGAGGGCCGC |
| | CACCACCTGTTCCTGGGGTCAGGAGAGGGCAGAGGAAGTCTTCTAACATGCGGTGACGTG |
| | GAGGAGAATCCCGGCCCTCTGGAGCCTGGGGAAAAACCCTATAAGTGTCCAGAATGCGGA |
| | AAGAGTTTTAGCGATCCAGGCCATTTGGTGCGCCACCAGCGCACTCACACGGGCGAGAAA |
| | CCCTATAAATGCCCGGAATGTGGAAAATCATTCAGCAGATCCGATGAGCTTGTCCGGCAC |
| | CAGAGGACGCACACCGGTGAGAAGCCCTACAAATGCCCGGAATGCGGAAAGTCATTCAG |
| | CCGCGCGGACAATCTTACTGAACATCAGAGGACTCACACGGGTGAGAAACCGTACAAGTG |
| | CCCCGAATGCGGGAAAAGTTTCTCTCAGTCTGGTGACCTGCGGCGGCACCAGCGAACACA |
| | TACGGGGGAGAAACCATATAAATGCCCAGAGTGCGGCAAAAGCTTTAGCCGACGGGACG |
| | AGCTGAATGTACATCAGCGGACCCACACAGGTGAAAAGCCATATAAGTGCCCTGAGTGCG |
| | GCAAGTCCTTTTCTCAGTTGGCACACTTGCGGGCACATCAACGGACACACACGGGTAAAA |
| | AAACAAGCCCCAAGAAGAAGAGGAAGGTGGAGGCCAGCGGTTCCGGACGGGCTGACGCA |
| | TTGGACGATTTTGATCTGGATATGCTGGGAAGTGACGCCCTCGATGATTTTGACCTTGACA |
| | TGCTTGGTTCGGATGCCCTTGATGACTTTGACCTCGACATGCTCGGCAGTGACGCCCTTGA |
| | TGATTTCGACCTGGACATGCTGTACCCCTACGATGTACCGGATTACGCTTGATAA |
| ATF 4 | ATGGCCTCCTCCGAGGACGTCATCAAGGAGTTCATGCGCTTCAAGGTGCGCATGGAGGGC |
| (SEQ ID | TCCGTGAACGGCCACGAGTTCGAGATCGAGGGCGAGGGCGAGGGCCGCCCCTACGAGGG |
| NO: 74) | CACCCAGACCGCCAAGCTGAAGGTGACCAAGGGCGGCCCCCTGCCCTTCGCCTGGGACAT |
| | CCTGTCCCCCCAGTTCCAGTACGGCTCCAAGGTGTACGTGAAGCACCCCGCCGACATCCCC |
| | GACTACAAGAAGCTGTCCTTCCCCGAGGGCTTCAAGTGGGAGCGCGTGATGAACTTCGAG |
| | GACGGCGGCGTGGTGACCGTGACCCAGGACTCCTCCCTGCAGGACGGCTCCTTCATCTAC |
| | AAGGTGAAGTTCATCGGCGTGAACTTCCCCTCCGACGGCCCCGTAATGCAGAAGAAGACT |
| | ATGGGCTGGGAGGCCTCCACCGAGCGCCTGTACCCCCGCGACGGCGTGCTGAAGGGCGAG |
| | ATCCACAAGGCCCTGAAGCTGAAGGACGGCGGCCACTACCTGGTGGAGTTCAAGTCCATC |
| | TACATGGCCAAGAAGCCCGTGCAGCTGCCCGGCTACTACTACGTGGACTCCAAGCTGGAC |
| | ATCACCTCCCACAACGAGGACTACACCATCGTGGAGCAGTACGAGCGCGCCGAGGGCCGC |
| | CACCACCTGTTCCTGGGGTCAGGAGAGGGCAGAGGAAGTCTTCTAACATGCGGTGACGTG |
| | GAGGAGAATCCCGGCCCTCTGGAGCCAGGAGAGAAGCCTTATAAGTGCCCAGAATGTGGA |
| | AAATCTTTTTCTGACAAGAAAGATCTGACACGCCACCAGAGAACACACACGGGAGAAAAG |
| | CCGTACAAATGTCCCGAATGTGGCAAATCATTCTCACAATCCTCTTCCCTTGTAAGACACC |
| | AAAGGACACACACTGGCGAAAAGCCTTATAAGTGCCCCGAGTGTGGGAAAAGCTTCAGTC |
| | AACGAGCCCACCTGGAGCGGCACCAGAGAACACATACAGGGGAAAAACCGTATAAGTGC |
| | CCGGAGTGTGGTAAGAGTTTTTCCCGGAAAGACAATCTCAAGAATCATCAGCGAACGCAT |
| | ACTGGAGAAAAGCCTTACAAGTGTCCAGAGTGTGGCAAATCTTTCAGCCAGAGGGCCAAC |
| | CTCAGAGCACATCAACGCACACACACGGGGGAGAAACCGTATAAATGCCCAGAATGCGG |
| | TAAAAGCTTTTCTCAGAAGAGTAGTCTTATTGCGCATCAACGGACACACACGGGAAAGAA |
| | AACCAGTCCCAAGAAGAAGAGGAAGGTGGAGGCCAGCGGTTCCGGACGGGCTGACGCAT |
| | TGGACGATTTTGATCTGGATATGCTGGGAAGTGACGCCCTCGATGATTTTGACCTTGACAT |
| | GCTTGGTTCGGATGCCCTTGATGACTTTGACCTCGACATGCTCGGCAGTGACGCCCTTGAT |
| | GATTTCGACCTGGACATGCTGTACCCCTACGATGTACCGGATTACGCTTGATAA |
| Aart6 | ATGGCCTCCTCCGAGGACGTCATCAAGGAGTTCATGCGCTTCAAGGTGCGCATGGAGGGC |
| (SEQ | TCCGTGAACGGCCACGAGTTCGAGATCGAGGGCGAGGGCGAGGGCCGCCCCTACGAGGG |
| ID NO: | CACCCAGACCGCCAAGCTGAAGGTGACCAAGGGCGGCCCCCTGCCCTTCGCCTGGGACAT |
| 75) | CCTGTCCCCCCAGTTCCAGTACGGCTCCAAGGTGTACGTGAAGCACCCCGCCGACATCCCC |
| | GACTACAAGAAGCTGTCCTTCCCCGAGGGCTTCAAGTGGGAGCGCGTGATGAACTTCGAG |
| | GACGGCGGCGTGGTGACCGTGACCCAGGACTCCTCCCTGCAGGACGGCTCCTTCATCTAC |
| | AAGGTGAAGTTCATCGGCGTGAACTTCCCCTCCGACGGCCCCGTAATGCAGAAGAAGACT |
| | ATGGGCTGGGAGGCCTCCACCGAGCGCCTGTACCCCCGCGACGGCGTGCTGAAGGGCGAG |
| | ATCCACAAGGCCCTGAAGCTGAAGGACGGCGGCCACTACCTGGTGGAGTTCAAGTCCATC |
| | TACATGGCCAAGAAGCCCGTGCAGCTGCCCGGCTACTACTACGTGGACTCCAAGCTGGAC |
| | ATCACCTCCCACAACGAGGACTACACCATCGTGGAGCAGTACGAGCGCGCCGAGGGCCGC |
| | CACCACCTGTTCCTGGGGTCAGGAGAGGGCAGAGGAAGTCTTCTAACATGCGGTGACGTG |
| | GAGGAGAATCCCGGCCCTATATCCGAATTTGGTAGCTCATCTAGTGTCGCGCAAGCGGCTT |
| | TGGAGCCGGGTGAGAAACCCTACGCATGTCCGGAATGCGGCAAATCCTTTTCCCGGTCTG |
| | ATCACCTGGCGGAGCACCAAAGGACTCACACGGGTGAAAAACCGTATAAATGTCCAGAGT |
| | GTGGTAAAAGCTTTAGCGATAAAAAAGATCTTACAAGGCACCAGCGGACGCATACGGGCG |
| | AGAAACCTTACAAATGCCCCGAGTGCGGCAAATCATTTTCCCAAAGAGCTAACCTGCGAG |
| | CACACCAAAGAACTCACACTGGAGAGAAGCCATATGCCTGTCCCGAATGCGGGAAGAGTT |
| | TCTCTCAGTTGGCCCACCTTAGAGCTCACCAGCGAACGCATACTGGCGAAAAGCCATACA |
| | AGTGTCCGGAATGTGGTAAAAGTTTCAGCCGCGAAGATCTGAATCACACTCACCAGAGGA |
| | CTCATACCGGCGAGAAACCGTACAAGTGTCCGGAGTGCGGTAAGTCCTTTTCAAGAAGAG |
| | ACGCGTTGAACGTCCACCAGCGCACCCACACTGGTAAGAAGACGAGTCCCAAGAAGAAG |
| | AAGAAGGTGGAGGCCAGCGGTTCCGGACGGGCTGACGCATTGGACGATTTTGATCTGGAT |
| | ATGCTGGGAAGTGACGCCCTCGATGATTTTGACCTTGACATGCTTGGTTCGGATGCCCTTG |
| | ATGACTTTGACCTCGACATGCTCGGCAGTGACGCCCTTGATGATTTCGACCTGGACATGCT |
| | GTACCCCTACGATGTACCGGATTACGCTTGATAA |

TABLE 4D
Nucleotide sequences for mammalian expression of TNF-targeting ATF 1-4.

| | Nucleotide Sequence |
|---|---|
| ATF 1 | ATGGCCTCCTCCGAGGACGTCATCAAGGAGTTCATGCGCTTCAAGGTGCGCATGGAGG |
| (SEQ | GCTCCGTGAACGGCCACGAGTTCGAGATCGAAGGAGAAGGTGAAGGAAGACCCTACG |
| ID NO: | AGGGCACCCAGACCGCCAAGCTGAAGGTGACCAAGGGCGGCCCCCTGCCCTTCGCCTG |
| 76) | GGACATCCTGTCCCCCCAGTTCCAGTACGGCTCCAAGGTGTACGTGAAGCACCCCGCCG |
| | ACATCCCCGACTACAAGAAGCTGTCCTTCCCCGAGGGCTTCAAGTGGGAGCGCGTGAT |
| | GAACTTCGAGGACGGCGGCGTGGTGACCGTGACCCAGGACTCCTCCCTGCAGGACGGC |
| | TCCTTCATCTACAAGGTGAAGTTCATCGGCGTGAACTTCCCCTCCGACGGCCCCGTAAT |
| | GCAGAAGAAGACTATGGGCTGGGAGGCCTCCACCGAGCGCCTGTACCCCCGCGACGGC |
| | GTGCTGAAGGGCGAGATCCACAAGGCCCTGAAGCTGAAGGACGGCGGCCACTACCTG |
| | GTGGAGTTCAAGTCCATCTACATGGCCAAGAAGCCCGTGCAGCTGCCCGGCTACTACTA |
| | CGTGGACTCCAAGCTGGACATCACCTCCCACAACGAGGACTACACCATCGTGGAGCAG |
| | TACGAGCGCGCCGAGGGCCGCCACCACCTGTTCCTGGGGTCAGGAGAGGGCAGAGGA |
| | AGTCTTCTAACATGCGGTGACGTGGAGGAGAATCCCGGCCCTCTCGAGCCAGGCGAGA |
| | AACCTTATAAATGTCCCGAATGTGGCAAATCCTTCAGTGAGAGAAGTCACCTTCGGGAA |
| | CACCAGAGAACCCACACAGGGGAAAAACCTTATAAGTGCCCTGAATGCGGCAAATCCT |
| | TCTCCAGTAAAAAACACCTCGCGGAACATCAAAGAACCCACACAGGTGAGAAGCCATA |
| | TAAGTGCCCGGAATGTGGTAAGTCCTTTTCCAGTCCGGCTGATTTGACTAGGCATCAGC |
| | GCACGCATACGGGTGAGAAGCCGTATAAATGCCCGGAATGCGGCAAGTCTTTCAGTGAT |
| | CCGGGCCACCTGGTCCGACATCAACGGACTCATACTGGAGAAAAACCATACAAGTGCC |
| | CGGAATGCGGAAAGAGCTTTAGTACATCCGGAAGTCTCGTCAGACACCAACGCACTCA |
| | TACGGGCGAAAAGCCTTACAAATGCCCCGAGTGTGGTAAGTCCTTCAGCACGAGTGGG |
| | AGTCTTGTGCGGCACCAGCGGACACACACAGGAAAGAAAACGAGTCCCAAGAAGAAG |
| | AGGAAGGTGGAGGCCAGCGGTTCCGGACGGGCTGACGCATTGGACGATTTTGATCTGG |
| | ATATGCTGGGAAGTGACGCCCTCGATGATTTTGACCTTGACATGCTTGGTTCGGATGCCC |
| | TTGATGACTTTGACCTCGACATGCTCGGCAGTGACGCCCTTGATGATTTCGACCTGGAC |
| | ATGCTGTACCCCTACGATGTACCGGATTACGCTTGATAA |
| ATF 2 | ATGGCCTCCTCCGAGGACGTCATCAAGGAGTTCATGCGCTTCAAGGTGCGCATGGAGG |
| (SEQ | GCTCCGTGAACGGCCACGAGTTCGAGATCGAAGGAGAAGGTGAAGGGCGCCCCTACG |
| ID NO: | AGGGCACCCAGACCGCCAAGCTGAAGGTGACCAAGGGCGGCCCCCTGCCCTTCGCCTG |
| 77) | GGACATCCTGTCCCCCCAGTTCCAGTACGGCTCCAAGGTGTACGTGAAGCACCCCGCCG |
| | ACATCCCCGACTACAAGAAGCTGTCCTTCCCCGAGGGCTTCAAGTGGGAGCGCGTGAT |
| | GAACTTCGAGGACGGCGGCGTGGTGACCGTGACCCAGGACTCCTCCCTGCAGGACGGC |
| | TCCTTCATCTACAAGGTGAAGTTCATCGGCGTGAACTTCCCCTCCGACGGCCCCGTAAT |
| | GCAGAAGAAGACTATGGGCTGGGAGGCCTCCACCGAGCGCCTGTACCCCCGCGACGGC |
| | GTGCTGAAGGGCGAGATCCACAAGGCCCTGAAGCTGAAGGACGGCGGCCACTACCTG |
| | GTGGAGTTCAAGTCCATCTACATGGCCAAGAAGCCCGTGCAGCTGCCCGGCTACTACTA |
| | CGTGGACTCCAAGCTGGACATCACCTCCCACAACGAGGACTACACCATCGTGGAGCAG |
| | TACGAGCGCGCCGAGGGCCGCCACCACCTGTTCCTGGGGTCAGGAGAGGGCAGAGGA |
| | AGTCTTCTAACATGCGGTGACGTGGAGGAGAATCCCGGCCCTTTGGAGCCGGGTGAGA |
| | AGCCTTATAAATGTCCTGAGTGCGGGAAAAGTTTCAGCCATACTGGACACCTTCTCGAA |
| | CACCAGAGGACGCACACCGGGGAGAAGCCATATAAATGCCCCGAGTGTGGTAAGAGTT |
| | TTAGTTCACGCGGCAACCTCAAAAGCCATCAAAGGACTCATACAGGAGAGAAGCCATA |
| | CAAATGTCCTGAATGTGGCAAAAGCTTCTCTTCAAAGAAGGCGCTTACTGAGCACCAG |
| | CGCACTCACACTGGCGAAAAGCCGTACAAATGTCCGGAATGCGGGAAGAGTTTTAGTG |
| | ATTGTCGGGACTTGGCAAGGCACCAGCGCACGCATACCGGGGAAAAACCATACAAATG |
| | CCCGGAGTGCGGGAAGTCCTTTTCACGATCTGATCATCTGACAAACCACCAGCGGACG |
| | CACACGGGGGAGAAGCCTTACAAATGCCCCGAATGCGGCAAATCCTTTTCTGCACCGA |
| | AGGCTCTTGGGTGGCATCAGCGAACACATACCGGAAAAAAGACAAGTCCCAAGAAGA |
| | AGAGGAAGGTGGAGGCCAGCGGTTCCGGACGGGCTGACGCATTGGACGATTTTGATCT |
| | GGATATGCTGGGAAGTGACGCCCTCGATGATTTTGACCTTGACATGCTTGGTTCGGATGC |
| | CCTTGATGACTTTGACCTCGACATGCTCGGCAGTGACGCCCTTGATGATTTCGACCTGG |
| | ACATGCTGTACCCCTACGATGTACCGGATTACGCTTGATAA |
| ATF 3 | ATGGCCTCCTCCGAGGACGTCATCAAGGAGTTCATGCGCTTCAAGGTGCGCATGGAGG |
| (SEQ | GCTCCGTGAACGGCCACGAGTTCGAGATCGAAGGAGAAGGAGAAGGTAGACCCTACGA |
| ID NO: | GGGCACCCAGACCGCCAAGCTGAAGGTGACCAAGGGCGGCCCCCTGCCCTTCGCCTGG |
| 78) | GACATCCTGTCCCCCCAGTTCCAGTACGGCTCCAAGGTGTACGTGAAGCACCCCGCCGA |
| | CATCCCCGACTACAAGAAGCTGTCCTTCCCCGAGGGCTTCAAGTGGGAGCGCGTGATG |
| | AACTTCGAGGACGGCGGCGTGGTGACCGTGACCCAGGACTCCTCCCTGCAGGACGGCT |
| | CCTTCATCTACAAGGTGAAGTTCATCGGCGTGAACTTCCCCTCCGACGGCCCCGTAATG |
| | CAGAAGAAGACTATGGGCTGGGAGGCCTCCACCGAGCGCCTGTACCCCCGCGACGGCG |
| | TGCTGAAGGGCGAGATCCACAAGGCCCTGAAGCTGAAGGACGGCGGCCACTACCTGGT |
| | GGAGTTCAAGTCCATCTACATGGCCAAGAAGCCCGTGCAGCTGCCCGGCTACTACTACG |
| | TGGACTCCAAGCTGGACATCACCTCCCACAACGAGGACTACACCATCGTGGAGCAGTA |
| | CGAGCGCGCCGAGGGCCGCCACCACCTGTTCCTGGGGTCAGGAGAGGGCAGAGGAAG |
| | TCTTCTAACATGCGGTGACGTGGAGGAGAATCCCGGCCCTCTTGAACCCGGGGAGAAA |
| | CCGTATAAGTGTCCAGAGTGCGGCAAGAGCTTTTCCCGCACAGACACACTTCGGGACC |
| | ATCAGCGGACACATACGGGAGAGAAGCCTTATAAATGCCCCGAATGCGGAAAATCTTTC |
| | TCCAGGAGTGATAAACTGGTTCGGCACCAACGCACGCATACAGGTGAAAAGCCTTACA |
| | AATGCCCCGAGTGTGGTAAGTCCTTCTCAGATCCAGGGCACCTGGTGAGGCACCAACG |
| | GACTCACACCGGCGAGAAACCATACAAGTGCCCCGAGTGCGGCAAAAGCTTCAGCAGG |
| | CGCGATGAACTCAATGTTCACCAGAGGACACACACTGGAGAGAAACCGTACAAGTGTC |
| | CGGAATGCGGCAAGAGTTTTTCTCGGTCTGATCATTTGACCAACCACCAACGAACGCAC |
| | ACTGGCGAAAAGCCATATAAATGTCCCGAGTGCGGAAAGTCTTTCTCTCGGAAGGACGC |
| | TCTGAGAGGTCATCAACGGACTCACACAGGCAAAAAGACTAGTCCCAAGAAGAAGAGG |
| | AAGGTGGAGGCCAGCGGTTCCGGACGGGCTGACGCATTGGACGATTTTGATCTGGATA |
| | TGCTGGGAAGTGACGCCCTCGATGATTTTGACCTTGACATGCTTGGTTCGGATGCCCTTG |
| | ATGACTTTGACCTCGACATGCTCGGCAGTGACGCCCTTGATGATTTCGACCTGGACATG |
| | CTGTACCCCTACGATGTACCGGATTACGCTTGATAA |
| ATF 4 | ATGGCCTCCTCCGAGGACGTCATCAAGGAGTTCATGCGCTTCAAGGTGCGCATGGAGGG |
| (SEQ | CTCCGTGAACGGCCACGAGTTCGAGATCGAAGGAGAAGGAGAAGGAAGACCCTACGA |
| ID NO: | GGGCACCCAGACCGCCAAGCTGAAGGTGACCAAGGGCGGCCCCCTGCCCTTCGCCTGG |
| 79) | GACATCCTGTCCCCCCAGTTCCAGTACGGCTCCAAGGTGTACGTGAAGCACCCCGCCGA |
| | CATCCCCGACTACAAGAAGCTGTCCTTCCCCGAGGGCTTCAAGTGGGAGCGCGTGATG |
| | AACTTCGAGGACGGCGGCGTGGTGACCGTGACCCAGGACTCCTCCCTGCAGGACGGCT |
| | CCTTCATCTACAAGGTGAAGTTCATCGGCGTGAACTTCCCCTCCGACGGCCCCGTAATG |
| | CAGAAGAAGACTATGGGCTGGGAGGCCTCCACCGAGCGCCTGTACCCCCGCGACGGCG |
| | TGCTGAAGGGCGAGATCCACAAGGCCCTGAAGCTGAAGGACGGCGGCCACTACCTGGT |
| | GGAGTTCAAGTCCATCTACATGGCCAAGAAGCCCGTGCAGCTGCCCGGCTACTACTACG |
| | TGGACTCCAAGCTGGACATCACCTCCCACAACGAGGACTACACCATCGTGGAGCAGTA |
| | CGAGCGCGCCGAGGGCCGCCACCACCTGTTCCTGGGGTCAGGAGAGGGCAGAGGAAG |
| | TCTTCTAACATGCGGTGACGTGGAGGAGAATCCCGGCCCTCTCGAGCCGGGTGAAAAA |
| | CCGTACAAGTGTCCCGAGTGTGGCAAGTCTTTCTCTCAGTCAGGTCATTTGACGGAACA |
| | TCAGAGAACACATACAGGAGAAAAACCCTATAAGTGTCCGGAATGTGGAAAATCCTTTT |
| | CACATAGGACGACGTTGACCAATCATCAACGGACTCACACCGGCGAGAAACCCTACAA |
| | GTGTCCAGAATGTGGGAAGTCTTTTTCTCAACGGGCGCACCTGGAAAGACACCAGCGG |
| | ACGCACACTGGCGAAAAACCCTATAAATGTCCTGAATGCGGGAAGAGTTTCTCAGCTAG |
| | GGGAAACCTCCGGACTCACCAGAGAACCCACACGGGAGAAAAGCCATATAAGTGTCCT |
| | GAGTGCGGAAAATCATTCTCCGACCCTGGACACCTTGTTCGCCATCAGCGCACGCACAC |
| | AGGAGAGAAACCGTATAAATGCCCCGAATGCGGCAAATCTTTCAGCCGAGCGGACAATC |
| | TCACTGAGCACCAACGCACACACACTGGTAAGAAAACGAGCCCCAAGAAGAAGAGGA |
| | AGGTGGAGGCCAGCGGTTCCGGACGGGCTGACGCATTGGACGATTTTGATCTGGATATG |
| | CTGGGAAGTGACGCCCTCGATGATTTTGACCTTGACATGCTTGGTTCGGATGCCCTTGAT |
| | GACTTTGACCTCGACATGCTCGGCAGTGACGCCCTTGATGATTTCGACCTGGACATGCT |
| | GTACCCCTACGATGTACCGGATTACGCTTGATAA |

TABLE 5
Amino acid sequences for the non-zinc finger ATF subcomponents.

| Component | Amino Acid Sequence |
|---|---|
| DsRed | MASSEDVIKEFMRFKVRMEGSVNGHEFEIEGEGEGRPYEGTQTAKLKVTKGGPLPFAW |
| (SEQ ID NO: | DILSPQFQYGSKVYVKHPADIPDYKKLSFPEGFKWERVMNFEDGGVVTVTQDSSLQDG |
| 35) | SFIYKVKFIGVNFPSDGPVMQKKTMGWEASTERLYPRDGVLKGEIHKALKLKDGGHYL |
| | VEFKSIYMAKKPVQLPGYYYVDSKLDITSHNEDYTIVEQYERAEGRHHLFL |
| GSG linker | GSG |
| (SEQ ID NO: | |
| 36) | |
| T2A | EGRGSLLTCGDVEENPGP |
| (SEQ ID NO: | |
| 37) | |
| NLS | PKKKRKV |
| (SEQ ID NO: | |
| 38) | |
| VP64 | DALDDFDLDMLGSDALDDFDLDMLGSDALDDFDLDMLGSDALDDFDLDML |
| (SEQ ID NO: | |
| 39) | |
| HA tag | YPYDVPDYA |
| (SEQ ID NO: | |
| 40) | |

### Example 2

### Evaluating ATF Activity in Human Jurkat T Cells

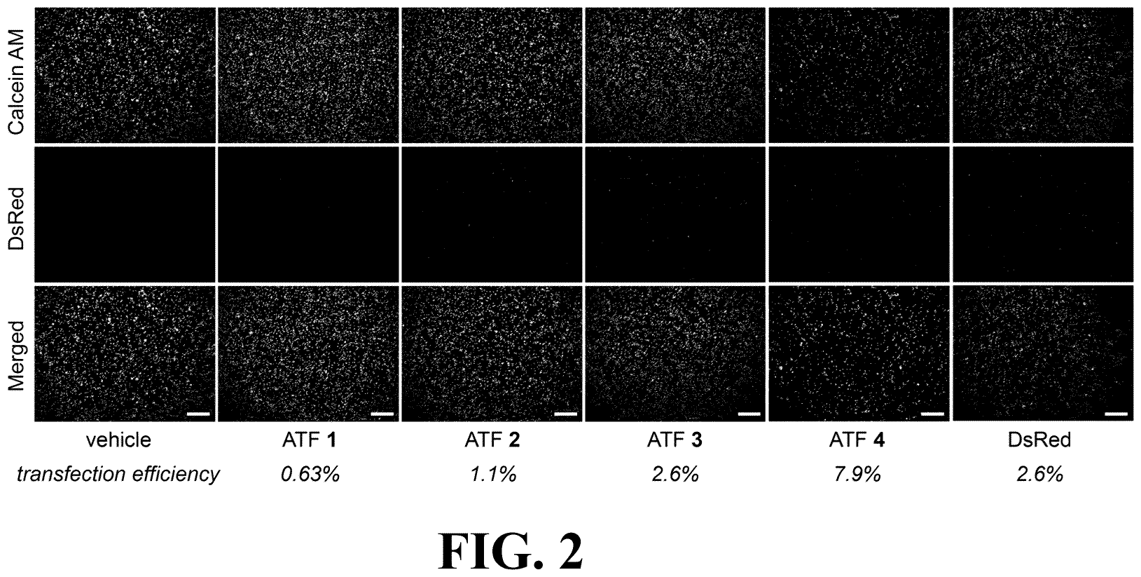

The biological activity of the mammalian ATFs for targeting hIFNG and hTNF genes was evaluated in human Jurkat T cells. Although plasmid delivery into T cells is notoriously challenging, successful transfection was achieved using electroporation. FIG. 2 shows fluorescent images of the Jurkat cells, which indicate the transfection efficiency (DsRed) and cell viability (Calcein AM). Transfection efficiency was determined by comparing the red fluorescent cells with the total viable cells. The transfection was successful for all plasmids but varied slightly for each ATF. Upon confirming that the cells produce the DsRed protein, each ATF was evaluated for its effects on IFN-γ and TNF expression.

### Example 3

### ATF-Mediated IFN-γ Production

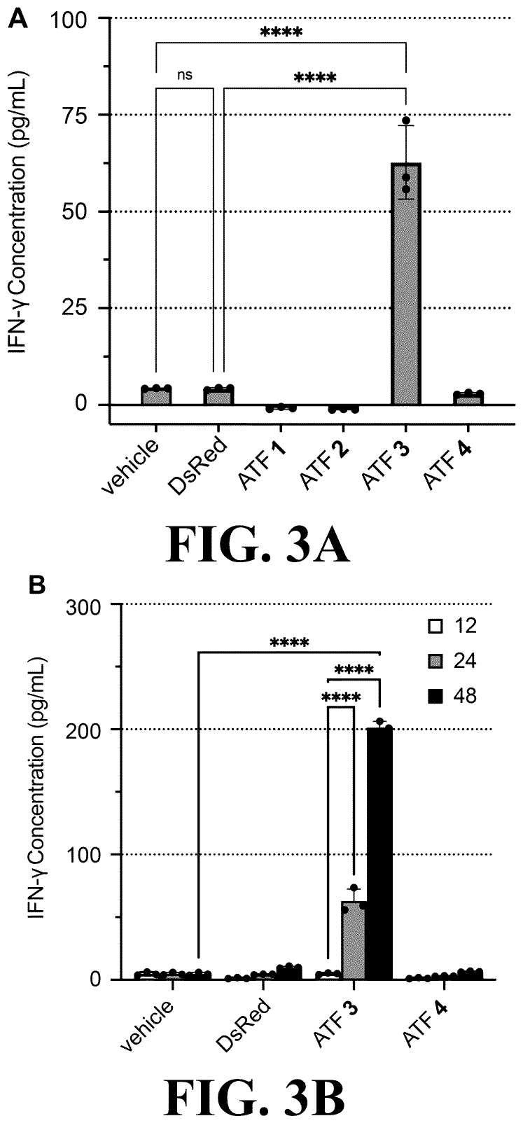

Human Jurkat T cells produce minimal quantities of IFN-γ under steady-state conditions. Although human patients typically exhibit IFN-γ concentrations of 1 pg/mL, treatment with PMA and ionomycin typically provides potent T cell activation by upregulating protein kinase C and facilitating the transfer of Ca²⁺ ions, respectively. The activity of each ATF was established in human Jurkat T cells through the measurement of IFN-γ expression using enzyme-linked immunosorbent assays (ELISAs). ATF 1-4 constructs were evaluated for their ability to mediate IFN-γ expression compared to the vehicle (buffer only) and DsRed plasmid (FIG. 3A). The treatment with ATFs 1, 2, and 4 showed low levels of IFN-γ, indicating that these ATF constructs do not activate IFN-γ transcription and translation. Intriguingly, ATFs 1 and 2 appear to decrease IFN-γ expression, which may reflect the inhibition of the hIFNG gene. Treatment with ATF 3 increased IFN-γ levels to 62.7 pg/mL after a 24 h incubation, which is about a 14-fold increase compared to baseline levels. Increased IFN-γ expression by ATF 3 is also sustained over time (up to 48 h), which indicates that transcriptional activation is not an artifact from the transfection conditions (FIG. 3B).

Two negative controls were tested to establish baseline IFN-γ production levels: (1) electroporated cells in the absence of plasmid, which is termed “vehicle”; (2) an empty plasmid that encodes DsRed without an ATF. Treatment of Jurkat cells with the “vehicle” and DsRed generated only 4.2-4.3 pg/mL of IFN-γ, indicating that neither condition significantly increases IFN-γ expression.

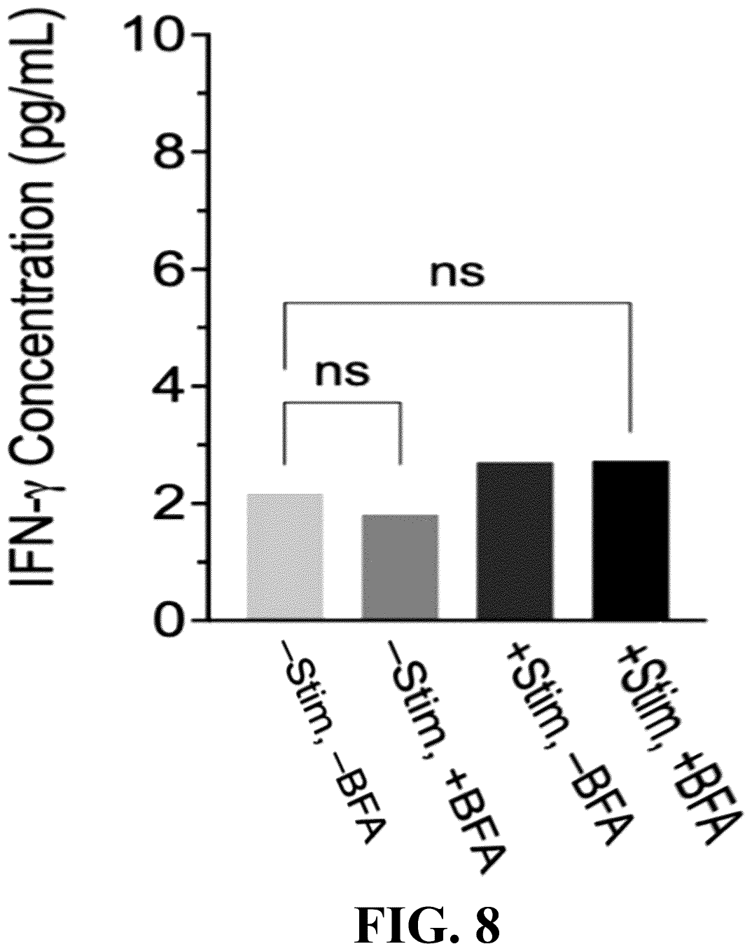

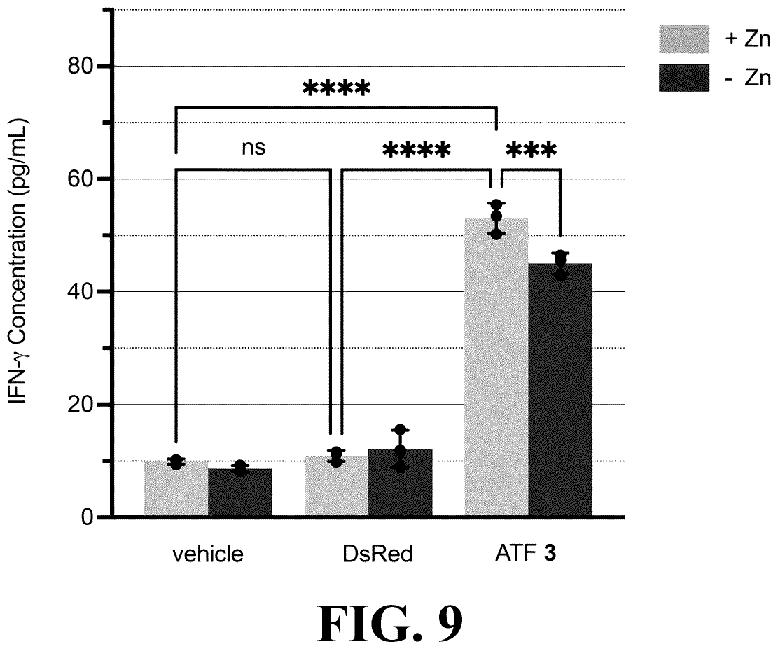

Additional testing of ATF activity further determined the baseline levels of IFN-γ production in Jurkat cells (FIG. 8). Incubating Jurkat cells with PMA/ionomycin showed no increase in IFN-γ production, indicating that PMA/ionomycin treatment alone does not increase IFN-γ expression. Brefeldin A (BFA) inhibits vesicle formation and halts protein export, allowing for accurate measurement of IFN-γ from the cell lysate. Incubating the cells with BFA alone also showed no increase in IFN-γ production. The folding of ZF proteins in the ββα-fold conformation is stabilized by zinc ions. To assess the role of zinc ions for ATF activity (FIG. 9), ATF-transfected Jurkat cells were incubated with ZnCl₂ (10 μM) for 24 h. These groups showed marginally higher IFN-γ expression than cells without ZnCl₂, demonstrating that zinc ions play a role in supporting ATF activity.

### Example 4

### Activation of Interferon-Stimulated Genes

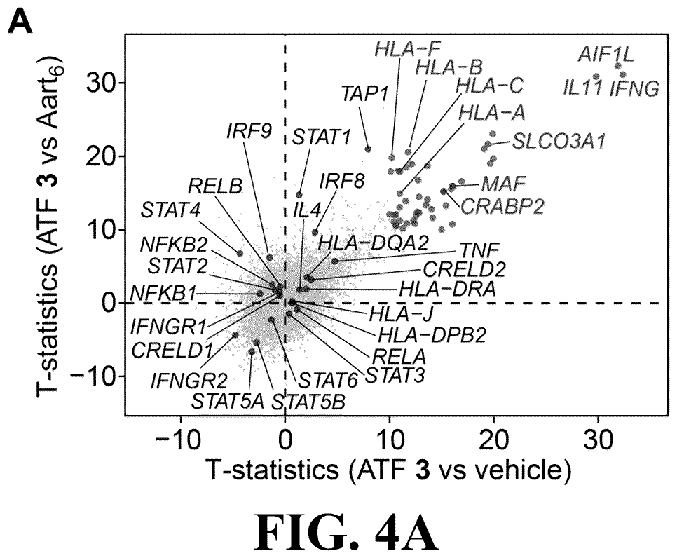

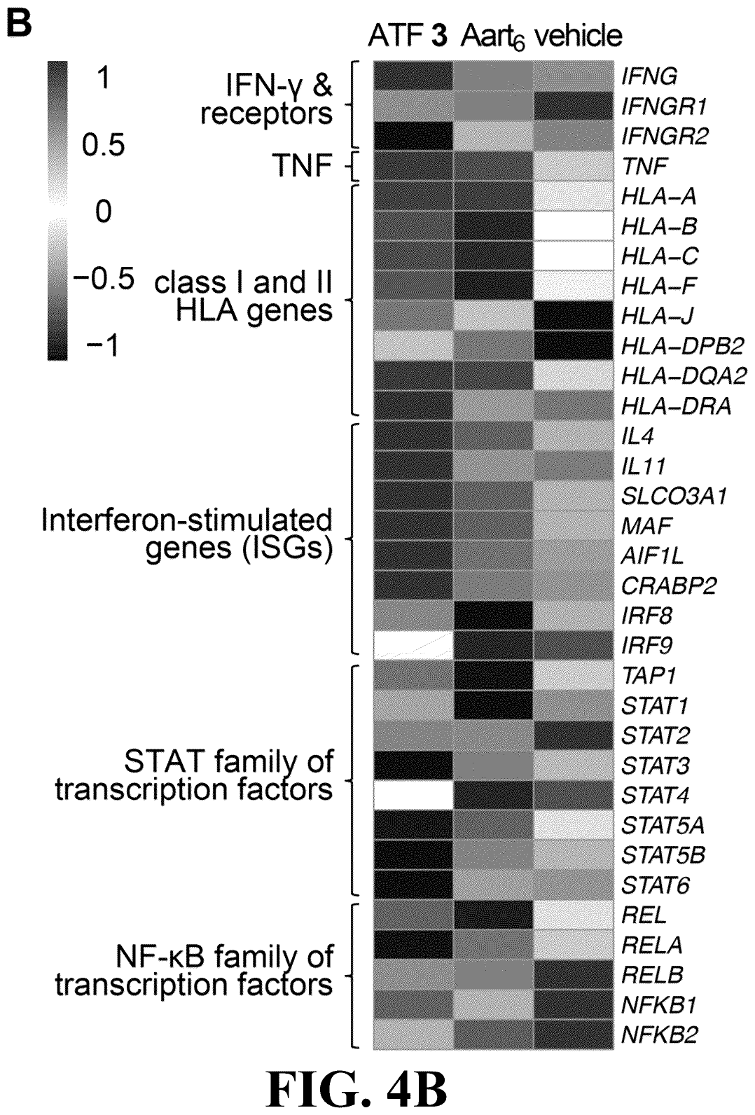

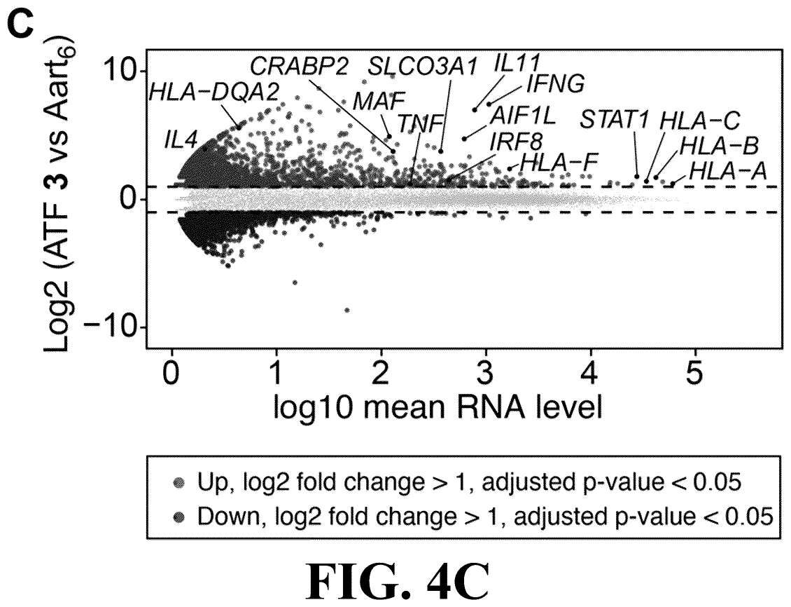

The biological response from ATF 3 was assessed based on the transcriptomic profile of ATF-treated Jurkat cells. Due to the significant expression of IFN-γ observed for ATF 3, without wishing to be bound by theory, it was hypothesized that this construct would also induce upregulation of the hIFNG gene and associated interferon-stimulated genes (ISGs). The activity was determined by evaluating RNA-sequencing (RNA-seq) results from Jurkat cells transfected ATF 3, and comparing these results to cells transfected with buffer ‘vehicle’ and a homologous ATF called Aart₆. Differential gene expression (DGE) analysis of the total RNA showed upregulated expression of the target hIFNG gene in cells treated with ATF 3, indicating a successful activation of the target gene (FIG. 4A).

Signal transduction from IFN-γ stimulates the expression of class I and II human leukocyte antigens (HLAs), and also the STAT and IFN regulatory factor (IRF) families of transcription factors. In Jurkat cells treated with ATF 3, the transcriptomic profile shows upregulated expression of STAT1 and HLA genes, including HLA-A, HLA-B, HLA-C, HLA-DRA, and HLA-F. The transcriptomic profile also shows upregulated genes for inflammatory proteins, including IL11 (IL-11) and AIFIL (allograft inflammatory factor 1), and for NF-κB proteins, including SLCO3A1 (solute carrier organic anion transporter family member 3A1), MAF (musculoaponeurotic fibrosarcoma), and CRABP2 (cellular retinoic acid-binding protein 2). The upregulation of these genes supports the observation that ATF 3 mediates IFN-γ expression and, in turn, promotes signal transduction for inflammatory responses.

### Example 5

### Preparation of a Recombinant Artificial Transcription Factor

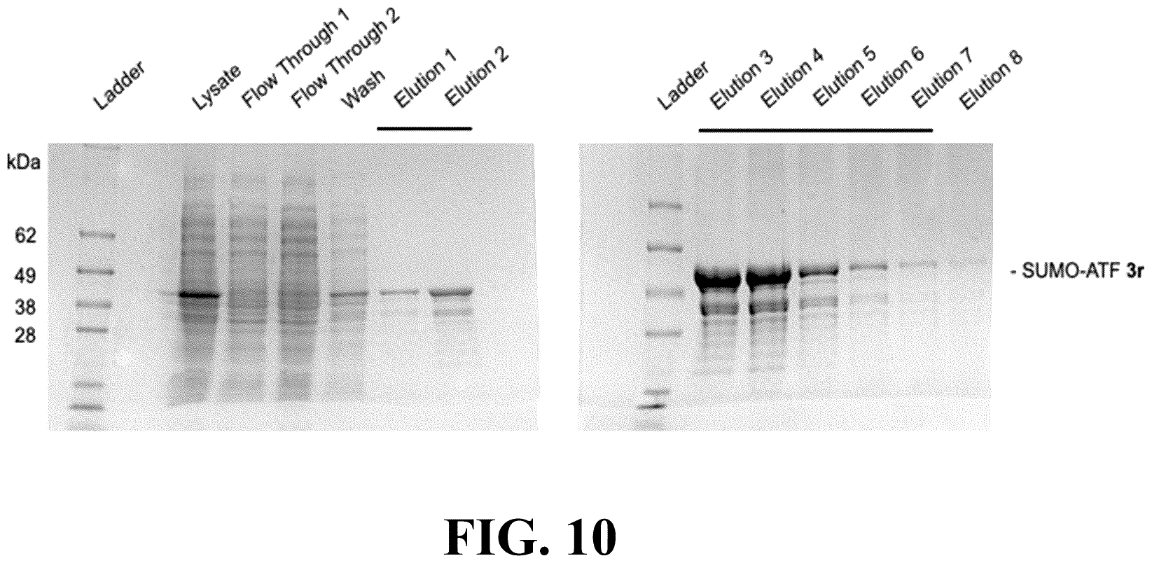

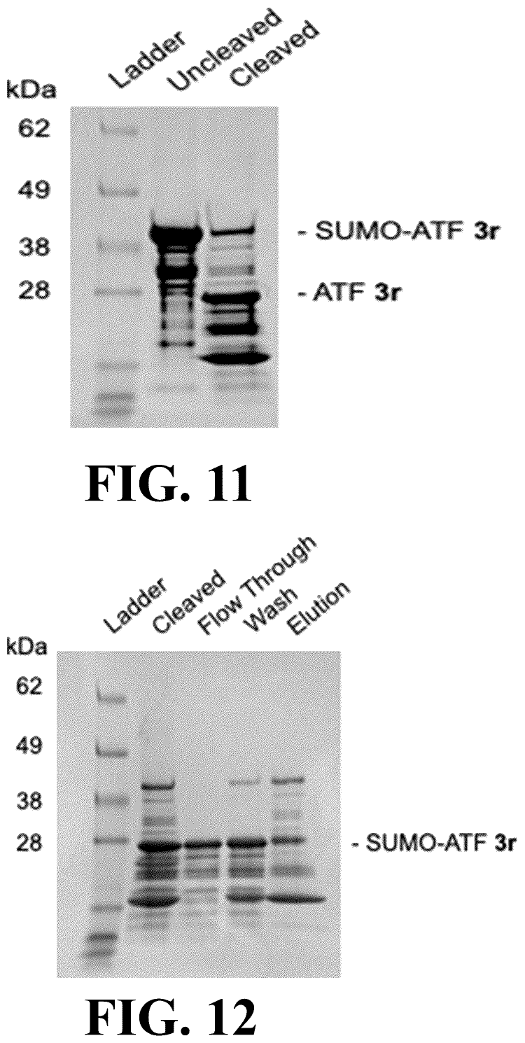

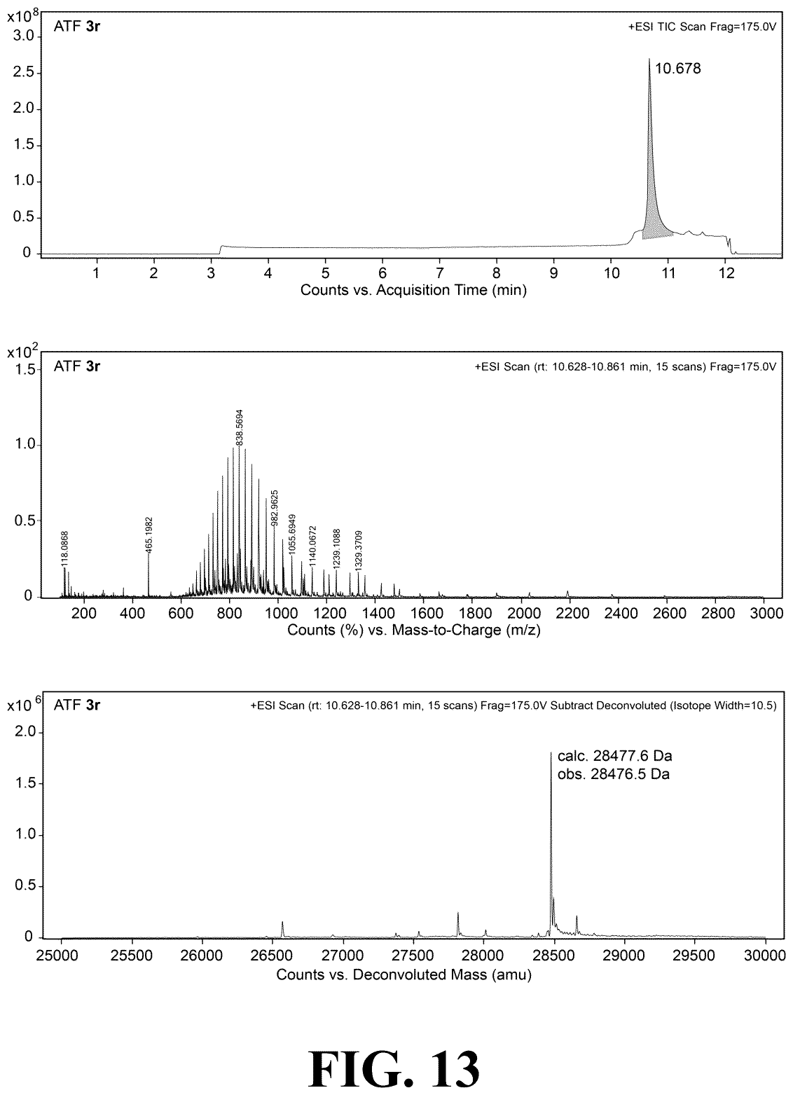

A recombinant form of ATF 3, called ATF 3r, was isolated to enable biophysical characterization of the protein (Tables 6-7). ATF 3r was expressed as a fusion protein termed SUMO-ATF 3r, purified by Ni NTA affinity chromatography, and treated with the Ulp1 cysteine protease. ATF 3r was obtained after RP-HPLC purification and lyophilization, which gave the protein in pure form as a dry powder (trifluoroacetic acid salt). The identity of the protein was confirmed by SDS-PAGE and LC-MS (FIGS. 10-13), and further characterized to evaluate DNA recognition, protein folding, and thermal stability.

TABLE 6
Amino acid sequence for the hIFNG-
targeting protein SUMO-ATF 3r.

| | Amino Acid Sequence¹ |
|---|---|
| SUMO-ATF 3r | MGSSHHHHHHGSGLVPRGSASMSDSEVNQ |
| (SEQ ID NO: 41) | EAKPEVKPEVKPETHINLKVSDGSSEIF |
| | FKIKKTTPLRRIMEAFAKRQGKEMDSLR |
| | FLYDGIRIQADQTPEDLDMEDNDIIEAH |
| | REQIGGMLEPGEKPYKCPECGKSFSDPG |
| | HLVRHQRTHTGEKPYKCPECGKSFSRSD |
| | ELVRHQRTHTGEKPYKCPECGKSFSRAD |
| | NLTEHQRTHTGEKPYKCPECGKSFSQSG |
| | DLRRHQRTHTGEKPYKCPECGKSFSRRD |
| | ELNVHQRTHTGEKPYKCPECGKSFSQLA |
| | HLRAHQRTHTGKKTSPKKKRKVEASGSG |
| | RADALDDFDLDMLGSDALDDFDLDMLGS |
| | DALDDFDLDMLGSDALDDEDLDMLYPYD |
| | VPDYA |

¹Underlined sequence indicates the SUMO tag which is cleaved by Ulp-1.

TABLE 7
Nucleotide sequences for the hIFNG-targeting
protein SUMO-ATF 3r.

| | Nucleotide Sequence¹ |
|---|---|
| SUMO-ATF 3r | AATAATTTTGTTTAACTTTAAGAAG |
| (SEQ ID NO: 42) | GAGATATACATATGGGCAGCAGCC |
| | ATCATCATCATCATCACGGCAGCG |
| | GCCTGGTGCCGCGCGGCAGCGCTA |
| | GCATGTCGGACTCAGAAGTCAATC |
| | AAGAAGCTAAGCCAGAGGTCAAGC |
| | CAGAAGTCAAGCCTGAGACTCACA |
| | TCAATTTAAAGGTGTCCGATGGAT |
| | CTTCAGAGATCTTCTTCAAGATCA |
| | AAAAGACCACTCCTTTAAGAAGGC |
| | TGATGGAAGCGTTCGCTAAAAGAC |
| | AGGGTAAGGAAATGGACTCCTTAA |
| | GATTCTTGTACGACGGTATTAGAA |
| | TTCAAGCTGATCAGACCCCTGAAG |
| | ATTTGGACATGGAGGATAACGATA |
| | TTATTGAGGCTCACAGAGAACAGA |
| | TTGGTGGTATGCTGGAACCAGGGG |
| | AAAAACCATACAAATGCCCAGAGT |
| | GCGGAAAGTCATTTTCGGACCCCG |
| | GTCACCTGGTCCGCCACCAACGGA |
| | CACATACGGGCGAAAAGCCCTATA |
| | AGTGCCCTGAATGTGGCAAAAGTT |
| | TCTCGAGATCCGATGAGCTTGTTC |
| | GGCACCAACGTACGCACACTGGAG |
| | AAAAACCTTACAAATGTCCTGAGT |
| | GCGGTAAATCATTCAGTCGGGCGG |
| | ACAACCTGACAGAGCATCAGAGAA |
| | CTCACACTGGAGAGAAGCCATATA |
| | AATGCCCGGAATGTGGTAAGTCTT |
| | TTTCTCAATCTGGTGATTTACGTC |
| | GTCATCAGCGCACGCATACGGGTG |
| | AGAAGCCTTATAAGTGCCCCGAAT |
| | GCGGCAAGAGCTTTTCCCGGCGGG |
| | ACGAGTTGAACGTGCACCAACGGA |
| | CCCACACAGGAGAAAAACCGTATA |
| | AGTGCCCGGAGTGCGGTAAGAGTT |
| | TTTCCCAACTTGCACATCTTCGCG |
| | CTCATCAGCGTACCCATACTGGTA |
| | AGAAGACAAGCCCCAAGAAGAAGA |
| | GGAAGGTGGAGGCCAGCGGTTCCG |
| | GACGGGCTGACGCATTGGACGATT |
| | TTGATCTGGATATGCTGGGAAGTG |
| | ACGCCCTCGATGATTTTGACCTTG |
| | ACATGCTTGGTTCGGATGCCCTTG |
| | ATGACTTTGACCTCGACATGCTCG |
| | GCAGTGACGCCCTTGATGATTTCG |
| | ACCTGGACATGCTGTACCCCTACG |
| | ATGTACCGGATTACGCTTGATAAA |

¹Underlined sequence indicates the SUMO tag which is cleaved by Ulp-1.

### Example 6

Assessing ATF Recognition of the Target hIFNG Gene

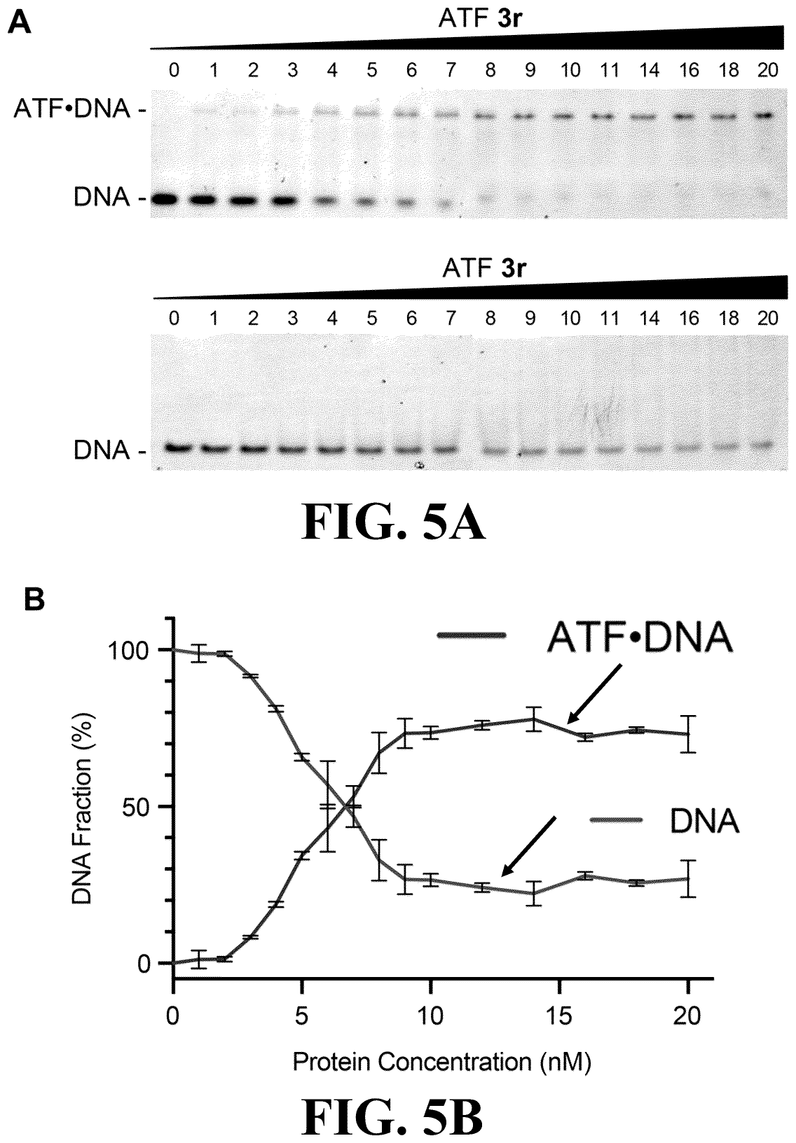

ATF recognition of DNA strands is mediated by the ZF proteins. To evaluate the gene recognition properties of ATF 3r, electrophoretic mobility shift assays (EMSAs) were used to determine the binding selectivity, affinity, and stoichiometry (FIGS. 5A-5B). Prior binding studies of other zinc finger proteins containing six subunits have shown K_{D} values that range from 2 to 15 nM. ATF 3r contains six subunits but also an activation domain. Nonetheless, the EMSA studies show that ATF 3r maintains effective and selective binding to the target region.

The DNA-binding properties was evaluated with synthetic double-stranded (dsDNA) fragments encoding the target hIFNG sequence and an irrelevant sequence as a negative control. ATF 3r protein was incubated with each DNA fragment, followed by separation with native PAGE and visualization with a SYBR Safe DNA stain (FIG. 5A). Mixtures of ATF 3r with the target DNA showed a second row of bands appear at higher protein concentrations, corresponding to a 1:1 ATF:DNA complex. Furthermore, the band intensities from the free DNA decrease at higher protein concentrations. Integration of the DNA band intensities shows that 50% is bound to protein at a 6:1 ratio of ATF 3r to DNA (FIG. 5B). The fraction bound was further plotted to approximate a K_{D}. Fitting the data to a 1:1 binding model showed an apparent K_{D} of 5.2±0.3 nM. This K_{D} is comparable with previously reported values of zinc finger proteins and establishes that ATF 3r binds within the known range of six-linked zinc finger proteins. To further demonstrate the selectivity of ATF 3r, the protein was mixed with an irrelevant DNA strand and evaluated (FIG. 5A). EMSA showed a single set of horizontal bands that correspond only with free DNA, indicating that ATF 3r does not recognize the irrelevant DNA sequence.

### Example 7

### Assessing ATF Folding and Thermal Stability

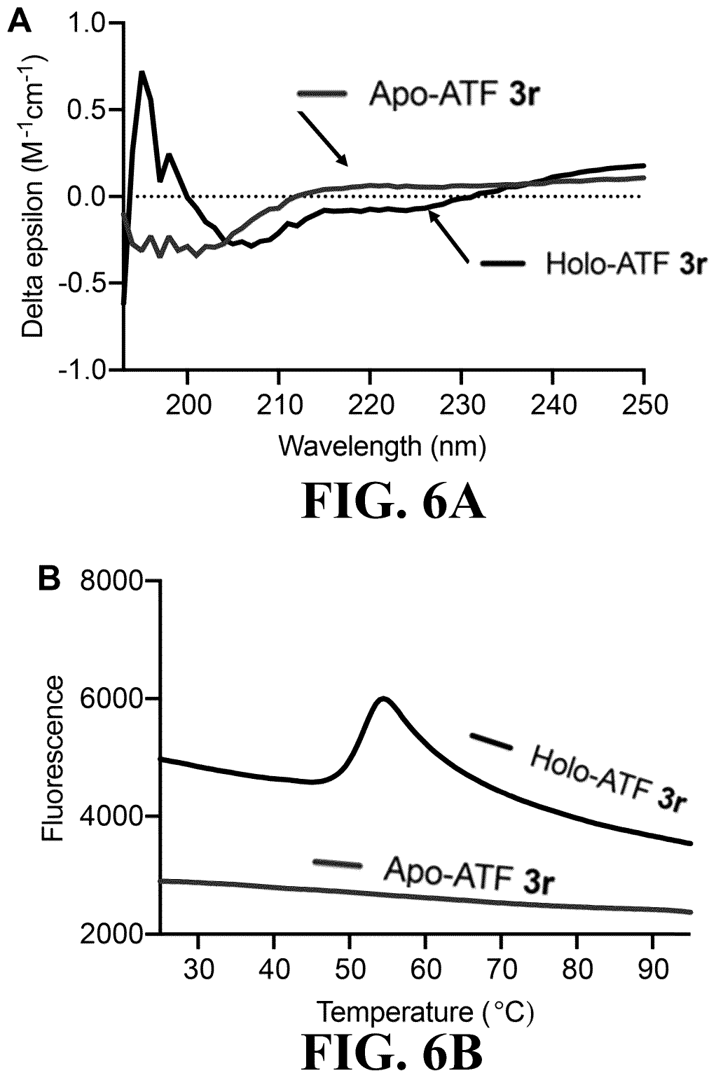

Circular dichroism (CD) and differential scanning fluorimetry (DSF) experiments were performed to evaluate ZF folding and resistance to thermal denaturation. These experiments established that ATF 3r adopts a folded structure that is stable under biological temperatures (FIGS. 6A-6B). The zinc finger sequences within each ATF belong to the Cys₂His₂ family, which is characterized by adopting a ββα fold that is stabilized by a zinc ion. Performing CD analysis on the apo- and holoprotein provides valuable information on protein folding. CD analysis shows significant differences between ATF 3r folding in the apo- and holo-forms (FIG. 6A). Apo-ATF 3r represents an unfolded protein with a negative peak centered around 200 nm, corresponding to random coils. Holo-ATF 3r depicts a folded protein that has ββα characteristics. Folding of holo-ATF 3r is demonstrated by the negative peaks at 205 and 220 nm, along with a positive peak at 200 nm. The protein characteristics depicted in the CD data are comparable to previous studies on similar zinc finger proteins.

Thermal stability is an important feature when considering therapeutic applications under biological conditions. DSF quantifies thermal stability by providing a melting temperature (Tₘ) that can be compared to other proteins. Beyond determining the thermal stability of ATF 3r, it was also observed that zinc ions are necessary for ATF protein folding. The Tₘ was determined by obtaining the first derivative of the DSF spectra and identifying the point of inflection (FIG. 6B). The DSF analysis shows that the holo-ATF 3r exhibits a Tₘ of 52° C., but apo-ATF 3r does not adopt a folded structure.

### Example 8

### Activation of Tumor Necrosis Factor (TNF) Expression

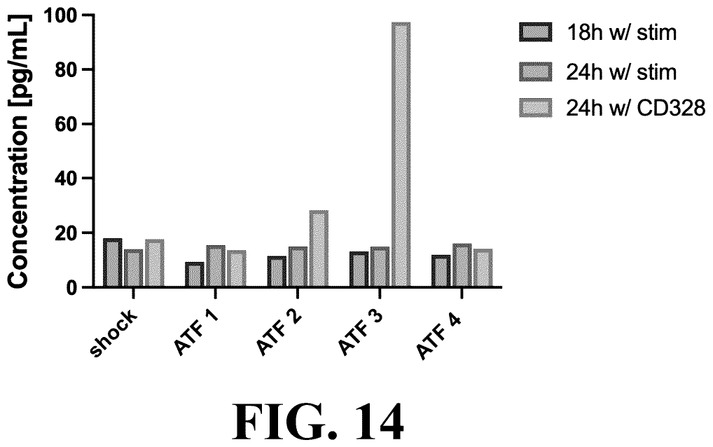

The activity of each ATF was established in Jurkat T cells through measurement of TNF expression using ELISAs. ATFs 1-4 were evaluated for their ability to mediate TNF expression compared to the vehicle control (‘shock’, electroporation with no plasmid). In a time-course ELISA (FIG. 14) cells were collected at 18 and 24 hours post transfection and the supernatant tested alongside a shock control. In this experiment, a large increase in TNF expression by Jurkat T cells following treatment with ATF 3 and CD3/CD28 Dynabeads was observed. ATF 3 treatment with Dynabeads shows a concentration around 100 pg/mL relative to the levels in the shock, which is about a 5-fold increase. While ATF 2 treatment with Dynabeads did show moderate increase in TNF expression, all other conditions and timepoints are comparable to shock baseline values.

In a subsequent Jurkat T cell transfection with stimulation cocktail treatment at one hour and cell lysis at 24 hours post transfection, brefeldin A (BFA) was added at one hour as well. BFA inhibits protein transport out of cells, resulting in produced TNF protein remaining inside of the cells for subsequent analysis rather than in the media. This allows, without wishing to be bound by theory, for analysis of cell lysis only while discarding the sample supernatant.

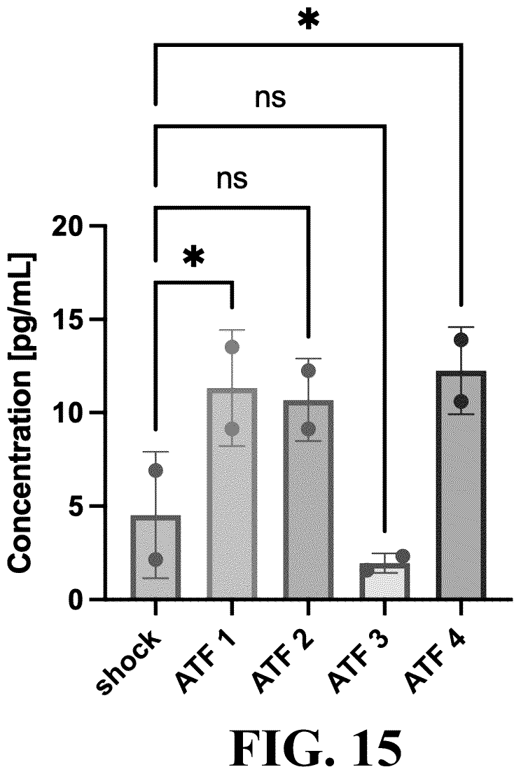

In the ELISA, at 24 hours post transfection with BFA included in the treatment conditions, ATFs 1, 3, and 4 showed increased TNF expression compared to the shock control (FIG. 15). Of these, ATFs 1 and 3 had statistically significant increases in TNF production, with ATF 3 inducing about a 3-fold increase compared to shock values. ATF 2 showed decreased TNF production, indicating that either the gene target naturally inhibits TNF expression or that TNF production was potent after treatment and resulted in cell death as part of a rigorous immune response.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A method for treating a disease characterized by immune dysfunction, wherein the method comprises administering to a subject in need thereof a therapeutically effective amount of an artificial transcription factor comprising:
a DNA-binding domain comprising from about 2 to about 8 zinc finger proteins; and
an activator domain,
wherein the zinc finger proteins bind to a target site that is a functionally equivalent variant at least 90% identical to SEQ ID NOs: 5-28, 30-34, 41-66, and/or 67-79.

2. The method of claim 1, wherein the DNA-binding domain comprises from about 3 to about 6 zinc finger proteins.

3. The method of claim 1, wherein the immune-related disease is cancer.

4. The method of claim 1, wherein the cancer is selected from the group consisting of melanoma, non-small cell lung cancer (NSCLC), triple-negative breast cancer (TNBC), renal cell carcinoma, glioblastoma, or colorectal cancer.

5. The method of claim 1, wherein the immune-related disease is an autoimmune disease selected from multiple sclerosis, rheumatoid arthritis, inflammatory bowel disease, or systemic lupus erythematosus.

6. The method of claim 1, wherein the immune-related disease is a chronic infectious disease selected from human immunodeficiency virus (HIV), hepatitis B virus (HBV), hepatitis C virus (HCV), or tuberculosis.

7. The method of claim 1, wherein the activator domain is derived from one or more viral proteins.

8. The method of claim 7, wherein the one or more viral protein is selected from a group consisting of Herpes Simplex Viral Protein 16 (VP16), Herpes Simplex Viral Protein 64 (VP64), VP128, p65, p300, or a combination thereof.

9. The method of claim 1, wherein the activator domain comprises a nuclear localization signal (NLS).

10. The method of claim 9, wherein the NLS is encoded by an amino acid sequence comprising SEQ ID NO: 38.

11. The method of claim 1, wherein the activator domain comprises a protein tag.

12. The method of claim 11, wherein the protein tag comprises a hemagglutinin A (HA) tag, FLAG, or a combination thereof.

13. The method of claim 1, wherein the artificial transcription factor further comprises a fluorescent domain.

14. The method of claim 13, wherein the fluorescent domain comprises a fluorescent protein selected from a group consisting of AcGFP, TurboGFP, Emerald, Azami Green, ZsGreen, EBFP, Sapphire, T-Sapphire, ECFP, mCFP, Cerulean, CyPet, AmCyan1, Midori-Ishi Cyan, mTFP1 (Teal), enhanced yellow fluorescent protein (EYFP), Topaz, Venus, mCitrine, YPet, PhiYFP, ZsYellow1, mBanana, Kusabira Orange, mOrange, dTomato, dTomato-Tandem, Discosoma red (DsRed), DsRed2, DsRed-Express (T1), DsRed-Monomer, mTangerine, mStrawberry, AsRed2, mRFP1, JRed, mCherry, HcRed1, mRaspberry, HcRed1, HcRed-Tandem, mPlum, and AQ143.

15. A method for activating expression of an immune signaling gene in a cell, the method comprising contacting the cell with an artificial transcription factor comprising:
a DNA-binding domain comprising from about 2 to about 8 zinc finger proteins; and
a transcriptional activator domain,
wherein the zinc finger proteins bind to a target site that is a functionally equivalent variant at least 90% identical to SEQ ID NOs: 5-28, 30-34, 41-66, and/or 67-79 to mediate transcriptional activation.

16. The method of claim 15, wherein the artificial transcription factor upregulates expression of the gene at least 2 fold in the cell compared to a control.

17. The method of claim 15, wherein the artificial transcription factor upregulates expression of the immune signaling gene comprises interleukin-2 (IL-2), IL-7, IL-12, IL-15, IL-21, interferon gamma (IFN-γ), tumor necrosis factor alpha (TNF-α), tumor necrosis factor beta (TNF-β), or granulocyte-macrophage colony-stimulating factor (GM-CSF).

18. The method of claim 15, wherein the artificial transcription factor comprises SEQ ID NOs: 30, 31, 32, 33, or 41, which correspond to gene-encoded ATFs for modulating gene expression and functionally equivalent variants.

19. The method of claim 15, wherein the artificial transcription factor comprises SEQ ID NOs: 32 or 41, which correspond to recombinant ATF proteins for modulating gene expression and top-performing variants.

20. The method of claim 15, wherein the artificial transcription factor comprises SEQ ID NO: 34.


SEQUENCE LISTING (85 sequences)

SEQ ID NO: 1 - DNA, 18 bp, Homo sapiens
  source 1..18: mol_type = genomic DNA
cttgtgaaaatacgtaat

SEQ ID NO: 2 - DNA, 18 bp, Homo sapiens
  source 1..18: mol_type = genomic DNA
gaaactctaactacaaca

SEQ ID NO: 3 - DNA, 18 bp, Homo sapiens
  source 1..18: mol_type = genomic DNA
agaatggcacaggtgggc

SEQ ID NO: 4 - DNA, 18 bp, Homo sapiens
  source 1..18: mol_type = genomic DNA
atggtgtgaagtaaaagt

SEQ ID NO: 5 - protein, 7 aa, synthetic construct
TTGNLTV

SEQ ID NO: 6 - protein, 7 aa, synthetic construct
SRRTCRA

SEQ ID NO: 7 - protein, 7 aa, synthetic construct
QKSSLIA

SEQ ID NO: 8 - protein, 7 aa, synthetic construct
QRANLRA

SEQ ID NO: 9 - protein, 7 aa, synthetic construct
RSDELVR

SEQ ID NO: 10 - protein, 7 aa, synthetic construct
TTGALTE

SEQ ID NO: 11 - protein, 7 aa, synthetic construct
SPADLTR

SEQ ID NO: 12 - protein, 7 aa, synthetic construct
SPADLTR

SEQ ID NO: 13 - protein, 7 aa, synthetic construct
THLDLIR

SEQ ID NO: 14 - protein, 7 aa, synthetic construct
QNSTLTE

SEQ ID NO: 15 - protein, 7 aa, synthetic construct
THLDLIR

SEQ ID NO: 16 - protein, 7 aa, synthetic construct
QSSNLVR

SEQ ID NO: 17 - protein, 7 aa, synthetic construct
DPGHLVR

SEQ ID NO: 18 - protein, 7 aa, synthetic construct
RSDELVR

SEQ ID NO: 19 - protein, 7 aa, synthetic construct
RADNLTE

SEQ ID NO: 20 - protein, 7 aa, synthetic construct
QSGDLRR

SEQ ID NO: 21 - protein, 7 aa, synthetic construct
RRDELNV

SEQ ID NO: 22 - protein, 7 aa, synthetic construct
QLAHLRA

SEQ ID NO: 23 - protein, 7 aa, synthetic construct
HRTTLTN

SEQ ID NO: 24 - protein, 7 aa, synthetic construct
QRANLRA

SEQ ID NO: 25 - protein, 7 aa, synthetic construct
HRTTLTN

SEQ ID NO: 26 - protein, 7 aa, synthetic construct
QAGHLAS

SEQ ID NO: 27 - protein, 7 aa, synthetic construct
RSDELVR

SEQ ID NO: 28 - protein, 7 aa, synthetic construct
RRDELNV

SEQ ID NO: 29 - protein, 176 aa, synthetic construct
  VARIANT 19: note = Any amino acid
  VARIANT 20: note = Any amino acid
  VARIANT 21: note = Any amino acid
  VARIANT 22: note = Any amino acid
  VARIANT 23: note = Any amino acid
  VARIANT 24: note = Any amino acid
  VARIANT 25: note = Any amino acid
  VARIANT 47: note = Any amino acid
  VARIANT 48: note = Any amino acid
  VARIANT 49: note = Any amino acid
  VARIANT 50: note = Any amino acid
  VARIANT 51: note = Any amino acid
  VARIANT 52: note = Any amino acid
  VARIANT 53: note = Any amino acid
  VARIANT 75: note = Any amino acid
  VARIANT 76: note = Any amino acid
  VARIANT 77: note = Any amino acid
  VARIANT 78: note = Any amino acid
  VARIANT 79: note = Any amino acid
  VARIANT 80: note = Any amino acid
  VARIANT 81: note = Any amino acid
  VARIANT 104: note = Any amino acid
  VARIANT 105: note = Any amino acid
  VARIANT 106: note = Any amino acid
  VARIANT 107: note = Any amino acid
  VARIANT 108: note = Any amino acid
  VARIANT 109: note = Any amino acid
  VARIANT 131: note = Any amino acid
  VARIANT 132: note = Any amino acid
  VARIANT 133: note = Any amino acid
  VARIANT 134: note = Any amino acid
  VARIANT 135: note = Any amino acid
  VARIANT 136: note = Any amino acid
  VARIANT 137: note = Any amino acid
  VARIANT 159: note = Any amino acid
  VARIANT 160: note = Any amino acid
  VARIANT 161: note = Any amino acid
  VARIANT 162: note = Any amino acid
  VARIANT 163: note = Any amino acid
  VARIANT 164: note = Any amino acid
  VARIANT 165: note = Any amino acid
LEPGEKPYKCPECGKSFSXXXXXXXHQRTHTGEKPYKCPECGKSFSXXXXXXXHQRTHTGEKPYKCPECGKSFSXXXXXXXHQRTHTGEKPYKCPECGKSFSSXXXXXXHQRTHTGEKPYKCPECGKSFSXXXXXXXHQRTHTGEKPYKCPECGKSFSXXXXXXXHQRTHTGKKTS

SEQ ID NO: 30 - protein, 496 aa, synthetic construct
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGSFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERAEGRHHLFLGSGEGRGSLLTCGDVEENPGPLEPGEKPYKCPECGKSFSTTGNLTVHQRTHTGEKPYKCPECGKSFSSRRTCRAHQRTHTGEKPYKCPECGKSFSQKSSLIAHQRTHTGEKPYKCPECGKSFSQRANLRAHQRTHTGEKPYKCPECGKSFSRSDELVRHQRTHTGEKPYKCPECGKSFSTTGALTEHQRTHTGKKTSPKKKRKVEASGSGRADALDDFDLDMLGSDALDDFDLDMLGSDALDDFDLDMLGSDALDDFDLDMLYPYDVPDYA

SEQ ID NO: 31 - protein, 496 aa, synthetic construct
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGSFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERAEGRHHLFLGSGEGRGSLLTCGDVEENPGPLEPGEKPYKCPECGKSFSSPADLTRHQRTHTGEKPYKCPECGKSFSSPADLTRHQRTHTGEKPYKCPECGKSFSTHLDLIRHQRTHTGEKPYKCPECGKSFSQNSTLTEHQRTHTGEKPYKCPECGKSFSTHLDLIRHQRTHTGEKPYKCPECGKSFSQSSNLVRHQRTHTGKKTSPKKKRKVEASGSGRADALDDFDLDMLGSDALDDFDLDMLGSDALDDFDLDMLGSDALDDFDLDMLYPYDVPDYA

SEQ ID NO: 32 - protein, 496 aa, synthetic construct
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGSFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERAEGRHHLFLGSGEGRGSLLTCGDVEENPGPLEPGEKPYKCPECGKSFSDPGHLVRHQRTHTGEKPYKCPECGKSFSRSDELVRHQRTHTGEKPYKCPECGKSFSRADNLTEHQRTHTGEKPYKCPECGKSFSQSGDLRRHQRTHTGEKPYKCPECGKSFSRRDELNVHQRTHTGEKPYKCPECGKSFSQLAHLRAHQRTHTGKKTSPKKKRKVEASGSGRADALDDFDLDMLGSDALDDFDLDMLGSDALDDFDLDMLGSDALDDFDLDMLYPYDVPDYA

SEQ ID NO: 33 - protein, 496 aa, synthetic construct
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGSFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERAEGRHHLFLGSGEGRGSLLTCGDVEENPGPLEPGEKPYKCPECGKSFSDKKDLTRHQRTHTGEKPYKCPECGKSFSQSSSLVRHQRTHTGEKPYKCPECGKSFSQRAHLERHQRTHTGEKPYKCPECGKSFSRKDNLKNHQRTHTGEKPYKCPECGKSFSQRANLRAHQRTHTGEKPYKCPECGKSFSQKSSLIAHQRTHTGKKTSPKKKRKVEASGSGRADALDDFDLDMLGSDALDDFDLDMLGSDALDDFDLDMLGSDALDDFDLDMLYPYDVPDYA

SEQ ID NO: 34 - protein, 510 aa, synthetic construct
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGSFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERAEGRHHLFLGSGEGRGSLLTCGDVEENPGPISEFGSSSSVAQAALEPGEKPYACPECGKSFSRSDHLAEHQRTHTGEKPYKCPECGKSFSDKKDLTRHQRTHTGEKPYKCPECGKSFSQRANLRAHQRTHTGEKPYACPECGKSFSQLAHLRAHQRTHTGEKPYKCPECGKSFSREDLNHTHQRTHTGEKPYKCPECGKSFSRRDALNVHQRTHTGKKTSPKKKKKVEASGSGRADALDDFDLDMLGSDALDDFDLDMLGSDALDDFDLDMLGSDALDDFDLDMLYPYDVPDYA

SEQ ID NO: 35 - protein, 225 aa, synthetic construct
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGSFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERAEGRHHLFL

SEQ ID NO: 36 - skipped

SEQ ID NO: 37 - protein, 18 aa, synthetic construct
EGRGSLLTCGDVEENPGP

SEQ ID NO: 38 - protein, 7 aa, synthetic construct
PKKKRKV

SEQ ID NO: 39 - protein, 50 aa, synthetic construct
DALDDFDLDMLGSDALDDFDLDMLGSDALDDFDLDMLGSDALDDFDLDML

SEQ ID NO: 40 - protein, 9 aa, synthetic construct
YPYDVPDYA

SEQ ID NO: 41 - protein, 370 aa, synthetic construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMLEPGEKPYKCPECGKSFSDPGHLVRHQRTHTGEKPYKCPECGKSFSRSDELVRHQRTHTGEKPYKCPECGKSFSRADNLTEHQRTHTGEKPYKCPECGKSFSQSGDLRRHQRTHTGEKPYKCPECGKSFSRRDELNVHQRTHTGEKPYKCPECGKSFSQLAHLRAHQRTHTGKKTSPKKKRKVEASGSGRADALDDFDLDMLGSDALDDFDLDMLGSDALDDFDLDMLGSDALDDFDLDMLYPYDVPDYA

SEQ ID NO: 42 - DNA, 1153 bp, synthetic construct
  source 1..1153: mol_type = other DNA
aataattttgtttaactttaagaaggagatatacatatgggcagcagccatcatcatcatcatcacggcagcggcctggtgccgcgcggcagcgctagcatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtggtatgctggaaccaggggaaaaaccatacaaatgcccagagtgcggaaagtcattttcggaccccggtcacctggtccgccaccaacggacacatacgggcgaaaagccctataagtgccctgaatgtggcaaaagtttctcgagatccgatgagcttgttcggcaccaacgtacgcacactggagaaaaaccttacaaatgtcctgagtgcggtaaatcattcagtcgggcggacaacctgacagagcatcagagaactcacactggagagaagccatataaatgcccggaatgtggtaagtctttttctcaatctggtgatttacgtcgtcatcagcgcacgcatacgggtgagaagccttataagtgccccgaatgcggcaagagcttttcccggcgggacgagttgaacgtgcaccaacggacccacacaggagaaaaaccgtataagtgcccggagtgcggtaagagtttttcccaacttgcacatcttcgcgctcatcagcgtacccatactggtaagaagacaagccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtacccctacgatgtaccggattacgcttgataaa

SEQ ID NO: 43 - protein, 7 aa, synthetic construct
ERSHLRE

SEQ ID NO: 44 - protein, 7 aa, synthetic construct
SKKHLAE

SEQ ID NO: 45 - protein, 7 aa, synthetic construct
SPADLTR

SEQ ID NO: 46 - protein, 7 aa, synthetic construct
DPGHLVR

SEQ ID NO: 47 - protein, 7 aa, synthetic construct
TSGSLVR

SEQ ID NO: 48 - protein, 7 aa, synthetic construct
TSGSLVR

SEQ ID NO: 49 - protein, 7 aa, synthetic construct
HTGHLLE

SEQ ID NO: 50 - protein, 7 aa, synthetic construct
SRGNLKS

SEQ ID NO: 51 - protein, 7 aa, synthetic construct
SKKALTE

SEQ ID NO: 52 - protein, 7 aa, synthetic construct
DCRDLAR

SEQ ID NO: 53 - protein, 7 aa, synthetic construct
RSDHLTN

SEQ ID NO: 54 - protein, 7 aa, synthetic construct
APKALGW

SEQ ID NO: 55 - protein, 7 aa, synthetic construct
RTDTLRD

SEQ ID NO: 56 - protein, 7 aa, synthetic construct
RSDKLVR

SEQ ID NO: 57 - protein, 7 aa, synthetic construct
DPGHLVR

SEQ ID NO: 58 - protein, 7 aa, synthetic construct
RRDELNV

SEQ ID NO: 59 - protein, 7 aa, synthetic construct
RSDHLTN

SEQ ID NO: 60 - protein, 7 aa, synthetic construct
RKDALRG

SEQ ID NO: 61 - protein, 7 aa, synthetic construct
QSGHLTE

SEQ ID NO: 62 - protein, 7 aa, synthetic construct
HRTTLTN

SEQ ID NO: 63 - protein, 7 aa, synthetic construct
QRAHLER

SEQ ID NO: 64 - protein, 7 aa, synthetic construct
ARGNLRT

SEQ ID NO: 65 - protein, 7 aa, synthetic construct
DPGHLVR

SEQ ID NO: 66 - protein, 7 aa, synthetic construct
RADNLTE

SEQ ID NO: 67 - protein, 496 aa, synthetic construct
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGSFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERAEGRHHLFLGSGEGRGSLLTCGDVEENPGPLEPGEKPYKCPECGKSFSERSHLREHQRTHTGEKPYKCPECGKSFSSKKHLAEHQRTHTGEKPYKCPECGKSFSSPADLTRHQRTHTGEKPYKCPECGKSFSDPGHLVRHQRTHTGEKPYKCPECGKSFSTSGSLVRHQRTHTGEKPYKCPECGKSFSTSGSLVRHQRTHTGKKTSPKKKRKVEASGSGRADALDDFDLDMLGSDALDDFDLDMLGSDALDDFDLDMLGSDALDDFDLDMLYPYDVPDYA

SEQ ID NO: 68 - protein, 496 aa, synthetic construct
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGSFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERAEGRHHLFLGSGEGRGSLLTCGDVEENPGPLEPGEKPYKCPECGKSFSHTGHLLEHQRTHTGEKPYKCPECGKSFSSRGNLKSHQRTHTGEKPYKCPECGKSFSSKKALTEHQRTHTGEKPYKCPECGKSFSDCRDLARHQRTHTGEKPYKCPECGKSFSRSDHLTNHQRTHTGEKPYKCPECGKSFSAPKALGWHQRTHTGKKTSPKKKRKVEASGSGRADALDDFDLDMLGSDALDDFDLDMLGSDALDDFDLDMLGSDALDDFDLDMLYPYDVPDYA

SEQ ID NO: 69 - protein, 496 aa, synthetic construct
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGSFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERAEGRHHLFLGSGEGRGSLLTCGDVEENPGPLEPGEKPYKCPECGKSFSRTDTLRDHQRTHTGEKPYKCPECGKSFSRSDKLVRHQRTHTGEKPYKCPECGKSFSDPGHLVRHQRTHTGEKPYKCPECGKSFSRRDELNVHQRTHTGEKPYKCPECGKSFSRSDHLTNHQRTHTGEKPYKCPECGKSFSRKDALRGHQRTHTGKKTSPKKKRKVEASGSGRADALDDFDLDMLGSDALDDFDLDMLGSDALDDFDLDMLGSDALDDFDLDMLYPYDVPDYA

SEQ ID NO: 70 - protein, 496 aa, synthetic construct
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGSFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERAEGRHHLFLGSGEGRGSLLTCGDVEENPGPLEPGEKPYKCPECGKSFSQSGHLTEHQRTHTGEKPYKCPECGKSFSHRTTLTNHQRTHTGEKPYKCPECGKSFSQRAHLERHQRTHTGEKPYKCPECGKSFSARGNLRTHQRTHTGEKPYKCPECGKSFSDPGHLVRHQRTHTGEKPYKCPECGKSFSRADNLTEHQRTHTGKKTSPKKKRKVEASGSGRADALDDFDLDMLGSDALDDFDLDMLGSDALDDFDLDMLGSDALDDFDLDMLYPYDVPDYA

SEQ ID NO: 71 - DNA, 1494 bp, synthetic construct
  source 1..1494: mol_type = other DNA
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctggggtcaggagagggcagaggaagtcttctaacatgcggtgacgtggaggagaatcccggccctctggaacccggggaaaagccatacaaatgcccagaatgtggaaaatctttcagcacaaccggtaaccttacggtacaccagagaactcacacaggtgaaaaaccttataaatgtccggagtgcggcaagtcatttagctcacgccggacttgcagggcacatcagcgcactcacactggagagaaaccatacaaatgtcccgagtgtggcaagtccttcagccaaaaaagtagcctgatagcacaccaaagaacccacacaggcgagaaaccgtataagtgccctgaatgtggcaagagtttttcccaacgggccaatctgcgggcgcaccaacgcacacatacgggtgagaagccctacaagtgccccgagtgcggtaagagttttagcaggagtgatgagctggtgaggcaccaaaggacgcataccggtgagaagccttacaagtgcccagagtgtgggaaatcattcagtaccacaggggctcttacggagcatcaaagaacgcatacaggcaagaaaacttctcccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtacccctacgatgtaccggattacgcttgataa

SEQ ID NO: 72 - DNA, 1494 bp, synthetic construct
  source 1..1494: mol_type = other DNA
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctggggtcaggagagggcagaggaagtcttctaacatgcggtgacgtggaggagaatcccggccctctcgaacccggagaaaaaccttacaagtgtcctgagtgtggaaagagtttctctagccccgctgatctgacgcgccaccaacgcactcacacaggggagaaaccatacaagtgcccagaatgtggaaaaagcttctcatcacctgccgacctcacaagacatcaacgaactcacactggagaaaaaccatataagtgtcccgaatgtggtaagagcttttctacacatttggatttgatacggcatcaaaggacgcatacaggggagaaaccgtacaaatgcccagagtgtggcaaatcctttagtcaaaacagtacattgactgagcatcagcggactcatacgggagaaaagccctacaagtgtccggagtgtggaaagtctttttccacccatctggacctcattcgacaccaacggacacataccggcgaaaagccttataagtgccccgaatgcggaaaaagcttctctcaatctagtaatcttgtgcgccatcagaggacacacacaggtaaaaaaacgtctcccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtacccctacgatgtaccggattacgcttgataa

SEQ ID NO: 73 - DNA, 1494 bp, synthetic construct
  source 1..1494: mol_type = other DNA
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctggggtcaggagagggcagaggaagtcttctaacatgcggtgacgtggaggagaatcccggccctctggagcctggggaaaaaccctataagtgtccagaatgcggaaagagttttagcgatccaggccatttggtgcgccaccagcgcactcacacgggcgagaaaccctataaatgcccggaatgtggaaaatcattcagcagatccgatgagcttgtccggcaccagaggacgcacaccggtgagaagccctacaaatgcccggaatgcggaaagtcattcagccgcgcggacaatcttactgaacatcagaggactcacacgggtgagaaaccgtacaagtgccccgaatgcgggaaaagtttctctcagtctggtgacctgcggcggcaccagcgaacacatacgggggagaaaccatataaatgcccagagtgcggcaaaagctttagccgacgggacgagctgaatgtacatcagcggacccacacaggtgaaaagccatataagtgccctgagtgcggcaagtccttttctcagttggcacacttgcgggcacatcaacggacacacacgggtaaaaaaacaagccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtacccctacgatgtaccggattacgcttgataa

SEQ ID NO: 74 - DNA, 1494 bp, synthetic construct
  source 1..1494: mol_type = other DNA
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctggggtcaggagagggcagaggaagtcttctaacatgcggtgacgtggaggagaatcccggccctctggagccaggagagaagccttataagtgcccagaatgtggaaaatctttttctgacaagaaagatctgacacgccaccagagaacacacacgggagaaaagccgtacaaatgtcccgaatgtggcaaatcattctcacaatcctcttcccttgtaagacaccaaaggacacacactggcgaaaagccttataagtgccccgagtgtgggaaaagcttcagtcaacgagcccacctggagcggcaccagagaacacatacaggggaaaaaccgtataagtgcccggagtgtggtaagagtttttcccggaaagacaatctcaagaatcatcagcgaacgcatactggagaaaagccttacaagtgtccagagtgtggcaaatctttcagccagagggccaacctcagagcacatcaacgcacacacacgggggagaaaccgtataaatgcccagaatgcggtaaaagcttttctcagaagagtagtcttattgcgcatcaacggacacacacgggaaagaaaaccagtcccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtacccctacgatgtaccggattacgcttgataa

SEQ ID NO: 75 - DNA, 1536 bp, synthetic construct
  source 1..1536: mol_type = other DNA
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctggggtcaggagagggcagaggaagtcttctaacatgcggtgacgtggaggagaatcccggccctatatccgaatttggtagctcatctagtgtcgcgcaagcggctttggagccgggtgagaaaccctacgcatgtccggaatgcggcaaatccttttcccggtctgatcacctggcggagcaccaaaggactcacacgggtgaaaaaccgtataaatgtccagagtgtggtaaaagctttagcgataaaaaagatcttacaaggcaccagcggacgcatacgggcgagaaaccttacaaatgccccgagtgcggcaaatcattttcccaaagagctaacctgcgagcacaccaaagaactcacactggagagaagccatatgcctgtcccgaatgcgggaagagtttctctcagttggcccaccttagagctcaccagcgaacgcatactggcgaaaagccatacaagtgtccggaatgtggtaaaagtttcagccgcgaagatctgaatcacactcaccagaggactcataccggcgagaaaccgtacaagtgtccggagtgcggtaagtccttttcaagaagagacgcgttgaacgtccaccagcgcacccacactggtaagaagacgagtcccaagaagaagaagaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtacccctacgatgtaccggattacgcttgataa

SEQ ID NO: 76 - DNA, 1494 bp, synthetic construct
  source 1..1494: mol_type = other DNA
atggcctcctccgaggacgtcatcaaggagttcatgcgcttcaaggtgcgcatggagggctccgtgaacggccacgagttcgagatcgaaggagaaggtgaaggaagaccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctggggtcaggagagggcagaggaagtcttctaacatgcggtgacgtggaggagaatcccggccctctcgagccaggcgagaaaccttataaatgtcccgaatgtggcaaatccttcagtgagagaagtcaccttcgggaacaccagagaacccacacaggggaaaaaccttataagtgccctgaatgcggcaaatccttctccagtaaaaaacacctcgcggaacatcaaagaacccacacaggtgagaagccatataagtgcccggaatgtggtaagtccttttccagtccggctgatttgactaggcatcagcgcacgcatacgggtgagaagccgtataaatgcccggaatgcggcaagtctttcagtgatccgggccacctggtccgacatcaacggactcatactggagaaaaaccatacaagtgcccggaatgcggaaagagctttagtacatccggaagtctcgtcagacaccaacgcactcatacgggcgaaaagccttacaaatgccccgagtgtggtaagtccttcagcacgagtgggagtcttgtgcggcaccagcggacacacacaggaaagaaaacgagtcccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtacccctacgatgtaccggattacgcttgataa

SEQ ID NO: 77 - DNA, 1494 bp, synthetic construct
  source 1..1494: mol_type = other DNA
atggcctcctccgaggacgtcatcaaggagttcatgcgcttcaaggtgcgcatggagggctccgtgaacggccacgagttcgagatcgaaggagaaggtgaagggcgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctggggtcaggagagggcagaggaagtcttctaacatgcggtgacgtggaggagaatcccggccctttggagccgggtgagaagccttataaatgtcctgagtgcgggaaaagtttcagccatactggacaccttctcgaacaccagaggacgcacaccggggagaagccatataaatgccccgagtgtggtaagagttttagttcacgcggcaacctcaaaagccatcaaaggactcatacaggagagaagccatacaaatgtcctgaatgtggcaaaagcttctcttcaaagaaggcgcttactgagcaccagcgcactcacactggcgaaaagccgtacaaatgtccggaatgcgggaagagttttagtgattgtcgggacttggcaaggcaccagcgcacgcataccggggaaaaaccatacaaatgcccggagtgcgggaagtccttttcacgatctgatcatctgacaaaccaccagcggacgcacacgggggagaagccttacaaatgccccgaatgcggcaaatccttttctgcaccgaaggctcttgggtggcatcagcgaacacataccggaaaaaagacaagtcccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtacccctacgatgtaccggattacgcttgataa

SEQ ID NO: 78 - DNA, 1494 bp, synthetic construct
  source 1..1494: mol_type = other DNA
atggcctcctccgaggacgtcatcaaggagttcatgcgcttcaaggtgcgcatggagggctccgtgaacggccacgagttcgagatcgaaggagaaggagaaggtagaccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctggggtcaggagagggcagaggaagtcttctaacatgcggtgacgtggaggagaatcccggccctcttgaacccggggagaaaccgtataagtgtccagagtgcggcaagagcttttcccgcacagacacacttcgggaccatcagcggacacatacgggagagaagccttataaatgccccgaatgcggaaaatctttctccaggagtgataaactggttcggcaccaacgcacgcatacaggtgaaaagccttacaaatgccccgagtgtggtaagtccttctcagatccagggcacctggtgaggcaccaacggactcacaccggcgagaaaccatacaagtgccccgagtgcggcaaaagcttcagcaggcgcgatgaactcaatgttcaccagaggacacacactggagagaaaccgtacaagtgtccggaatgcggcaagagtttttctcggtctgatcatttgaccaaccaccaacgaacgcacactggcgaaaagccatataaatgtcccgagtgcggaaagtctttctctcggaaggacgctctgagaggtcatcaacggactcacacaggcaaaaagactagtcccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtacccctacgatgtaccggattacgcttgataa

SEQ ID NO: 79 - DNA, 1494 bp, synthetic construct
  source 1..1494: mol_type = other DNA
atggcctcctccgaggacgtcatcaaggagttcatgcgcttcaaggtgcgcatggagggctccgtgaacggccacgagttcgagatcgaaggagaaggagaaggaagaccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctggggtcaggagagggcagaggaagtcttctaacatgcggtgacgtggaggagaatcccggccctctcgagccgggtgaaaaaccgtacaagtgtcccgagtgtggcaagtctttctctcagtcaggtcatttgacggaacatcagagaacacatacaggagaaaaaccctataagtgtccggaatgtggaaaatccttttcacataggacgacgttgaccaatcatcaacggactcacaccggcgagaaaccctacaagtgtccagaatgtgggaagtctttttctcaacgggcgcacctggaaagacaccagcggacgcacactggcgaaaaaccctataaatgtcctgaatgcgggaagagtttctcagctaggggaaacctccggactcaccagagaacccacacgggagaaaagccatataagtgtcctgagtgcggaaaatcattctccgaccctggacaccttgttcgccatcagcgcacgcacacaggagagaaaccgtataaatgccccgaatgcggcaaatctttcagccgagcggacaatctcactgagcaccaacgcacacacactggtaagaaaacgagccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtacccctacgatgtaccggattacgcttgataa

SEQ ID NO: 80 - DNA, 18 bp, Homo sapiens
  source 1..18: mol_type = genomic DNA
agaatggcacaggtgggc

SEQ ID NO: 81 - DNA, 18 bp, Homo sapiens
  source 1..18: mol_type = genomic DNA
cagggctatggaagtcga

SEQ ID NO: 82 - DNA, 441 bp, Homo sapiens
  source 1..441: mol_type = genomic DNA
gcgaagtggggaggtacaaaaaaatttccagtccttgaatggtgtgaagtaaaagtgccttcaaagaatcccaccagaatggcacaggtgggcataatgggtctgtctcatcgtcaaaggacccaaggagtctaaaggaaactctaactacaacacccaaatgccacaaaaccttagttattaatacaaactatcatccctgcctatctgtcaccatctcatcttaaaaaacttgtgaaaatacgtaatcctcaggagacttcaattaggtataaataccagcagccagaggaggtgcagcacattgttctgatcatctgaagatcagctattagaagagaaagatcagttaagtcctttggacctgatcagcttgatacaagaactactgatttcaacttctttggcttaattctctcggaaacgatgaaatatacaagt

SEQ ID NO: 83 - protein, 5 aa, Homo sapiens
MKYTS

SEQ ID NO: 84 - DNA, 1157 bp, Homo sapiens
  source 1..1157: mol_type = genomic DNA
gctctgaggaatgggttacaggagacctctggggagatgtgaccacagcaatgggtaggagaatgtccagggctatggaagtcgagtatggggacccccccttaacgaagacagggccatgtagagggccccagggagtgaaagagcctccaggacctccaggtatggaatacaggggacgtttaagaagatatggccacacactggggccctgagaagtgagagcttcatgaaaaaaatcagggaccccagagttccttggaagccaagactgaaaccagcattatgagtctccgggtcagaatgaaagaagaaggcctgccccagtggggtctgtgaattcccgggggtgatttcactccccggggctgtcccaggcttgtccctgctacccccacccagcctttcctgaggcctcaagcctgccaccaagcccccagctccttctccccgcagggacccaaacacaggcctcaggactcaacacagcttttccctccaaccccgttttctctccctcaaggactcagctttctgaagcccctcccagttctagttctatctttttcctgcatcctgtctggaagttagaaggaaacagaccacagacctggtccccaaaagaaatggaggcaataggttttgaggggcatggggacggggttcagcctccagggtcctacacacaaatcagtcagtggcccagaagacccccctcggaatcggagcagggaggatggggagtgtgaggggtatccttgatgcttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagccagcagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagc

SEQ ID NO: 85 - protein, 5 aa, Homo sapiens
MSTES